(19) United States
(12) Patent Application Publication
(10) Pub. No.: US 2024/0018129 A1
(43) Pub. Date: Jan. 18, 2024

(54) COMPOUNDS AS PU. 1 INHIBITORS

(71) Applicant: PEKING UNIVERSITY, Beijing (CN)

(72) Inventors: Xiaoguang LEI, Beijing (CN); Hong WU, Beijing (CN); Xin WANG, Beijing (CN); Ningning YAO, Beijing (CN); Fusheng GUO, Beijing (CN)

(73) Assignee: S-Infinity Pharmaceuticals, Huzhou (CN)

(21) Appl. No.: 18/253,726

(22) PCT Filed: Nov. 18, 2021
(86) PCT No.: PCT/CN2021/131434

§ 371 Date: May 19, 2023

(30) Foreign Application Priority Data
Nov. 20, 2020 (WO) PCT/CN2020/130512

(51) Int. Cl. C07D 403/14; C07D 519/00; A61P 35/02; A61P 17/00; A61P 11/00; A61P 1/16

(52) U.S. Cl. CPC C07D 403/14; C07D 519/00; A61P 35/02; A61P 17/00; A61P 11/00; A61P 1/16

(57) ABSTRACT

Disclosed herein are compounds of formula (I) that are PU.1 inhibitors. Also provided herein are methods or preparing these compounds.

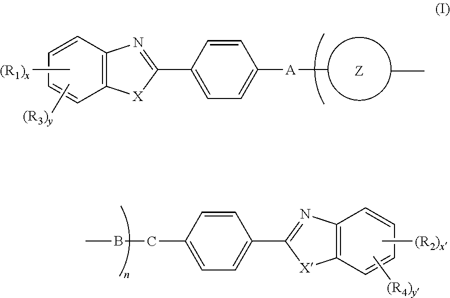

### CROSS-REFERENCE TO RELATED APPLICATION

This application claims priority benefit of PCT International Application No. PCT/CN2020/130512, filed Nov. 20, 2020, the disclosure of which is hereby incorporated herein by reference in its entirety.

### FIELD

This disclosure relates to novel inhibitors of transcription factor PU.1, their chemical synthesis, and their applications for the treatment of disorders like leukemia and fibrosis.

### BACKGROUND

T cell acute lymphoblastic leukemia (T-ALL) is a type of hematopoietic cancer resulting from abnormal expansion of T cell progenitors. It accounts for 15% in pediatric patients and 25% in adults. T-ALL is a heterogeneous disease in both biological process and genetic level. Belver, L. & Ferrando, A. Nat. Rev. Cancer 16, 494-507, doi:10.1038/nrc.2016.63 (2016).Despite the heterogeneous characteristic of T-ALL, the main genetic lesions include chromosomal translocations that affect some oncogene expression and mutations or deletions of some genes that are associated with signaling pathways or the cell cycle. Teachey, E. A. R. a. D. T. Hematology, 8 (2016). Aberrant activation of NOTCH1 accounts for about 60% in T-ALL cases, Tosello, V. & Ferrando, A. A. Therapeutic advances in hematology 4, 199-210, doi:10.1177/2040620712471368 (2013), and the deletion or mutation of a well-known tumor suppressor, PTEN, is reported to account for about 20% in T-ALL patients. Guan, W., Jing, Y. & Yu, L. Zhongguo shi yan xue ye xue za zhi 25, 587-591, doi:10.7534/j.issn.1009-2137.2017.02.050 (2017). Intensive and high-dose chemotherapy could improve the outcomes of the T-ALL patients, but some patients who relapse and then re-treated with chemotherapy would still die due to the disease. Pui, C. H., Sailan, S., Relling, M. V., Masera, G. & Evans, W. E. Leukemia 15, 707-715, doi:10.1038/sj.leu.2402111 (2001); Nguyen, K. et al. Leukemia 22, 2142-2150, doi:10.1038/leu.2008.251 (2008); Reismueller, B. et al. Journal of Pediatric Hematology Oncology 35, E200-E204, doi:10.1097/MPH.0b013e318290c3d6 (2013).The most important factor of drug resistance is the existence of leukemia-initiating cells (LICs). LICs have the ability of self-renew and differentiating into leukemia blast cells. Previous study has reported that leukemia blast cells but not LICs could be eliminated by targeting the activated pathways. LIC is a tricky population in T-ALL targeting therapy.

To investigate the development and mechanism of leukemia, a Pten-null T-ALL model was established. In the model, Pten was 40% deleted in mouse fetal liver hematopoietic stem cells, followed by activation of PI3K-AKT pathway, overexpression of c-Myc oncogene, and disruption of the hematopoietic system. In about two month from born, the mice would develop aggressive T-ALL. Guo, W. et al., Nature 453, 529-533, doi:10.1038/nature06933 (2008). Using c-kit, a stem-state-like marker, we could separate T ALL cells into blast cells and LICs. Following work in our lab has identified that TIM-3 is an important surface marker that is highly expressed in membrane of LICs, but not in blast and normal cells. PU.1, an ETS-family transcription factor, can bind with TIM-3 promoter and regulate TIM-3 expression, as well as sustain the “sternness” of LICs. In the LICs, the expression levels of TIM-3 and PU.1 are highly correlated. A series of LICs signature genes are potential PU.1 targets. Zhu, H. et al., eLife 7, doi:10.7554/eLife.38314 (2018).

PU.1 is a transcription factor belonging to ETS family and plays important roles in hematopoiesis. Its expression level is different in various hematopoietic progenitors and their progeny. In long-term HSCs (LT-HSC), the expression level of PU.1 is low, yet when differentiating into progenitors, for example, CMP and CLP, PU.1 are highly expressed. PU.1's expression is also different in various mature lineages, with high expression in macrophages than B cells and lower levels in T cells, erythroid cells and megakaryocytes. In GMP population, PU.1's expression in their progeny neutrophils and monocytes is highly needed. Several mouse models have demonstrated the role of PU.1 in myelopoiesis. Deletion of PU.1 would lead to lack of CMPs, absence of mature macrophages. Besides, PU.1 is important for committed myeloid cells, as it can regulate the expression of several myeloid-specific genes, including GM-CSFRa, G-CSFR, M-CSFR and IL-7R. In addition to being a master regulator of myeloid lineage, PU.1 also plays important roles in regulating lymphoid lineage differentiation, and the process that gives rise to B and T lineage and lineage choice. Study that used mice with GFP reporter has identified that PU.1 expression level increases as B cell matures, but is silenced in mature T cells. PU.1-null CLPs could generate B cells. Similar to B cells, PU.1 is required in T-progenitor stage but decreases in mature T cells. If PU.1 is overexpressed in mature T cells, the cells may show stem-cell like state and growth arrest, as well as maturation block. Mak, K. S. et al., International Journal of Cell Biology 2011, 808524, doi:10.1155/2011/808524 (2011). Recently, it was shown that PU.1 can control fibroblast polarization and tissue fibrosis, and PU.1 inhibition may represent a promising therapeutic approach to treat a wide range of fibrotic diseases. Wohlfahrt, T. et al., Nature 566, 344-349, doi:10.1038/s41586-019-0896-x (2019). Moreover, PU.1 inhibitors can decrease cell growth and the clonogenic capacity of acute myeloid leukemia (AML) cells, leading to increased apoptosis of AML cells, and PU.1 inhibition has potential as a therapeutic strategy for the treatment of AML. Antony-Debre, I. et al., J Clin Invest 127, 4297-4313, doi:10.1172/JC192504 (2017).

Fibrosis is a restorative or reactive process that is characterized by the formation and deposition of excessive fibrous connective tissue and extracellular matrix, leading to progressive structural remodeling and further failure of almost all tissues and organs, such as, lung, skin, liver, kidney, heart and others. Rockey, D. C. et al., N Engl J Med 373, 96, doi:10.1056/NEJMc1504848 (2015). Consequently, fibrosis is a serious factor for inducing morbidity and mortality and is estimated to cause more than 45% of all death in the United States. Wynn, T. A., Nat Rev Immunol 4, 583-594, doi:10.1038/nri1412 (2004). Under stimulants, such as, a wound healing or inflammatory response, fibroblasts differentiate into a matrix-producing phenotype and promote accumulation of extracellular matrix, which is an initiatory switch of fibrosis disease. Palumbo-Zerr, K. et al., Nat Med 21, 150-158, doi:10.1038/nm.3777 (2015); Ramming, A. et al., Pharmacol Res 100, 93-100, doi:10.1016/j.phrs.2015.06.012 (2015); Chakraborty, D. et al., Nat Commun 8, 1130, doi:10.1038/s41467-017-01236-6 (2017). Then the accompanying inflammatory response will lead to the activation of immune cells (mainly tissue macrophages) and participate in the fibrosis-mediated homeostasis balance regulation. At present, there are few approaches and limited efficacy to treat organ fibrosis.

Non-alcoholic fatty liver disease (NAFLD) is caused by abnormal and large fat accumulation (steatosis) in liver without excessive alcohol, then processes to steatohepatitis (non-alcoholic steatohepatitis, NAASH) and fibrosis with inflammatory response and collagen deposition, which may evolve into cirrhosis and carcinoma. Adams, L. A. et al., J Hepatol 62, 1002-1004, doi:10.1016/j.jhep.2015.02.005 (2015); Ratziu, V., Lancet 385, 922-924, doi:10.1016/50140-6736(14)62010-9 (2015). Not only more than a third of people in developed countries suffer from hepatic steatosis, and tend to be younger, but NASH-mediated liver failure is a leading issue for liver transplantation. Cohen, J. C. et al., Science 332, 1519-1523, doi:10.1126/science.1204265 (2011); Stine, J. G. et al., Liver Transp 21, 1016-1021, doi:10.1002/lt.24134 (2015). Unfortunately, the pharmacological intervention for NASH is poor, only a PPARα/γ agonist Saroglitazar has been approved by drug-controller general of India. Therefore, there is an urgent need to understand how fibrosis occurs and develops, to discover novel targets for drug discovery and identify potential therapeutic approaches to treat NASH and organ fibrosis.

Previous study has shown that ETS-family transcription factor PU.1 is the master regulator of LIC signature genes, and PU.1 is essential for LICs “sternness” and T-ALL development. Zhu, H. et al., eLife 7, doi:10.7554/eLife.38314 (2018). Besides, it was reported that PU.1 is highly expressed in fibrotic fibroblasts, but silenced in matrix-degrading fibroblasts, and PU.1 inhibitor DB1976 treatment can alleviated skin, liver and lung fibrosis. Wohlfahrt, T. et al., Nature 566, 344-349, doi:10.1038/s41586-019-0896-x (2019). We and cooperators also identified that PU.1 inhibition, mediated by DB1976 or shRNA application, shows beneficial effects on NASH progress, including reduced liver steatosis, inflammation, fibrosis and improved glucose homeostasis in vivo. Liu, Q. et al. J Hepatol 73, 361-370, doi:10.1016/j.jhep.2020.02.025 (2020). These works indicated that PU.1 is a potential and effective target for drug discovery and development for leukemia, liver disorders and multiple organ fibrosis. But the existing PU.1 inhibitor, for example, DB1976, has limited potency, and owns inhibitory activity on other ETS family numbers, which is a potential risk for further drug development.

There is a need for improved methods for treating hematologic T-ALL, as well as other conditions associated with PU.1 dysfunction, for example, NASH and organ fibrosis, with novel potent and selective PU.1 inhibitors owning to scientific significance and potential medicinal value. The present disclosure addresses this need.

### SUMMARY

The present disclosure provides compounds that can block the interaction of ETS family transcription factor PU.1 with target DNA, downregulate the expression of TIM-3, kill leukemia cells efficiently and alleviate organ fibrosis. These compounds may have wide applications for treating disorders such as leukemia and fibrosis.

In one aspect, provided is a compound of formula (I), or a stereoisomer or a pharmaceutically acceptable salt thereof,

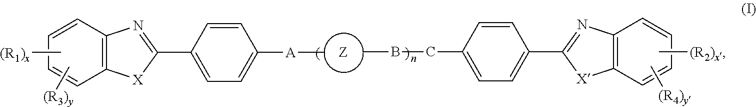

wherein X, X′, x, x′, y, y′, R₁, R₂, R₃, R₄, A, Z, B, C, and n are as disclosed herein.

In another aspect, provided is a method of preparing the compound of formula (I), or a stereoisomer or a pharmaceutically acceptable salt thereof, comprising converting a compound of formula (II)

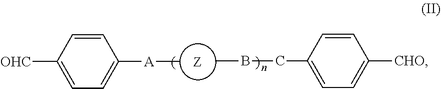

or a stereoisomer or a pharmaceutically acceptable salt thereof, to the compound of Formula (I), or a stereoisomer or a pharmaceutically acceptable salt thereof, wherein X, X′, x, x′, y, y′, R₁, R₂, R₃, R₄, A, Z, B, C, and n are as disclosed herein.

In another aspect, provided is a method of treating a PU.1-mediated disease in an individual in need thereof, comprising administering an effective amount of a compound as described herein, or a stereoisomer or a pharmaceutically acceptable salt thereof, to the individual. In some embodiments, provided is a compound as described herein, or a stereoisomer or a pharmaceutically acceptable salt thereof, for use in treating a PU.1-mediated disease. In some embodiments, provided is use of a compound as described herein, or a stereoisomer, or a pharmaceutically acceptable salt thereof, in the manufacture of a medicament for the treatment of a PU.1-mediated disease. In some embodiments, the PU.1-mediated disease is leukemia or fibrosis. In some embodiments, the PU.1-mediated disease or disorder is acute lymphoblastic leukemia (ALL), acute myeloid leukemia (AML), chronic lymphoblastic leukemia (CLL), chronic myeloid leukemia (CML), skin fibrosis, pulmonary fibrosis, renal fibrosis, liver fibrosis, or cardiac fibrosis. In some embodiments, the PU.1-mediated disease is NASH.

In another aspect, provided is a composition, such as a pharmaceutical composition, comprising a compound as described herein, or a stereoisomer or a pharmaceutically acceptable salt thereof, and a pharmaceutically acceptable carrier or excipient. Also provided is a kit comprising a compound as described herein, or a stereoisomer or a pharmaceutically acceptable salt thereof.

### BRIEF DESCRIPTION OF THE DRAWINGS

FIG. 1 shows a rational design of novel small molecule PU.1 inhibitors abrogating DNA binding by PU.1 and biological evaluation of potency of PU.1 inhibitors. (a) Difference between the instantly disclosed PU.1 inhibitors, such as compound I-1, and DB2115 (replacement of flexible linker with rigid or AT-selective linkers.) (b) q-PCR analysis of compounds' activity in inhibiting LICs' signature gene TIM-3 in Blast-PU.1 cells (24-hour treatment).

FIG. 2 shows the effects of compound I-1 and rapamycin combination treatment on Leukemia burden in Pten-null T-ALL mice. (a) Blast and Tim-3 high LIC proportion in mouse bone marrow after compound I-1 and Rapamycin or combination treatment. (b) Hematoxylin-eosin (H&E) staining of compound I-1 and Rapamycin or combination treatment mouse organs. Scale bar, 300 μm. (c) Compound I-1 and Rapamycin or combination treatment mouse spleen immunohistochemistry (1HC) analysis of murine B220 (CD45R). Scale bar, 100 μm. (d) Survival curve of compound I-1 (left) or DB1976 (right) and Rapamycin as well as combination treatment of Pten-null T-ALL mice.

FIG. 3 shows the preventive and therapeutic effects of compound I-1 on skin fibrosis disease (compound I-1, 5mpk; DB1976, 5mpk; vehicle, saline.) (a-e) compound I-1 prevents bleomycin-induced skin fibrosis (n=6). (f-j) compound I-1 alleviates and reverses bleomycin-induced skin fibrosis (n=6). (a and f) Experimental design of preventive and therapeutic models of bleomycin-induced skin fibrosis. (b and g) Pathological sections and staining of skin from different groups. Top, H&E staining; middle, Sirius red staining; bottom, Masson staining. Scale, 500 μm. (c and h) Quantized epidermal skin thickness. (d-e and i-j) Relative mRNA level of Col1a1 and Col1a2 level, normalized by GAPDH. Data are shown as the mean±s.e.m. of respective n biologically independent samples. P values were determined by one-way ANOVA with Tukey's multiple comparison post hoc test. *P<0.05, **.P<0.01 and ***P<0.001 versus bleomycin/vehicle group.

FIG. 4 shows the preventive and therapeutic effects of compound I-1 on pulmonary fibrosis disease (compound I-1, 5mpk; DB1976, 5mpk; vehicle, saline.) (a-g) compound I-1 prevents bleomycin-induced pulmonary fibrosis (n=5). (h-n) compound I-1 alleviates and reverses bleomycin-induced pulmonary fibrosis (n=5). (a and h) Experimental design of preventive and therapeutic models of bleomycin-induced pulmonary fibrosis. (b and i) The lung photos after the mentioned treatment. (c and j) H&E staining of lung from different groups. Left, lower magnification, scale, 500 μm; right, higher magnification, 100 μm. (d and k) Ashcroft scores. (e and 1) Sirius red staining of lung from different groups. Left, lower magnification, scale, 500 μm; right, higher magnification, 100 μm. (f-g and m-n) Relative mRNA level of Col1a1 and Col1a2 level, normalized by GAPDH. Data are shown as the mean±s.e.m. of respective n biologically independent samples. P values were determined by one-way ANOVA with Tukey's multiple comparison post hoc test. *P<0.05, **P<0.01 and ***P<0.001 versus bleomycin/vehicle group.

FIG. 5 shows the effects of compound I-1 on liver lipid accumulation and treatment of NASH. (a) Experimental design of NASA diet—induced model and compounds treatment arrangement, n=8. (b-c) Body weight and liver/body weight radio in different groups at the final time point. (d) H&E staining of liver tissues after tissues collection. Scale, 250 μm (e) Oil red 0 staining of liver tissues after tissues collection. Top, lower magnification, scale, 250 μm; bottom, higher magnification, scale, 50 μm. (1) NAFLD activity score following standard. (g-i) ALT, LDL-C and Total cholesterol level in serum from different groups. (j-k) Inflammation-related gene, IL-6 and IL-1β mRNA level. (1-m) fibrosis-related genes, Col1a1, Col1a2 mRNA level. Normalized by GAPDH. Data are shown as the mean±s.e.m. of respective n biologically independent samples. P values were determined by one-way ANOVA with Tukey's multiple comparison post hoc test. *P<0.05, **P<0.01 and ***P<0.001 versus vehicle/NASH diet group.

FIG. 6 shows the effects of compound I-1 on HFD/CCL4-induced NASH and liver fibrosis in mice. (a) Experimental design of HFD/CCL4-induced NASH and liver fibrosis model and compounds treatment arrangement, (n=6-8). (b) Body weight of different groups in the final time point. (c-d) Weights of white adipose tissue (inguinal white fat, iWAT; gonadal white fat, gWAT) radio in different groups. (e-f) Fasting serum levels of triglyceride (TG) and total-cholesterol (TC) of mediated groups. (g) H&E and Sirius red staining of liver tissues after tissues collection. For H&E staining, Top, lower magnification, scale, 250 μm; bottom, higher magnification, scale, 50 μm. For Sirius red staining, Scale, 500 μm. (h-i) Liver steatosis and inflammation scores based on H&E staining. (j-k) Inflammation-related gene, IL-6 and IL-1β mRNA level in livers. Normalized by GAPDH. (1) Sirius red—positive area quantitative data based on Sirius red staining. (m-n) Fibrosis-related genes, Col1a1, Col1a2 mRNA level in livers. Normalized by GAPDH. (o) Fasting serum ALT level of different groups. Data are shown as the mean±s.e.m. of respective n biologically independent samples. P values were determined by one-way ANOVA with Tukey's multiple comparison post hoc test. *P<0.05, **P<0.01 and ***P<0.001 versus Vehicle (saline)/HFD+CCL4 group.

FIG. 7 shows the effects of compound I-1 on CCL-induced liver fibrosis. (a) Experimental design of CCL4-induced liver fibrosis model and compounds treatment arrangement, (n=6). (b-c) Sirius red staining of liver tissues after tissues collection and Sirius red—positive area quantitative data. Scale, 500 μm. (d-e) Fibrosis-related genes, Col1a1, Col1a2 mRNA level. Normalized by GAPDH. (f) H&E staining of liver tissues after tissues collection. Top, lower magnification, scale, 250 μm; bottom, higher magnification, scale, 50 μm. (g-h) Inflammation-related gene, IL-6 and IL-1β mRNA level. Normalized by GAPDH. (i) AST level in serum from different groups. Data are shown as the mean±s.e.m. of respective n biologically independent samples. P values were determined by one-way ANOVA with Tukey's multiple comparison post hoc test. *P<0.05, **P<0.01 and ***P<0.001 versus CCL4/vehicle group.

### DETAILED DESCRIPTION

The following description sets forth exemplary embodiments of the present disclosure. It should be recognized, however, that such description is not intended as a limitation on the scope of the present disclosure but is instead provided as a description of exemplary embodiments.

### Definitions

As used in the present specification, the following words, phrases and symbols are generally intended to have the meanings as set forth below, except to the extent that the context in which they are used indicates otherwise.

The term “about” refers to a variation of ±1%, ±3%, ±5%, or ±10% of the value specified. For example, “about 50” can in some embodiments includes a range of from 45 to 55. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. Reference to “about” a value or parameter herein includes (and describes) embodiments that are directed to that value or parameter per se. For example, description referring to “about X” includes description of “X”.

The singular forms “a” and “the” include plural references unless the context clearly dictates otherwise. Thus, e.g., reference to “the compound” includes a plurality of such compounds and includes reference to one or more compounds and equivalents thereof known to those skilled in the art.

“Alkyl” refers to an unbranched or branched saturated hydrocarbon chain. As used herein, alkyl has 1 to 10 carbon atoms (i.e., C₁₋₁₀ alkyl or C₁-C₁₀ alkyl), 1 to 8 carbon atoms (i.e., C₁₋₈ alkyl or C₁-C₈ alkyl), 1 to 6 carbon atoms (i.e., C₁₋₆ alkyl or C₁-C₆ alkyl), or 1 to 4 carbon atoms (i.e., C₁₋₄ alkyl or C₁-C₄ alkyl). Examples of alkyl groups include, without limitation, methyl, ethyl, propyl, isopropyl, n-butyl, sec-butyl, isobutyl, tert-butyl, pentyl, 2-pentyl, isopentyl, neopentyl, hexyl, 2-hexyl, 3-hexyl and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂). It is understood that the term “alkyl” also contemplates a divalent moiety.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Alkoxyl” refers to the group “—O-alkyl”. Examples of alkoxyl groups include, without limitation, methoxy, ethoxy, n-propoxy, isopropoxy, n-butoxy, tert-butoxy, sec-butoxy, n-pentoxy, n-hexoxy and 1,2-dimethylbutoxy.

“Aryl” refers to an aromatic carbocyclic group having a single ring (e.g., monocyclic) or multiple rings (e.g., bicyclic or tricyclic) including fused systems. As used herein, aryl has 6 to 20 ring carbon atoms (i.e., C₆₋₂₀ aryl or C₆-C₂₀ aryl), 6 to 12 carbon ring atoms (i.e., C₆₋₁₂ aryl or C₆-C₁₂ aryl), or 6 to 10 carbon ring atoms (i.e., C₆₋₁₀ aryl or C₆-C₁₀ aryl). Examples of aryl groups include, without limitation, phenyl, naphthyl, fluorenyl and anthryl. Aryl, however, does not encompass or overlap in any way with heteroaryl defined below. If one or more aryl groups are fused with a heteroaryl, the resulting ring system is heteroaryl. If one or more aryl groups are fused with a heterocyclyl, the resulting ring system is heterocyclyl. It is understood that the term “aryl” also contemplates a divalent moiety.

“Cycloalkyl” refers to a saturated or partially unsaturated cyclic alkyl group having a single ring or multiple rings including fused, bridged and spiro ring systems. The term “cycloalkyl” includes cycloalkenyl groups (i.e., the cyclic group having at least one double bond) and carbocyclic fused ring systems having at least one sp³ carbon atom (i.e., at least one non-aromatic ring). As used herein, cycloalkyl has from 3 to 20 ring carbon atoms (i.e., C₃₋₂₀ cycloalkyl or C₃-C₂₀ cycloalkyl), 3 to 12 ring carbon atoms (i.e., C₃₋₁₂ cycloalkyl or C₃-C₁₂ cycloalkyl), 3 to 10 ring carbon atoms (i.e., C₃₋₁₀ cycloalkyl or C₃-C₁₀ cycloalkyl), 3 to 8 ring carbon atoms (i.e., C₃₋₈ cycloalkyl or C₃-C₈ cycloalkyl), or 3 to 6 ring carbon atoms (i.e., C₃₋₆ cycloalkyl or or C₃-C₆ cycloalkyl). Monocyclic groups include, without limitation, cyclopropyl, cyclobutyl, cyclopentyl, cyclohexyl, cycloheptyl and cyclooctyl. Further, the term cycloalkyl is intended to encompass any non-aromatic ring which may be fused to an aryl ring, regardless of the attachment to the remainder of the molecule. Still further, cycloalkyl also includes “spirocycloalkyl” when there are two positions for substitution on the same carbon atom. It is understood that the term “cycloalkyl” also contemplates a divalent moiety.

“Heteroaryl” refers to an aromatic group having a single ring, multiple rings or multiple fused rings, with one or more ring heteroatoms independently selected from nitrogen, oxygen and sulfur. As used herein, heteroaryl includes 1 to 20 ring carbon atoms (i.e., C₁₋₂₀ heteroaryl), 3 to 12 ring carbon atoms (i.e., C₃₋₁₂ heteroaryl), or 3 to 8 carbon ring atoms (i.e., C₃₋₈ heteroaryl) and 1 to 5 ring heteroatoms, 1 to 4 ring heteroatoms, 1 to 3 ring heteroatoms, 1 to 2 ring heteroatoms, or 1 ring heteroatom independently selected from nitrogen, oxygen and sulfur. In certain instances, heteroaryl includes 5-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. Any aromatic ring, having a single or multiple fused rings, containing at least one heteroatom, is considered a heteroaryl regardless of the attachment to the remainder of the molecule (i.e., through any one of the fused rings). Heteroaryl does not encompass or overlap with aryl as defined above. Examples of heteroaryl groups include, but are not limited to, pyridyl, pyrimidyl, thiophenyl, furanyl, thiazolyl, oxazolyl, isoxazolyl, thiophenyl, pyrrolyl, pyrazolyl, 1,3,4-oxadiazolyl, imidazolyl, isothiazolyl, triazolyl, 1,3,4-thiadiazolyl, tetrazolyl, benzofuranyl, benzothiophenyl, pyrazolopyridinyl, indazolyl, benzothiazolyl, benzooxazolyl, and benzoimidazolyl and the like. It is understood that the term “heteroaryl” also contemplates a divalent moiety.

“Heterocyclyl” refers to a saturated or partially unsaturated cyclic alkyl group, with one or more ring heteroatoms independently selected from nitrogen, oxygen and sulfur. The term “heterocyclyl” includes heterocycloalkenyl groups (i.e., the heterocyclyl group having at least one double bond), bridged-heterocyclyl groups, fused-heterocyclyl groups and spiro-heterocyclyl groups. A heterocyclyl may be a single ring or multiple rings wherein the multiple rings may be fused, bridged or Spiro and may comprise one or more (e.g., 1 to 3) oxo (═O) or N-oxide (N⁺—O⁻) moieties. Any non-aromatic ring containing at least one heteroatom is considered a heterocyclyl, regardless of the attachment (i.e., can be bound through a carbon atom or a heteroatom). Further, the term heterocyclyl is intended to encompass any non-aromatic ring containing at least one heteroatom, which ring may be fused to an aryl or heteroaryl ring, regardless of the attachment to the remainder of the molecule. As used herein, heterocyclyl has 2 to 20 ring carbon atoms (i.e., C₂₋₂₀ or C₂-C₂₀ heterocyclyl), 2 to 12 ring carbon atoms (i.e., C₂₋₁₂ or C₂-C₁₂ heterocyclyl), 2 to 10 ring carbon atoms (i.e., C₂₋₁₀ or C₂-C₁₀ heterocyclyl), 2 to 8 ring carbon atoms (i.e., C₂₋₈ or C₂-C₈ heterocyclyl), 3 to 12 ring carbon atoms (i.e., C₃₋₁₂ or C₃-C₁₂ heterocyclyl), 3 to 8 ring carbon atoms (i.e., C₃₋₈ or C₃-C₈ heterocyclyl), or 3 to 6 ring carbon atoms (i.e., C₃₋₆ or C₃-C₆ heterocyclyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. In certain instances, heterocyclyl includes 3-12 membered ring systems, 5-10 membered ring systems, 5-7 membered ring systems, or 5-6 membered ring systems, each independently having 1 to 4 ring heteroatoms, 1 to 3 ring heteroatoms, 1 to 2 ring heteroatoms, or 1 ring heteroatom independently selected from nitrogen, oxygen and sulfur. The term “heterocyclyl” also includes “spiroheterocyclyl” when there are two positions for substitution on the same carbon atom. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl and the like. It is understood that the term “heterocyclyl” also contemplates a divalent moiety.

“Oxo” refers to ═O.

“Halogen” or “halo” includes fluoro, chloro, bromo and iodo.

The terms “optional” or “optionally” means that the subsequently described event or circumstance may or may not occur.

“Substituted” as used herein means one or more (e.g., 1-8, 1-6, 1-5, 1-4, 1-3, 1-2, 2-5, 2-4, 2-3, 3-5, or 3-4) hydrogen atoms of the group is replaced with the substituents listed for that group, which may be the same or different. “Optionally substituted” means that a group may be unsubstituted or substituted by one or more (e.g., 1-8, 1-6, 1-5, 1-4, 1-3, 1-2, 2-5, 2-4, 2-3, 3-5, or 3-4) substituents listed for that group, wherein the substituents may be the same or different.

Provided are also are stereoisomers, mixture of stereoisomers, tautomers, hydrates, solvates, isotopically enriched analogs and pharmaceutically acceptable salts of the compounds described herein.

The compounds disclosed herein, or their pharmaceutically acceptable salts, may include an asymmetric center and may thus give rise to enantiomers, diastereomers and other stereoisomeric forms that may be defined, in terms of absolute stereochemistry, as (R)- or (S)- or, as (D)- or (L)- for amino acids. The present disclosure is meant to include all such possible isomers, as well as their racemic and optically pure forms. Optically active (+) and (−), (R)- and (S)-, or (D)- and (L)-isomers may be prepared using chiral synthons or chiral reagents or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high-performance liquid chromatography (HPLC). When the compounds described herein contain olefinic double bonds or other centers of geometric asymmetry and unless specified otherwise, it is intended that the compounds include both E- and Z-geometric isomers.

A “stereoisomer” refers to a compound made up of the same atoms bonded by the same bonds but having different three-dimensional structures, which are not interchangeable. The present disclosure contemplates various stereoisomers and mixtures thereof and includes “enantiomers,” which refers to two stereoisomers whose molecules are nonsuperimposable mirror images of one another and “diastereomers,” which refers to stereoisomers that have at least two asymmetric atoms, but which are not mirror-images of each other. Thus, all stereoisomers (for example, geometric isomers, optical isomers and the like) of the present compounds (including those of the salts, solvates and hydrates of the compounds), such as those which may exist due to asymmetric carbons on various substituents, including enantiomeric forms (which may exist even in the absence of asymmetric carbons), rotameric forms, atropisomers and diastereomeric forms, are contemplated.

Diastereomeric mixtures can be separated into their individual diastereomers on the basis of their physical chemical differences by methods well known to those skilled in the art, such as, for example, by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereomers to the corresponding pure enantiomers. Also, some of the compounds disclosed herein may be atropisomers and are considered as part of this disclosure. Stereoisomers can also be separated by use of chiral HPLC.

Some of the compounds exist as tautomers. Tautomers are in equilibrium with one another. For example, amide containing compounds may exist in equilibrium with imidic acid tautomers. Regardless of which tautomer is shown and regardless of the nature of the equilibrium among tautomers, the compounds are understood by one of ordinary skill in the art to comprise both amide and imidic acid tautomers. Thus, the amide containing compounds are understood to include their imidic acid tautomers. Likewise, the imidic acid containing compounds are understood to include their amide tautomers.

Any compound or structure given herein, is also intended to represent unlabeled forms as well as isotopically labeled forms of the compounds. These forms of compounds may also be referred to as an “isotopically enriched analog.” Isotopically labeled compounds have structures depicted herein, except that one or more atoms are replaced by an atom having a selected atomic mass or mass number. Examples of isotopes that can be incorporated into the disclosed compounds include isotopes of hydrogen, carbon, nitrogen, oxygen, phosphorous, fluorine, chlorine and iodine, such as ²H, ³H, ¹¹C, ¹³C, ¹⁴C, ¹³N, ¹⁵N, ¹⁵O, ¹⁷O, ¹⁸O, ³¹P, ³²P, ³⁵S, ¹⁸F, ³⁶Cl, ¹²³I and ¹²⁵I, respectively. Various isotopically labeled compounds of the present disclosure, for example those into which radioactive isotopes such as ³H and ¹⁴C are incorporated. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients. Such compounds may exhibit increased resistance to metabolism and are thus useful for increasing the half-life of any compound when administered to a mammal, particularly a human. Such compounds are synthesized by means well known in the art, for example by employing starting materials in which one or more hydrogens have been replaced by deuterium.

The terms “inhibit,” “inhibiting,” and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

“Individual” as used herein is a mammal, including humans. In some embodiments, individuals include pig, bovine, feline, canine, primate, rodent, or human. In some embodiments, the individual is human.

As used herein, “treatment” or “treating” is an approach for obtaining beneficial or desired results including clinical results. For purposes of this disclosure, beneficial or desired results include, but are not limited to, one or more of the following: decreasing one or more symptoms resulting from the disease or disorder, diminishing the extent of the disease or disorder, stabilizing the disease or disorder (e.g., preventing or delaying the worsening of the disease or disorder), delaying the occurrence or recurrence of the disease or disorder, delaying or slowing the progression of the disease or disorder, ameliorating the disease or disorder state, providing a remission (whether partial or total) of the disease or disorder, decreasing the dose of one or more other medications required to treat the disease or disorder, enhancing the effect of another medication used to treat the disease or disorder, delaying the progression of the disease or disorder, increasing the quality of life, and/or prolonging survival of a patient. Also encompassed by “treatment” is a reduction of pathological consequence of the disease or disorder. The methods of this disclosure contemplate any one or more of these aspects of treatment.

The term “effective amount” used herein refers to an amount of a compound or composition sufficient to treat a specified disorder, condition or disease such as ameliorate, palliate, lessen, and/or delay one or more of its symptoms. In some embodiments, an effective amount is an amount sufficient to delay development. In some embodiments, an effective amount is an amount sufficient to delay occurrence and/or prevent recurrence. An effective amount can be administered in one or more administrations.

The term “carrier,” as used herein, refers to relatively nontoxic chemical compounds or agents that facilitate the incorporation of a compound into cells or tissues.

As used herein, by “pharmaceutically acceptable” or “pharmacologically acceptable” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

“Pharmaceutically acceptable salts” are those salts which retain at least some of the biological activity of the free (non-salt) compound and which can be administered as drugs or pharmaceuticals to an individual. Such salts, for example, include: (1) acid addition salts, formed with inorganic acids such as hydrochloric acid, hydrobromic acid, sulfuric acid, nitric acid, phosphoric acid and the like; or formed with organic acids such as acetic acid, oxalic acid, propionic acid, succinic acid, maleic acid, tartaric acid and the like; (2) salts formed when an acidic proton present in the parent compound either is replaced by a metal ion, e.g., an alkali metal ion, an alkaline earth ion, or an aluminum ion; or coordinates with an organic base. Acceptable organic bases include ethanolamine, diethanolamine, triethanolamine and the like. Acceptable inorganic bases include aluminum hydroxide, calcium hydroxide, potassium hydroxide, sodium carbonate, sodium hydroxide and the like. Further examples of pharmaceutically acceptable salts include those listed in Berge et al., Pharmaceutical Salts, J. Pharm. Sci. 1977 January; 66(1):1-19. Pharmaceutically acceptable salts can be prepared in situ in the manufacturing process, or by separately reacting a purified compound of the disclosure in its free acid or base form with a suitable organic or inorganic base or acid, respectively and isolating the salt thus formed during subsequent purification.

The term “excipient” as used herein means an inert or inactive substance that may be used in the production of a drug or pharmaceutical, such as a tablet containing a compound of the disclosure as an active ingredient. Various substances may be embraced by the term excipient, including without limitation any substance used as a binder, disintegrant, coating, compression/encapsulation aid, cream or lotion, lubricant, solutions for parenteral administration, materials for chewable tablets, sweetener or flavoring, suspending/gelling agent, or wet granulation agent. Binders include, e.g., carbomers, povidone, xanthan gum, etc.; coatings include, e.g., cellulose acetate phthalate, ethylcellulose, gellan gum, maltodextrin, enteric coatings, etc.; compression/encapsulation aids include, e.g., calcium carbonate, dextrose, fructose dc (dc=“directly compressible”), honey dc, lactose (anhydrate or monohydrate; optionally in combination with aspartame, cellulose, or microcrystalline cellulose), starch dc, sucrose, etc.; disintegrants include, e.g., croscarmellose sodium, gellan gum, sodium starch glycolate, etc.; creams or lotions include, e.g., maltodextrin, carrageenans, etc.; lubricants include, e.g., magnesium stearate, stearic acid, sodium stearyl fumarate, etc.; materials for chewable tablets include, e.g., dextrose, fructose dc, lactose (monohydrate, optionally in combination with aspartame or cellulose), etc.; suspending/gelling agents include, e.g., carrageenan, sodium starch glycolate, xanthan gum, etc.; sweeteners include, e.g., aspartame, dextrose, fructose dc, sorbitol, sucrose dc, etc.; and wet granulation agents include, e.g., calcium carbonate, maltodextrin, microcrystalline cellulose, etc.

### Compounds

In one aspect, provided is a compound of formula (I), or a stereoisomer or a pharmaceutically acceptable salt thereof,

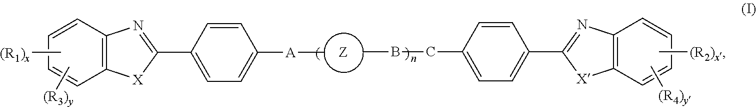

wherein:
x and x′ are each independently 0, 1, 2, 3, or 4;
each of R₁ and R₂ is independently —Rₐ, —N(Rₐ)₂, —ORₐ, —C(O)ORₐ, —OC(O)Rₐ, —NHC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, —NHS(O)₂Rₐ, —NHS(O)₂N(Rₐ)₂, nitro, cyano, or halogen, wherein each Rₐ is independently hydrogen, C₁₋₆ alkyl, C₃₋₈ cycloalkyl, 3-12 membered heterocyclyl, C₆₋₁₂ aryl, or 5-12 membered heteroaryl, and wherein any two of R₁ or any two of R₂ can be taken together with the atoms to which they attach to form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
y and y′ are each independently 0, 1, 2, 3, or 4;
R₃ is

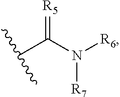

wherein
   R₅ is O, S, or NH, and
   R₆ and R₇ are each independently hydrogen, C₁₋₆ alkyl, C₃₋₈ cycloalkyl, 3-12 membered heterocyclyl, C₆₋₁₂ aryl, 5-12 membered heteroaryl, —C(O)OR_{d} or —S(O)₂R_{d}, wherein each R_{d} is independently hydrogen, C₁₋₁₂ alkyl, C₃₋₈ cycloalkyl, 3-12 membered heterocyclyl, C₆₋₁₂ aryl, or 5-12 membered heteroaryl, and wherein R₆ and R₇ can be taken together with the nitrogen atom to which they attach to form a 3-12 membered heterocyclyl or 5-12 membered heteroaryl, or
   when y is 2, 3, or 4, then two R₃ can be taken together with the atoms to which they attach to form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
R₄ is

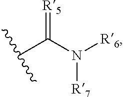

wherein
   R′₅ is O, S, or NH, and
   R′₆ and R′₇ are each independently hydrogen, C₁₋₆ alkyl, C₃₋₈ cycloalkyl, 3-12 membered heterocyclyl, C₆₋₁₂ aryl, 5-12 membered heteroaryl, —C(O)OR_{d} or —S(O)₂R_{d}, wherein each R_{d} is independently hydrogen, C₁₋₁₂ alkyl, C₃₋₈ cycloalkyl, 3-12 membered heterocyclyl, C₆₋₁₂ aryl, or 5-12 membered heteroaryl, and wherein R′₆ and R′₇ can be taken together with the nitrogen atom to which they attach to form a 3-12 membered heterocyclyl or 5-12 membered heteroaryl, or
   when y′ is 2, 3, or 4, then two R₄ can be taken together with the atoms to which they attach for form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
X is O, S, NH, or NRs and X′ is O, S, NH, or NR′₈, wherein
   R₈ and R′₈ are each independently C₁₋₆ alkyl, C₃₋₈ cycloalkyl, 3-12 membered heterocyclyl, C₆₋₁₂ aryl, or 5-12 membered heteroaryl;
A and B are each independently —C(O)—, —C(O)NH—, —NHC(O)—, —S(O)₂—, —S(O)₂NH—, or —NHS(O)₂—;
C is a chemical bond or —NH—, provided that
   when B is —C(O)— or —S(O)₂—, then C is —NH—, and
   when B is —C(O)NH—, —NHC(O)—, —S(O)₂NH—, or —NHS(O)₂—, then C is a chemical bond;
n is an integer selected from 1-6;
each Z is independently C₁₋₆ alkyl, C₃₋₈ cycloalkyl, 3-12 membered heterocyclyl, C₆₋₁₂ aryl, or 5-12 membered heteroaryl, each of which is independently optionally substituted by R_{c}, wherein each R_{c} is independently C₁₋₆ alkyl, C₁₋₆ alkoxyl, C₃₋₈ cycloalkyl, 3-12 membered heterocyclyl, C₆₋₁₂ aryl, 5-12 membered heteroaryl, amino, hydroxyl, carboxyl, nitro, cyano, or halogen;
   provided that at least one Z is C₃₋₈ cycloalkyl, 3-12 membered heterocyclyl, C₆₋₁₂ aryl, or 5-12 membered heteroaryl, each of which is independently optionally substituted by Rₑ; and
each R₉ is independently —R_{b}, —N(R_{b})₂, —OR_{b}, —C(O)OR_{b}, —OC(O)R_{b}, —NHC(O)R_{b}, —C(O)N(R_{b})₂, —OC(O)N(R_{b})₂, —NHC(O)N(R_{b})₂, —S(O)₂R_{b}, —S(O)₂N(R_{b})₂, —C(O)R_{b}, —NHS(O)₂R_{b}, —NHS(O)₂N(R_{b})₂, nitro, cyano, or halogen, wherein each R_{b} is independently hydrogen, C₁₋₆ alkyl, C₃₋₈ cycloalkyl, 3-12 membered heterocyclyl, C₆₋₁₂ aryl, or 5-12 membered heteroaryl.

In some embodiments of compound of formula (I) or any related formula, x is 0, 1, 2, or 3. In some embodiments, x is 0, 1, or 2. In some embodiments, x is 0 or 1. In some embodiments, x is 1, 2, or 3. In some embodiments, x is 1 or 2. In some embodiments, x is 2 or 3. In some embodiments, x is 0. In some embodiments, x is 1. In some embodiments, x is 2. In some embodiments, x is 3. In some embodiments, x is 4.

In some embodiments of compound of formula (I) or any related formula, x′ is 0, 1, 2, or 3. In some embodiments, x′ is 0, 1, or 2. In some embodiments, x′ is 0 or 1. In some embodiments, x′ is 1, 2, or 3. In some embodiments, x′ is 1 or 2. In some embodiments, x′ is 2 or 3. In some embodiments, x′ is 0. In some embodiments, x′ is 1. In some embodiments, x′ is 2. In some embodiments, x′ is 3. In some embodiments, x′ is 4.

In some embodiments of compound of formula (I) or any related formula, x is equal to x′. In some embodiments, x is equal to x′ and is 0. In some embodiments, x is equal to x′ and is 1. In some embodiments, x is equal to x′ and is 2. In some embodiments, x is equal to x′ and is 3. In some embodiments, x is equal to x′ and is 4. In some embodiments, x and x′ are each independently 2 or 3. In some embodiments, x is equal to x′ and is 2 or 3.

In some embodiments of compound of formula (I) or any related formula, each R₁ is independently —Rₐ, —ORₐ, or halogen. In some embodiments, each R₁ is independently —Rₐ, —ORₐ, or halogen, wherein each Rₐ is independently hydrogen or C₁₋₆ alkyl. In some embodiments, each R₁ is independently hydrogen, methyl, methoxyl, or fluoro. In some embodiments, R₁ is hydrogen. In some embodiments, R₁ is C₁₋₆ alkyl. In some embodiments, R₁ is methyl. In some embodiments, R₁ is —O—C₁₋₆ alkyl. In some embodiments, R₁ is methoxyl. In some embodiments, R₁ is halogen. In some embodiments, R₁ is fluoro. some embodiments, two R₁ are taken together with the atoms to which they attach to form a C₃₋₈ cycloalkyl, 3-12 membered heterocyclyl, C₆₋₁₂ aryl, or 5-12 membered heteroaryl, each of which is independently optionally substituted by R₉. In some embodiments, two R₁ are taken together with the atoms to which they attach to form a C₃₋₈ cycloalkyl, which is optionally substituted by R₉. In some embodiments, two R₁ are taken together with the atoms to which they attach to form a 3-12 membered heterocyclyl, which is optionally substituted by R₉. In some embodiments, two R₁ are taken together with the atoms to which they attach to form a C₆₋₁₂ aryl, which is optionally substituted by R. In some embodiments, two R are taken together with the atoms to which they attach to form a 5-12 membered heteroaryl, which is optionally substituted by R₉.

In some embodiments of compound of formula (I) or any related formula, each R₂ is independently —Rₐ, —ORₐ, or halogen. In some embodiments, each R₂ is independently —Rₐ, —ORₐ, or halogen, wherein each Rₐ is independently hydrogen or C₁₋₆ alkyl. In some embodiments, each R₂ is independently hydrogen, methyl, methoxyl, or fluoro. In some embodiments, R₂ is hydrogen. In some embodiments, R₂ is C₁₋₆ alkyl. In some embodiments, R₂ is methyl. In some embodiments, R₂ is —O—C₁₋₆ alkyl. In some embodiments, R₂ is methoxyl. In some embodiments, R₂ is halogen. In some embodiments, R₂ is fluoro. In some embodiments, two R₂ are taken together with the atoms to which they attach to form a C₃₋₈ cycloalkyl, which is optionally substituted by R₉. In some embodiments, two R₂ are taken together with the atoms to which they attach to form a 3-12 membered heterocyclyl, which is optionally substituted by R₉. In some embodiments, two R₂ are taken together with the atoms to which they attach to form a C₆₋₁₂ aryl, which is optionally substituted by R₉. In some embodiments, two R₂ are taken together with the atoms to which they attach to form a 5-12 membered heteroaryl, which is optionally substituted by R₉.

In some embodiments of compound of formula (I) or any related formula, each of R₁ and R₂ is independently —R₈, —ORₐ, or halogen. In some embodiments, each of R₁ and R₂ is independently —Rₐ, —ORₐ, or halogen, wherein each R₈ is independently hydrogen or C₁₋₆ alkyl. In some embodiments, each of R₁ and R₂ is independently hydrogen, methyl, methoxyl, or fluoro. In some embodiments, each of R₁ and R₂ is hydrogen. In some embodiments, two R₁ and/or two R₂ are taken together with the atoms to which they attach to form a C₃₋₈ cycloalkyl, 3-12 membered heterocyclyl, C₆₋₁₂ aryl, or 5-12 membered heteroaryl, each of which is independently optionally substituted by R₉.

In some embodiments of compound of formula (I) or any related formula, y is 0, 1, 2, or 3. In some embodiments, y is 0, 1, or 2. In some embodiments, y is 0 or 1. In some embodiments, y is 1, 2, or 3. In some embodiments, y is 1 or 2. In some embodiments, y is 2 or 3. In some embodiments, y is 0. In some embodiments, y is 1. In some embodiments, y is 2. In some embodiments, y is 3. In some embodiments, y is 4.

In some embodiments of compound of formula (I) or any related formula, y′ is 0, 1, 2, or 3. In some embodiments, y′ is 0, 1, or 2. In some embodiments, y′ is 0 or 1. In some embodiments, y′ is 1, 2, or 3. In some embodiments, y′ is 1 or 2. In some embodiments, y′ is 2 or 3. In some embodiments, y′ is 0. In some embodiments, y′ is 1. In some embodiments, y′ is 2. In some embodiments, y′ is 3. In some embodiments, y′ is 4.

In some embodiments of compound of formula (I) or any related formula, y is equal to y′. In some embodiments, y is equal to y′ and is 0. In some embodiments, y is equal to y′ and is 1. In some embodiments, y is equal to y′ and is 2. In some embodiments, y is equal to y′ and is 3. In some embodiments, y is equal to y′ and is 4. In some embodiments, y and y′ are each independently 1 or 2. In some embodiments, y is equal to y′ and is 1 or 2. In some embodiments, x+y and x′+y′ are equal to 4.

In some embodiments of compound of formula (I) or any related formula, R₅ is O. In some embodiments, R₅ is S. In some embodiments, R₅ is NH. In some embodiments, R₅ is O or NH.

In some embodiments of compound of formula (I) or any related formula, R₆ and R₇ are each independently hydrogen or —C(O)OR_{d}. In some embodiments, R₆ and R₇ are each independently hydrogen or —C(O)OR_{d}, wherein R_{d} is C₁₋₁₂ alkyl. In some embodiments, R₆ and R₇ are both hydrogen. In some embodiments, R₆ and R₇ can be taken together with the nitrogen atom to which they attach to form a 3-12 membered heterocyclyl or 5-12 membered heteroaryl.

In some embodiments of compound of formula (I) or any related formula, R₅ is O; and R₆ and R₇ are each independently hydrogen or —C(O)OR_{d}, wherein R_{d} is C₁₋₁₂ alkyl. In some embodiments, R₅ is S; and R₆ and R₇ are each independently hydrogen or —C(O)OR_{d}, wherein R_{d} is C₁₋₁₂ alkyl. In some embodiments, R₅ is NH; and R₆ and R₇ are each independently hydrogen or —C(O)OR_{d}, wherein R_{d} is C₁₋₁₂ alkyl. In some embodiments, R₅ is O or NH; and R₆ and R₇ are each independently hydrogen or —C(O)OR_{d}, wherein R_{d} is C₁₋₁₂ alkyl. In some embodiments, R₅ is O; and R₆ and R₇ are both hydrogen. In some embodiments, R₅ is NH; and R₆ and R₇ are both hydrogen. In some embodiments, R₅ is S; and R₆ and R₇ are both hydrogen.

In some embodiments of compound of formula (I) or any related formula, R′₅ is O. In some embodiments, R′₅ is S. In some embodiments, R′₅ is NH. In some embodiments, R′₅ is O or NH.

In some embodiments of compound of formula (I) or any related formula, R′₆ and R′₇ are each independently hydrogen or —C(O)OR_{d}. In some embodiments, R′₆ and R′₇ are each independently hydrogen or —C(O)OR_{d}, wherein Rₐ is C₁₋₁₂ alkyl. In some embodiments. R′₆ and R′₇ are both hydrogen. In some embodiments, R′₆ and R′₇ can be taken together with the nitrogen atom to which they attach to form a 3-12 membered heterocyclyl or 5-12 membered heteroaryl.

In some embodiments of compound of formula (I) or any related formula, R′₅ is O; and R′₆ and R′₇ are each independently hydrogen or —C(O)OR_{d}, wherein R_{d} is C₁₋₁₂ alkyl. In some embodiments, R′₅ is S; and R′₆ and R′₇ are each independently hydrogen or —C(O)OR_{d}, wherein R_{d} is C₁₋₁₂ alkyl. In some embodiments, R′₅ is NH; and R′₆ and R′₇ are each independently hydrogen or —C(O)OR_{d}, wherein R_{d} is C₁₋₁₂ alkyl. In some embodiments, R′₅ is O or NH; and R′₆ and R′₇ are each independently hydrogen or —C(O)OR_{d}, wherein R_{d} is C₁₋₁₂ alkyl. In some embodiments, R's is O; and R′₆ and R′₇ are both hydrogen. In some embodiments, R′₅ is NH; and R′₆ and R′₇ are both hydrogen. In some embodiments, R′₅ is S; and R′₆ and R′₇ are both hydrogen.

In some embodiments of compound of formula (I) or any related formula, two R₃ are taken together with the atoms to which they attach to form C₃₋₈ cycloalkyl, 3-12 membered heterocyclyl, C₆₋₁₂ aryl, or 5-12 membered heteroaryl, each of which is independently optionally substituted by R₉. In some embodiments, two R₃ are taken together with the atoms to which they attach to form C₃₋₈ cycloalkyl, which is optionally substituted by R₉. In some embodiments, two R₃ are taken together with the atoms to which they attach to form 3-12 membered heterocyclyl, which is optionally substituted by R₉. In some embodiments, two R₃ are taken together with the atoms to which they attach to form C₆₋₁₂ aryl, which is optionally substituted by R₉. In some embodiments, two R₃ are taken together with the atoms to which they attach to form 5-12 membered heteroaryl, which is optionally substituted by R₉. In some embodiments, two R₃ are taken together with the atoms to which they attach to form 5 or 6 membered heteroaryl, which is optionally substituted by R₉. In some embodiments, two R₃ are taken together with the atoms to which they attach to form

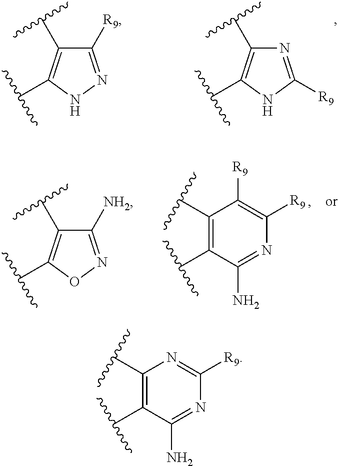

embodiments, two R₃ are taken together with the atoms to which they attach to form

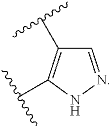 [SMILES: *c1cn[nH]c1*]

In some embodiments of compound of formula (I) or any related formula, two R₄ are taken together with the atoms to which they attach to form C₃₋₈ cycloalkyl, 3-12 membered heterocyclyl, C₆₋₁₂ aryl, or 5-12 membered heteroaryl, each of which is independently optionally substituted by R₉. In some embodiments, two R₄ are taken together with the atoms to which they attach to form C₃-s cycloalkyl, which is optionally substituted by R₉. In some embodiments, two Rₐ are taken together with the atoms to which they attach to form 3-12 membered heterocyclyl, which is optionally substituted by R₉. In some embodiments, two R₄ are taken together with the atoms to which they attach to form C₆₋₁₂ aryl, which is optionally substituted by R₉. In some embodiments, two R₄ are taken together with the atoms to which they attach to form 5-12 membered heteroaryl, which is optionally substituted by R₉. In some embodiments, two R₄ are taken together with the atoms to which they attach to form 5 or 6 membered heteroaryl, which is optionally substituted by R₉. In some embodiments, two R₄ are taken together with the atoms to which they attach to form

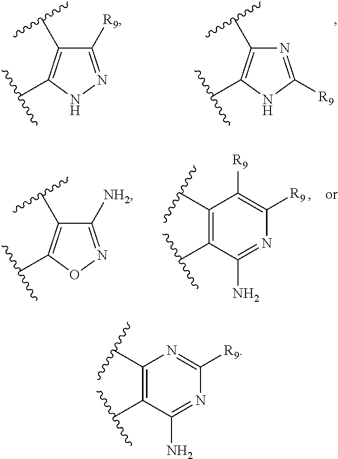

In some embodiments, two R₄ are taken together with the atoms to which they attach to form

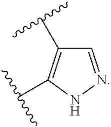 [SMILES: *c1cn[nH]c1*]

In some embodiments of compound of formula (I) or any related formula, two R₃ and/or two R₄ are taken together with the atoms to which they attach to form C₃₋₈ cycloalkyl, 3-12 membered heterocyclyl, C₆₋₁₂ aryl, or 5-12 membered heteroaryl, each of which is independently optionally substituted by R₉. In some embodiments, two R₃ and/or two R₄ are taken together with the atoms to which they attach to form 5-12 membered heteroaryl, which is optionally substituted by R₉. In some embodiments, two R₃ and/or two R₄ are taken together with the atoms to which they attach to form 5 or 6 membered heteroaryl, each of which is optionally substituted by R₉. In some embodiments, two R₃ and/or two R₄ are taken together with the atoms to which they attach to form

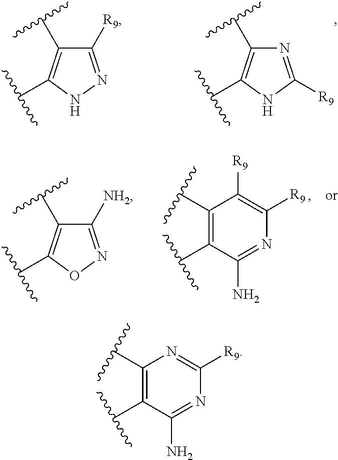

In some embodiments, two R₃ and/or two R₄ are taken together with the atoms to which they attach to form

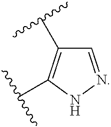 [SMILES: *c1cn[nH]c1*]

In some embodiments of compound of formula (I) or any related formula, X is O. In some embodiments, X is S. In some embodiments, X is NH. In some embodiments, X is NRs. In some embodiments, X is NH or NRs. In some embodiments, R₈ is C₁₋₆ alkyl. In some embodiments, R₈ is methyl.

In some embodiments of compound of formula (I) or any related formula, X′ is O. In some embodiments, X′ is S. In some embodiments, X′ is NH. In some embodiments, X′ is NR′₈. In some embodiments, X′ is NH or NR′₈. In some embodiments, R′₈ is C₁₋₆ alkyl. In some embodiments, R′₈ is methyl.

In some embodiments of compound of formula (I) or any related formula, X is NH or NR₈ and X′ is NH or NR′₈, wherein R₈ and R′₈ are each independently C₁₋₆ alkyl. In some embodiments, X is NH or NR₈ and X′ is NH or NR′₈, wherein R₈ and R′₈ are both methyl. In some embodiments, X and X′ are both NH.

In some embodiments of compound of formula (I) or any related formula, A is —C(O)—, —C(O)NH—, —NHC(O)—, —S(O)₂—, —S(O)₂NH—, or —NHS(O)₂—. In some embodiments, A is —C(O)—, —C(O)NH—, or —NHC(O)—. In some embodiments, A is —C(O)—. In some embodiments, A is —C(O)NH—. In some embodiments, A is —NHC(O)—. In some embodiments, A is —S(O)₂—. In some embodiments, A is —S(O)₂NH—. In some embodiments, A is —NHS(O)₂—.

In some embodiments of compound of formula (I) or any related formula, B is —C(O)—, —C(O)NH—, —NHC(O)—, —S(O)₂—, —S(O)₂NH—, or —NHS(O)₂—. In some embodiments, B is —C(O)—, —C(O)NH—, or —NHC(O)—. In some embodiments, B is —C(O)—. In some embodiments, B is —C(O)NH—. In some embodiments, B is —NHC(O)—. In some embodiments, B is —S(O)₂—. In some embodiments. B is —S(O)₂NH—. In some embodiments, B is —NHS(O)₂—.

In some embodiments of compound of formula (I) or any related formula, A and B are each independently —C(O)—, —C(O)NH—, or —NHC(O)—. In some embodiments, each of A and B is —C(O)—.

In some embodiments of compound of formula (I) or any related formula, n is 1. In some embodiments, n is 2. In some embodiments, n is 3. In some embodiments, n is 4. In some embodiments, n is 5. In some embodiments, n is 6. In some embodiments, n is 2-6. In some embodiments, n is 2-5. In some embodiments, n is 2-4. In some embodiments, n is 2-3. In some embodiments, n is 3-6. In some embodiments, n is 3-5. In some embodiments, n is 3-4. In some embodiments, n is 4-6. In some embodiments, n is 4-5.

In some embodiments of compound of formula (I) or any related formula, each Z is independently C₁₋₆ alkyl, 3-12 membered heterocyclyl, or 5-12 membered heteroaryl, each of which is independently optionally substituted by Rₑ. In some embodiments, each Z is independently 3-12 membered heterocyclyl, which is optionally substituted by Rₑ. In some embodiments, each Z is independently C₃-s cycloalkyl, which is optionally substituted by Rₑ. In some embodiments, each Z is independently C₆₋₁₂ aryl, which is optionally substituted by R_{c}. In some embodiments, each Z is independently 5-12 membered heteroaryl, which is optionally substituted by Rₑ. In some embodiments, each Z is independently —CH₂—, —CH₂CH₂—,

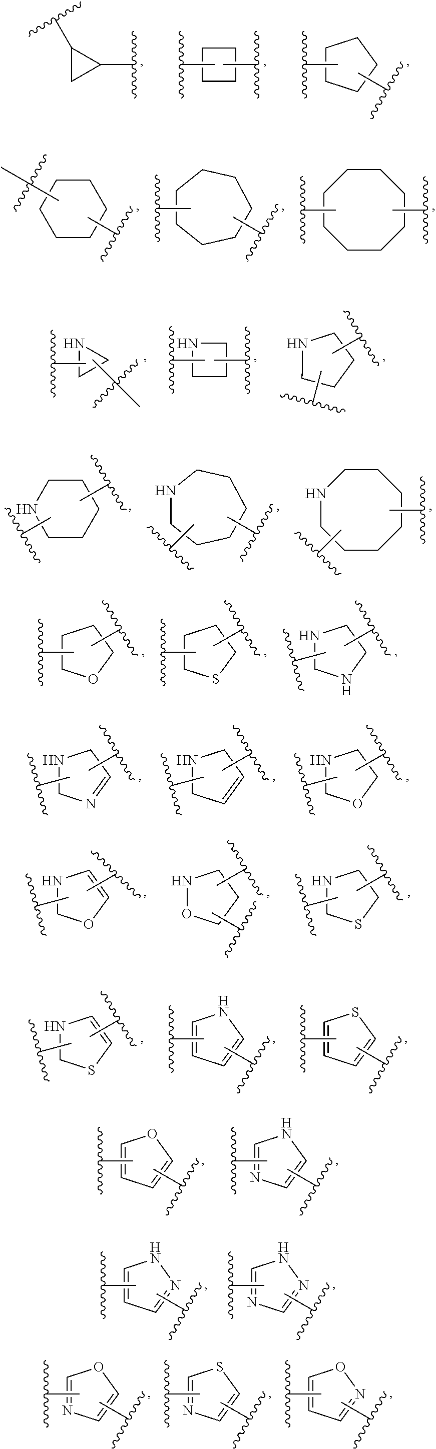

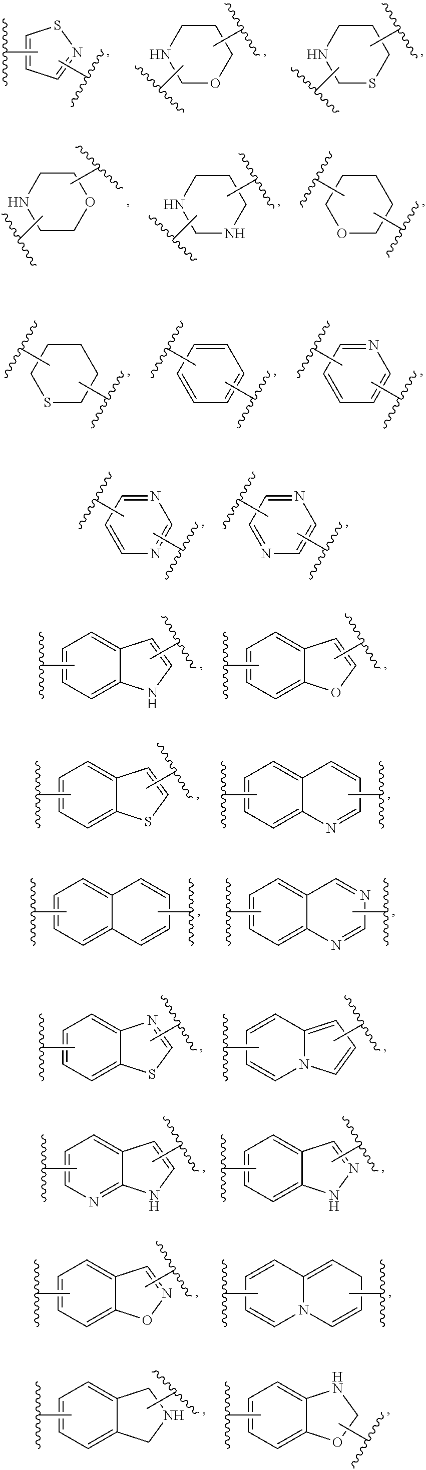

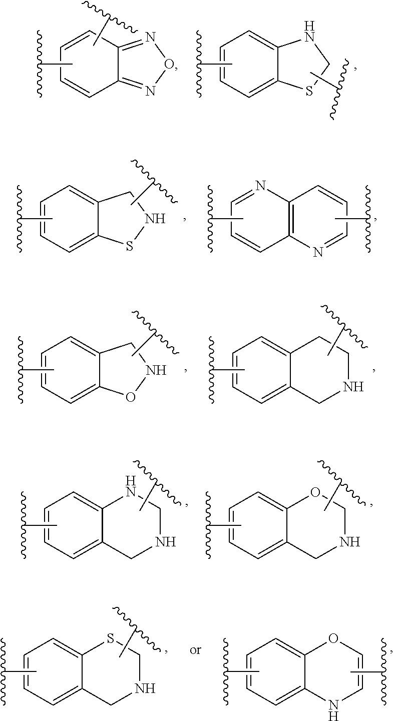

each of which is independently optionally substituted by R_{c}, It is understood that each wavy line indicates the point of attachment to the rest of the molecule and the point of attachment can be at any atom as valency permits. For example,

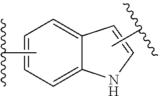

contemplates, without limitation,

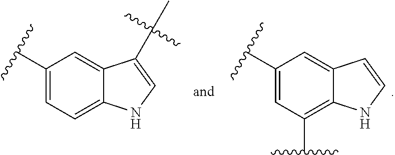 [SMILES: *c1ccc2[nH]cc(*)c2c1 ; *c1cc(*)c2[nH]ccc2c1]

In some embodiments, each Z is independently methyl,

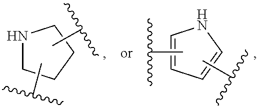

each of which is independently optionally substituted by R_{c}. In some embodiments, each Z is independently

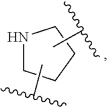

which is optionally substituted by R. In some embodiments, each Z is

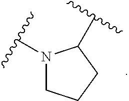 [SMILES: *C1CCCN1*]

In some embodiments, each Z is independently

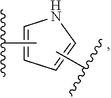

(which is optionally substituted by R_{c}. In some embodiments, each Z is independently

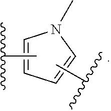

It is understood that specific values described herein are values for a compound of formula (I) or any related formula where applicable, such as formula (II). Two or more values may combined. Thus, it is to be understood that any variable for a compound of formula (I) or any related formula may be combined with any other variable for a compound of formula (I) or any related formula the same as if each and every combination of variables were specifically and individually listed. As an example, in some embodiments, provided is a compound of formula (I), or a stereoisomer or a pharmaceutically acceptable salt thereof, wherein x and x′ are each 2 or 3; each of R₁ and R₂ is hydrogen; y and y′ are each independently 1 or 2, wherein two R₃ and/or two R₄ can be taken together with the atoms to which they attach to form

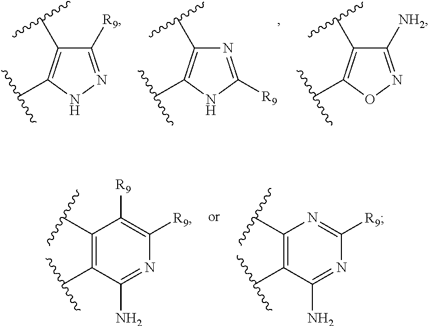

R₅ is O or NH; R₆ and R₇ are each independently hydrogen or —C(O)ORₐ; R′₅ is O or NH; R′₆ and R′₇ are each independently hydrogen or —C(O)ORₐ; X is NH or NR₈ and X′ is NH or NR′₈, wherein R₈ and R′₈ are each independently C₁₋₆ alkyl; A and B are each independently —C(O)—, —C(O)NH—, or —NHC(O)—; n is 2; and each Z is independently C₁₋₆ alkyl, 3-12 membered heterocyclyl, or 5-12 membered heteroaryl, each of which is independently optionally substituted by Rₑ.

Exemplary compounds provided by the present disclosure include, but are not limited to, a compound, shown in Table 1, or a stereoisomer, tautomer, hydrate, solvate, isotopically labeled form, or pharmaceutically acceptable salt thereof. In some embodiments, provided is a compound shown in Table 1, or a stereoisomer or pharmaceutically acceptable salt thereof. In some embodiments, provided is a compound shown in Table 1 or pharmaceutically acceptable salt thereof.

TABLE 1

| | |
|---|---|
| 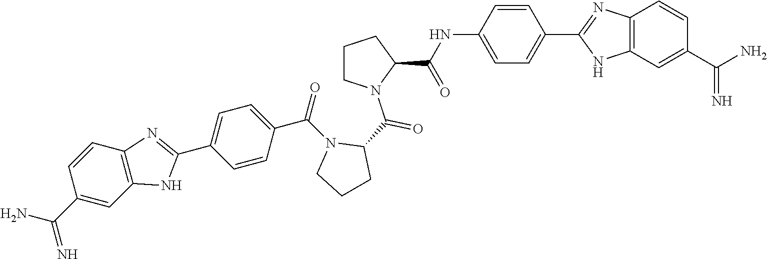 [SMILES: N=C(N)c1ccc2nc(-c3ccc(NC(=O)[C@@H]4CCCN4C(=O)[C@@H]4CCCN4C(=O)c4ccc(-c5nc6ccc(C(=N)N)cc6[nH]5)cc4)cc3)[nH]c2c1] | I-1 |
| 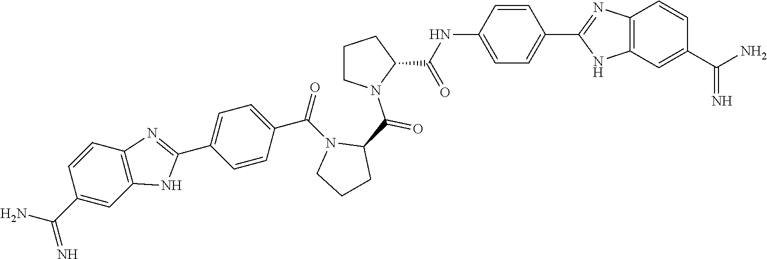 [SMILES: N=C(N)c1ccc2nc(-c3ccc(NC(=O)[C@H]4CCCN4C(=O)[C@H]4CCCN4C(=O)c4ccc(-c5nc6ccc(C(=N)N)cc6[nH]5)cc4)cc3)[nH]c2c1] | I-2 |
| 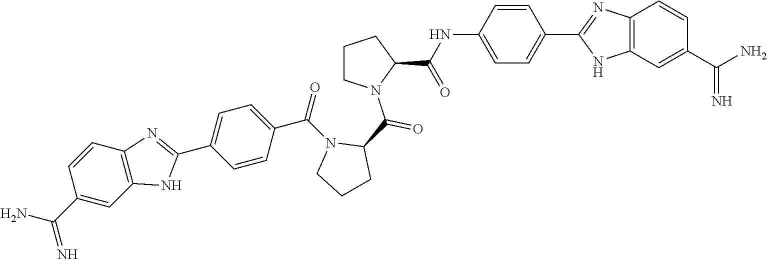 [SMILES: N=C(N)c1ccc2nc(-c3ccc(NC(=O)[C@@H]4CCCN4C(=O)[C@H]4CCCN4C(=O)c4ccc(-c5nc6ccc(C(=N)N)cc6[nH]5)cc4)cc3)[nH]c2c1] | I-3 |
| 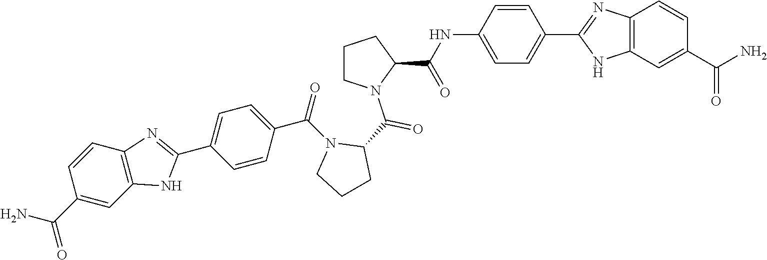 [SMILES: NC(=O)c1ccc2nc(-c3ccc(NC(=O)[C@@H]4CCCN4C(=O)[C@@H]4CCCN4C(=O)c4ccc(-c5nc6ccc(C(N)=O)cc6[nH]5)cc4)cc3)[nH]c2c1] | I-4 |
| 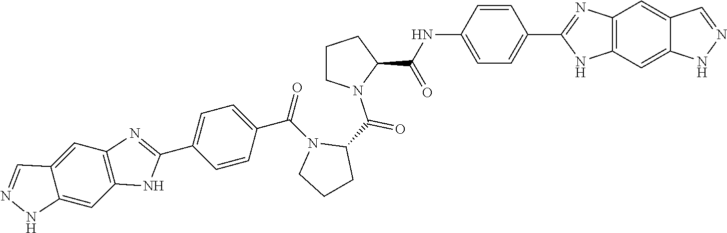 [SMILES: O=C(Nc1ccc(-c2nc3cc4cn[nH]c4cc3[nH]2)cc1)[C@@H]1CCCN1C(=O)[C@@H]1CCCN1C(=O)c1ccc(-c2nc3cc4cn[nH]c4cc3[nH]2)cc1] | I-5 |
| 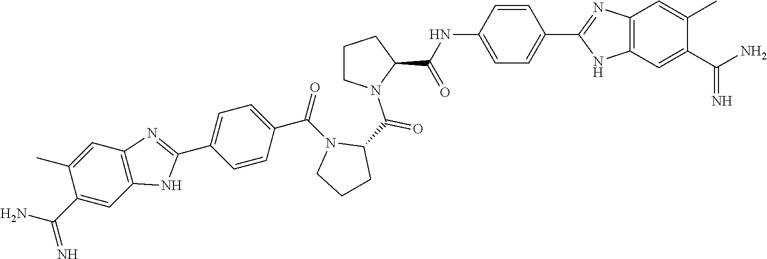 [SMILES: Cc1cc2nc(-c3ccc(NC(=O)[C@@H]4CCCN4C(=O)[C@@H]4CCCN4C(=O)c4ccc(-c5nc6cc(C)c(C(=N)N)cc6[nH]5)cc4)cc3)[nH]c2cc1C(=N)N] | I-6 |
| 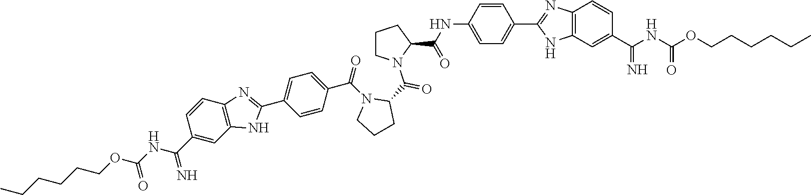 [SMILES: CCCCCCOC(=O)NC(=N)c1ccc2nc(-c3ccc(NC(=O)[C@@H]4CCCN4C(=O)[C@@H]4CCCN4C(=O)c4ccc(-c5nc6ccc(C(=N)NC(=O)OCCCCCC)cc6[nH]5)cc4)cc3)[nH]c2c1] | I-7 |
| 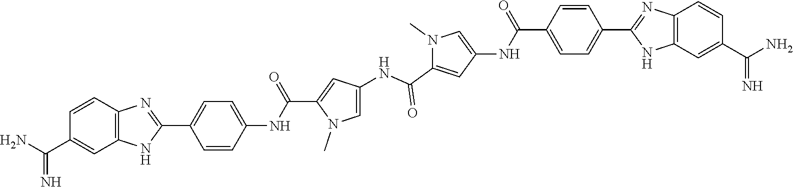 [SMILES: Cn1cc(NC(=O)c2ccc(-c3nc4ccc(C(=N)N)cc4[nH]3)cc2)cc1C(=O)Nc1cc(C(=O)Nc2ccc(-c3nc4ccc(C(=N)N)cc4[nH]3)cc2)n(C)c1] | I-8 |
| 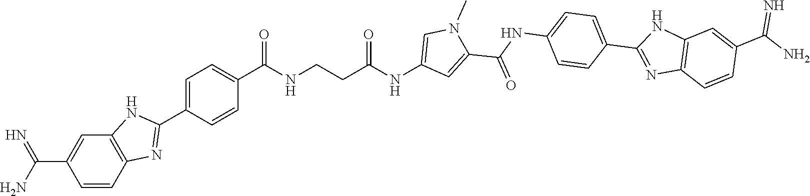 [SMILES: Cn1cc(NC(=O)CCNC(=O)c2ccc(-c3nc4ccc(C(=N)N)cc4[nH]3)cc2)cc1C(=O)Nc1ccc(-c2nc3ccc(C(=N)N)cc3[nH]2)cc1] | I-9 |
| 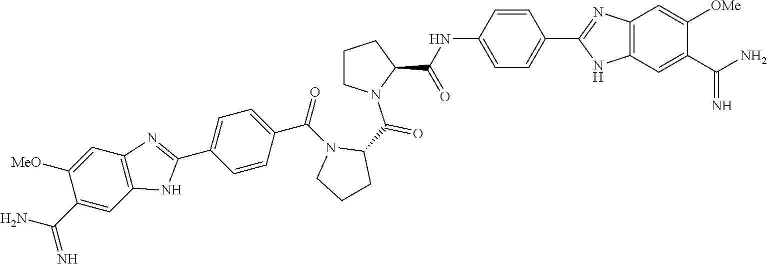 [SMILES: COc1cc2nc(-c3ccc(NC(=O)[C@@H]4CCCN4C(=O)[C@@H]4CCCN4C(=O)c4ccc(-c5nc6cc(OC)c(C(=N)N)cc6[nH]5)cc4)cc3)[nH]c2cc1C(=N)N] | I-10 |
| 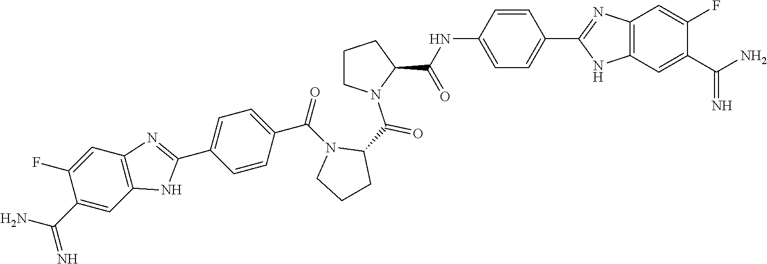 [SMILES: N=C(N)c1cc2[nH]c(-c3ccc(NC(=O)[C@@H]4CCCN4C(=O)[C@@H]4CCCN4C(=O)c4ccc(-c5nc6cc(F)c(C(=N)N)cc6[nH]5)cc4)cc3)nc2cc1F] | I-11 |
| 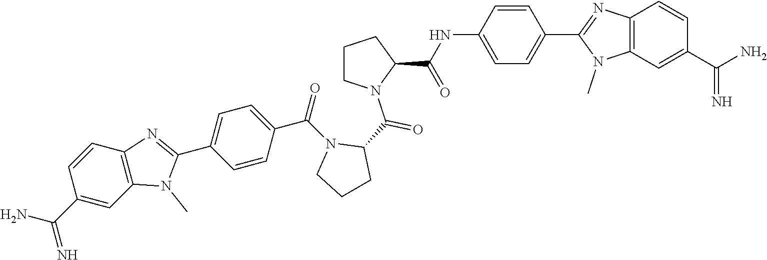 [SMILES: Cn1c(-c2ccc(NC(=O)[C@@H]3CCCN3C(=O)[C@@H]3CCCN3C(=O)c3ccc(-c4nc5ccc(C(=N)N)cc5n4C)cc3)cc2)nc2ccc(C(=N)N)cc21] | I-12 |

### Methods of Treatment

In another aspect, provided is a method of treating a PU.1-mediated disease in an individual in need thereof, comprising administering an effective amount of the compound as described herein, or a stereoisomer or a pharmaceutically acceptable salt thereof, to the individual. Also provided is a compound as described herein, or a stereoisomer or a pharmaceutically acceptable salt thereof, for use in treating a PU.1-mediated disease. In some embodiments, provided is use of a compound as described herein, or a stereoisomer or a pharmaceutically acceptable salt thereof, in the manufacture of a medicament for the treatment of a PU.1-mediated disease. In some embodiments, the PU.1-mediated disease is leukemia or fibrosis. In some embodiments, the PU.1-mediated disease is acute lymphoblastic leukemia (ALL), acute myeloid leukemia (AML), chronic lymphoblastic leukemia (CLL), chronic myeloid leukemia (CML), skin fibrosis, pulmonary fibrosis, renal fibrosis, liver fibrosis, or cardiac fibrosis. In some embodiments, the PU.1-mediated disease is NASH.

In some embodiments, provided is a method of inhibiting PU.1, comprising contacting a cell with an effective amount of a compound disclosed herein, or a stereoisomer or a pharmaceutically acceptable salt thereof.

### Compositions

In another aspect, provided is a composition, such as a pharmaceutical composition, comprising a compound as described herein, or a stereoisomer or a pharmaceutically acceptable salt thereof, and a pharmaceutically acceptable carrier or excipient. The pharmaceutical composition provided herein may take a form suitable for oral, buccal, parenteral (e.g., intravenous, intramuscular, infusion or subcutaneous injection), nasal, topical or rectal administration, or a form suitable for administration by inhalation.

A compound as described herein may, in some embodiments, be in a purified form. In some embodiments, a composition comprising a compound as described herein, or a stereoisomer or a pharmaceutically acceptable salt thereof, is in substantially pure form. Unless otherwise stated, “substantially pure” refers to a composition which contains no more than 35%, 30%, 25%, 20%, 15%, 10%, 5%, 1%, 0.5%, or 0.1% impurity, wherein the impurity denotes a compound other than the desired compound, or a pharmaceutically acceptable salt thereof.

### Kits

Also provided herein is a kit comprising a compound disclosed herein, or a stereoisomer or a pharmaceutically acceptable salt thereof, or a composition disclosed herein. In some embodiments, the kit comprises a unit dose of a compound or composition described herein and/or instructions for administering the same.

### Methods of Preparation

In another aspect, provided is a method of preparing a compound disclosed herein, or a stereoisomer or a pharmaceutically acceptable salt thereof, comprising converting a compound of formula (II),

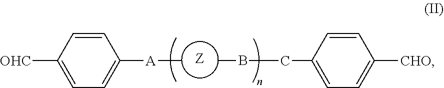

or a stereoisomer or a pharmaceutically acceptable salt thereof, to the compound of formula (I),

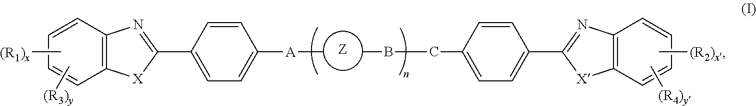

or a stereoisomer or a pharmaceutically acceptable salt thereof, wherein X, X′, x, x′, y, y′, R₁, R₂, R₃, R₄, A, Z, B, C, and n are as disclosed herein.

In some embodiments, the compound of formula (II) is of formula (13′)

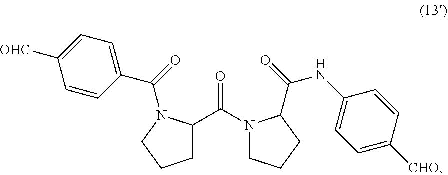 [SMILES: O=Cc1ccc(NC(=O)C2CCCN2C(=O)C2CCCN2C(=O)c2ccc(C=O)cc2)cc1]

or a stereoisomer or a pharmaceutically acceptable salt thereof, and the method further comprises:
(a) reacting a compound of formula (11′),

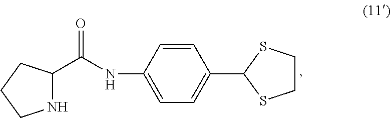 [SMILES: O=C(Nc1ccc(C2SCCS2)cc1)C1CCCN1]

or a stereoisomer or a pharmaceutically acceptable salt thereof, with a compound of formula (5′),

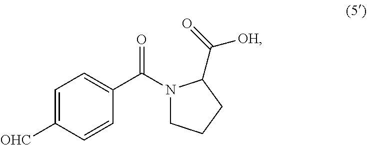 [SMILES: O=Cc1ccc(C(=O)N2CCCC2C(=O)O)cc1]

or a stereoisomer or a pharmaceutically acceptable salt thereof;
(b) converting a compound of formula (6),

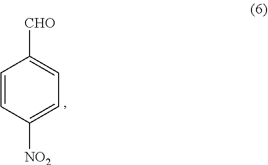 [SMILES: O=Cc1ccc([N+](=O)[O-])cc1]

or a stereoisomer or a pharmaceutically acceptable salt thereof, to the compound of formula (11′), or a stereoisomer or a pharmaceutically acceptable salt thereof; and/or
(c) converting a compound of formula (1),

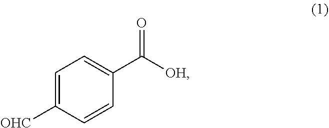 [SMILES: O=Cc1ccc(C(=O)O)cc1]

or a stereoisomer or a pharmaceutically acceptable salt thereof, to the compound of formula (5′), or a stereoisomer or a pharmaceutically acceptable salt thereof.

In some embodiments, the compound of formula (II) is of formula (50),

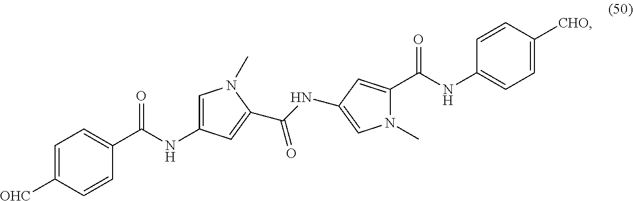 [SMILES: Cn1cc(NC(=O)c2ccc(C=O)cc2)cc1C(=O)Nc1cc(C(=O)Nc2ccc(C=O)cc2)n(C)c1]

or a stereoisomer or a pharmaceutically acceptable salt thereof, and the method further comprises:
(a) reacting a compound of formula (45),

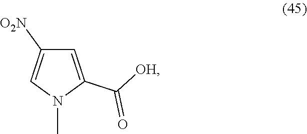

or a stereoisomer or a pharmaceutically acceptable salt thereof, with a compound of formula (41),

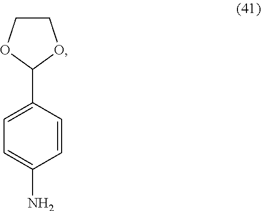 [SMILES: Nc1ccc(C2OCCO2)cc1]

or a stereoisomer or a pharmaceutically acceptable salt thereof;
(b) converting a compound formula (42)

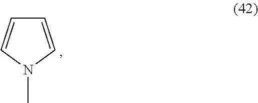

or a stereoisomer or a pharmaceutically acceptable salt thereof, to the compound of formula (45), or a stereoisomer or a pharmaceutically acceptable salt thereof; and/or
(c) converting a compound of formula (6),

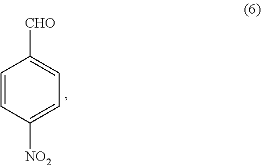 [SMILES: O=Cc1ccc([N+](=O)[O-])cc1]

or a stereoisomer or a pharmaceutically acceptable salt thereof, to the compound of formula (41), or a stereoisomer or a pharmaceutically acceptable salt thereof.

In some embodiments, the compound of formula (II) is of formula (54),

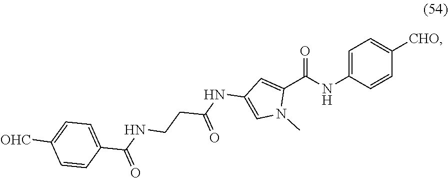 [SMILES: Cn1cc(NC(=O)CCNC(=O)c2ccc(C=O)cc2)cc1C(=O)Nc1ccc(C=O)cc1]

or a stereoisomer or a pharmaceutically acceptable salt thereof, and the method further comprises:
(a) converting a compound of formula (53),

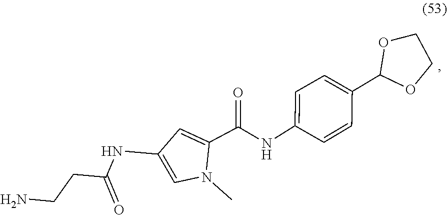 [SMILES: Cn1cc(NC(=O)CCN)cc1C(=O)Nc1ccc(C2OCCO2)cc1]

or a stereoisomer or a pharmaceutically acceptable salt thereof, to the compound of formula (54), or a stereoisomer or a pharmaceutically acceptable salt thereof;
(b) converting a compound of formula (47),

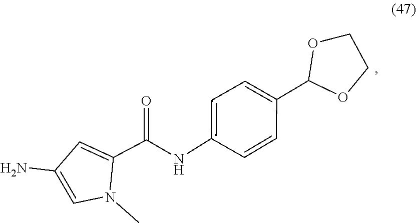 [SMILES: Cn1cc(N)cc1C(=O)Nc1ccc(C2OCCO2)cc1]

or a stereoisomer or a pharmaceutically acceptable salt thereof, to the compound of formula (53), or a stereoisomer or a pharmaceutically acceptable salt thereof; and/or
(c) converting a compound of formula (45),

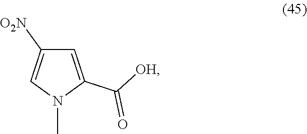

or a stereoisomer or a pharmaceutically acceptable salt thereof, to the compound of formula (47), or a stereoisomer or a pharmaceutically acceptable salt thereof.

In some embodiments, one or more steps of a preparation method disclosed herein comprise acylation, condensation, reduction, protection, and/or deprotection.

Representative schemes for preparing the compounds disclosed herein are provided below.

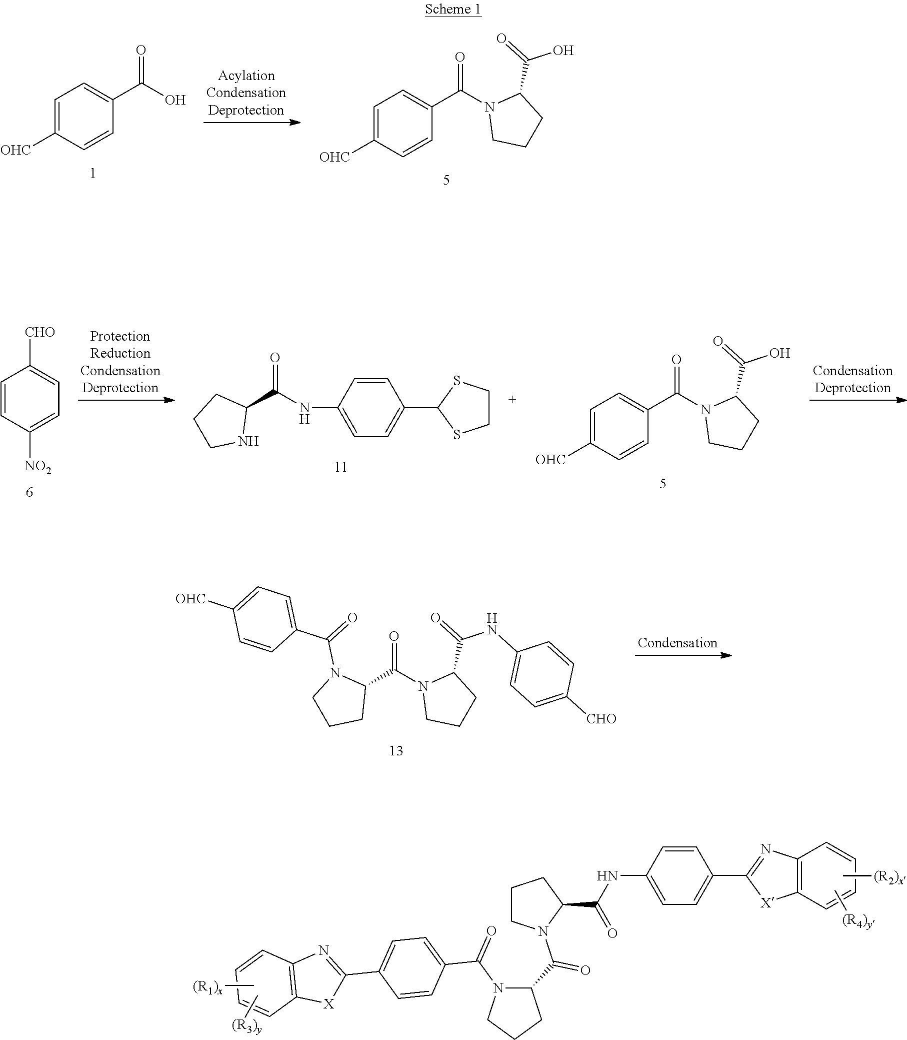

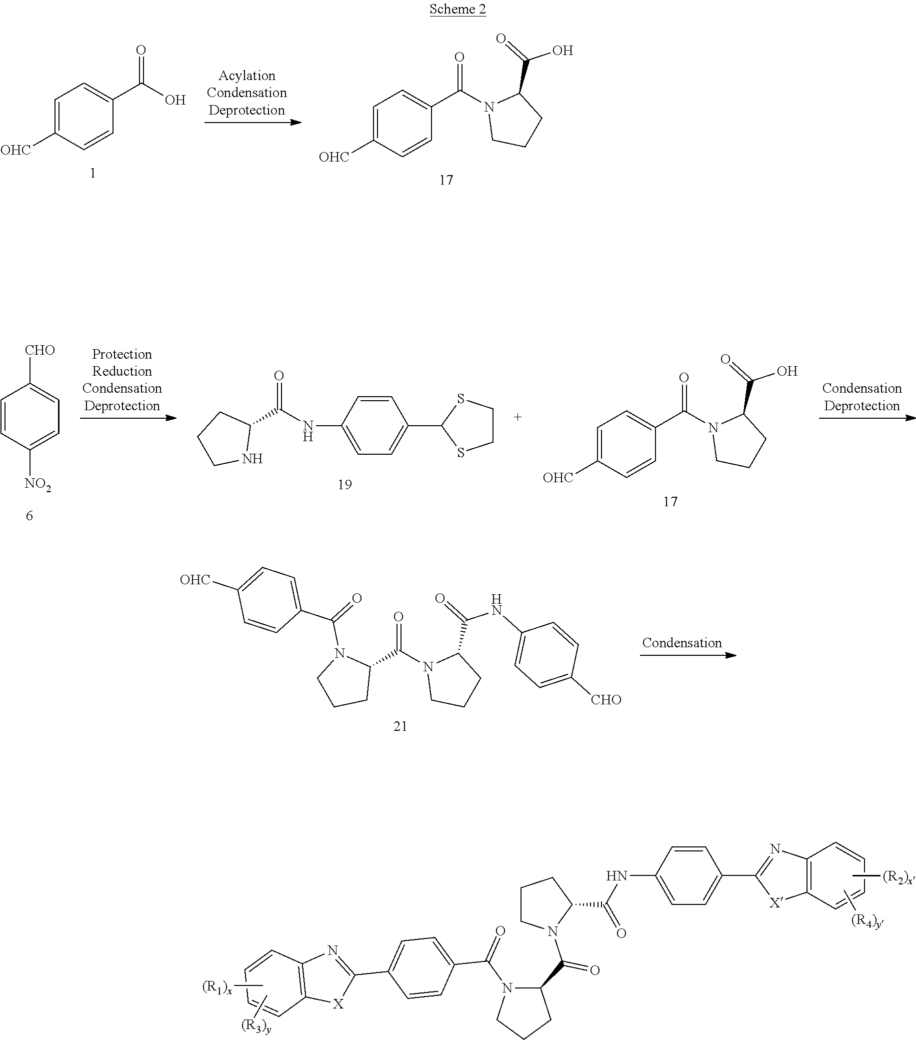

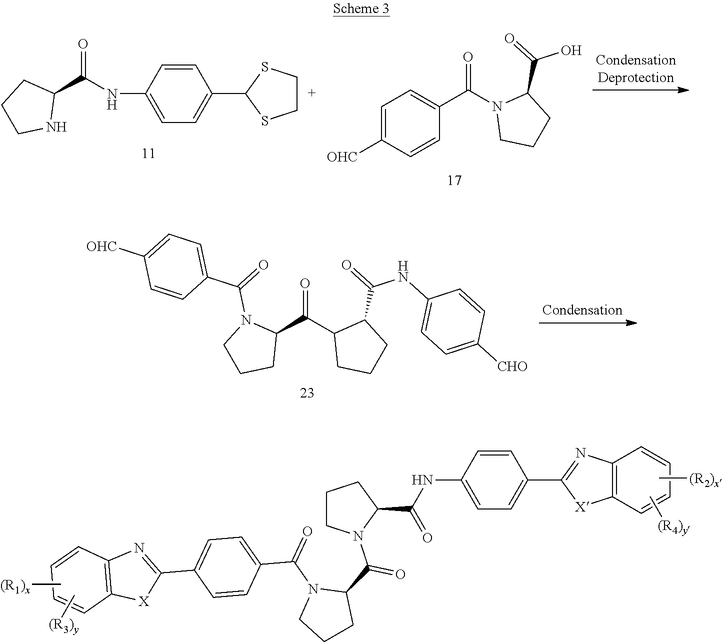

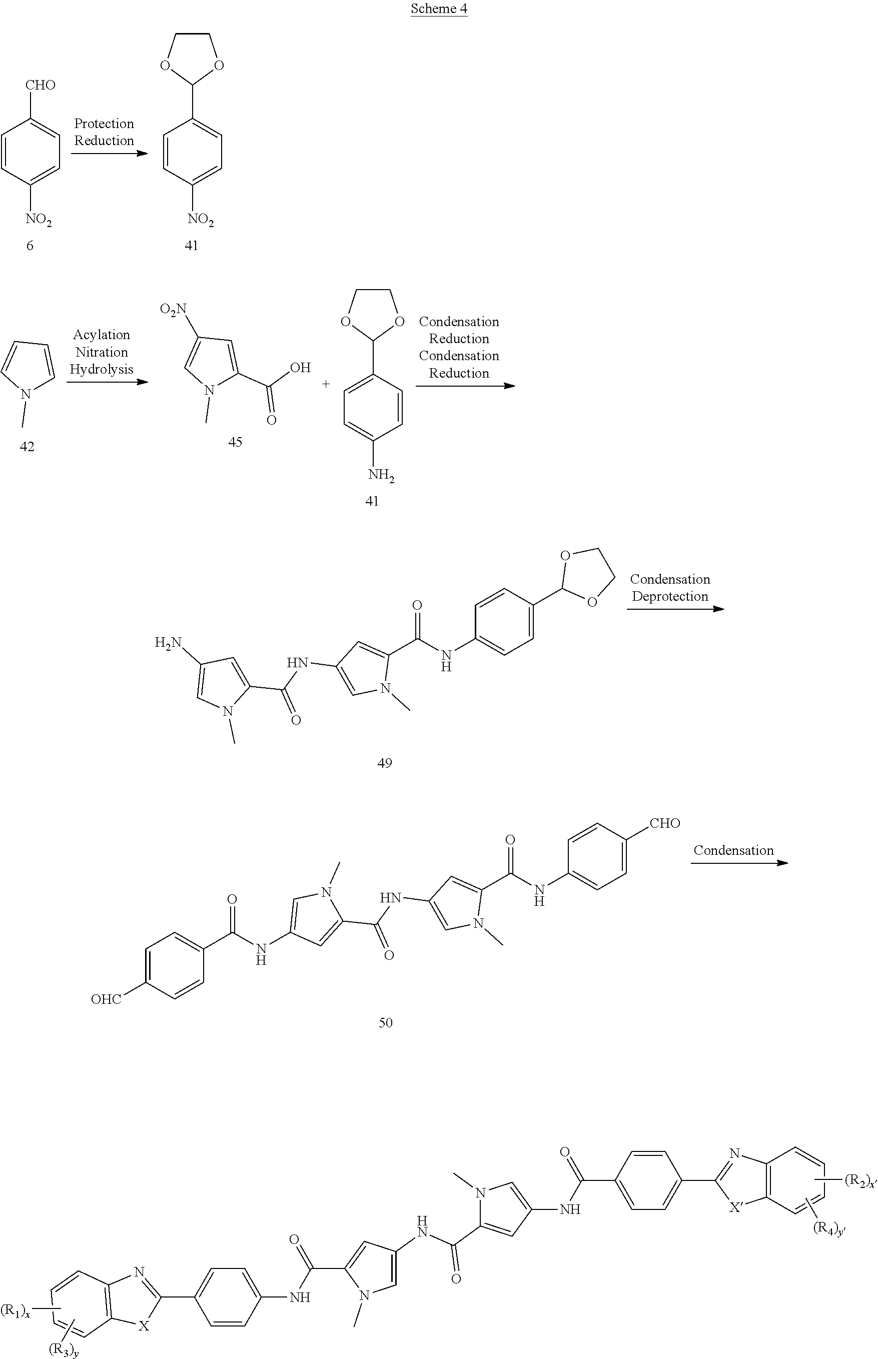

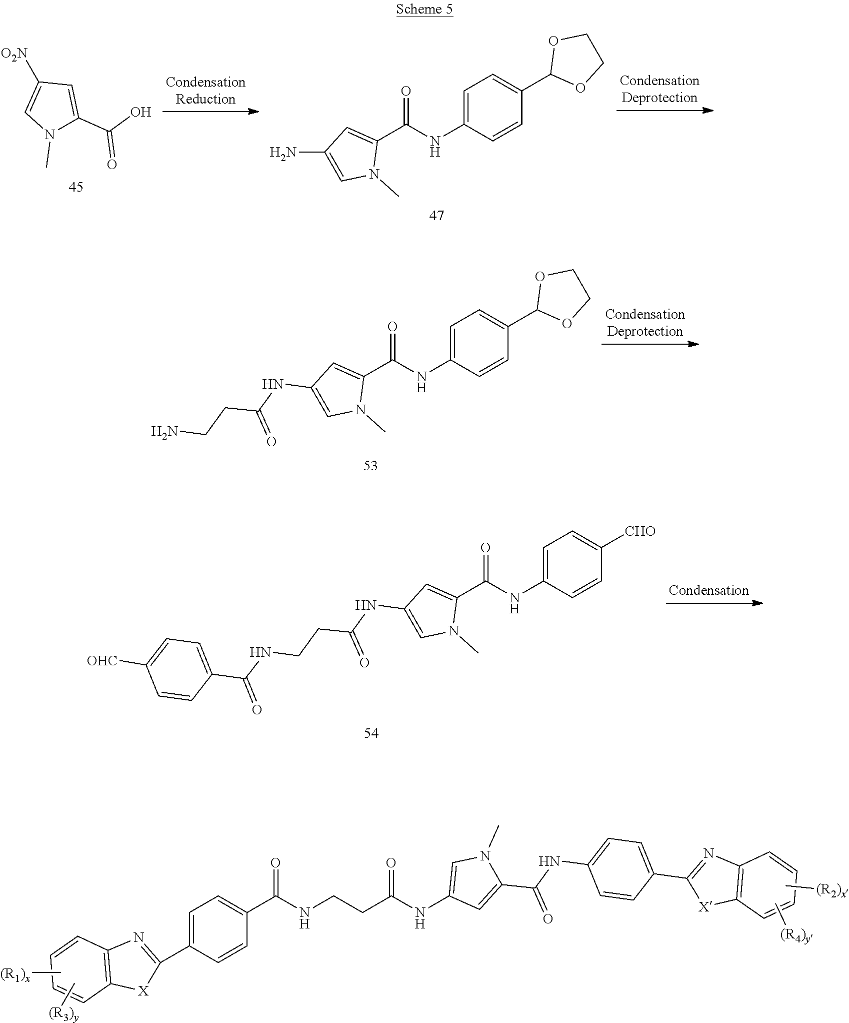

Compounds of formula (1) or any related formula described herein can be synthesized using standard synthetic techniques known to those of ordinary skill in the art. Compounds of the present disclosure can be synthesized using the general synthetic procedures set forth in the schemes provided above and examples provided below.

Where it is desired to obtain a particular enantiomer of a compound, this may be accomplished from a corresponding mixture of enantiomers using any suitable conventional procedure for separating or resolving enantiomers. Thus, for example, diastereomeric derivatives may be produced by reaction of a mixture of enantiomers, e.g. a racemate and an appropriate chiral compound. The diastereomers may then be separated by any convenient means, for example by crystallization and the desired enantiomer recovered. In another resolution process, a racemate may be separated using chiral High-Performance Liquid Chromatography. Alternatively, if desired, a particular enantiomer may be obtained by using an appropriate chiral intermediate in one of the processes described.

### EXAMPLES

### Synthetic Examples

Compounds disclosed herein can be prepared from commercially available starting materials and preparing methods described herein. The following examples serve to illustrate the compounds disclosed herein and the preparation processes thereof. These examples and preparation processes described below should not be considered as limiting the scope of the present disclosure.

The structures of the compounds in the present disclosure were confirmed by ¹H NMR. All of the compounds or intermediates in the synthetic steps were purified by column chromatography, or preparative reverse-phase HPLC unless otherwise specified. The reaction process can be detected by thin layer chromatography, and the commonly used elution systems in the purification stage were petroleum ether/ethyl acetate and dichloromethane/methanol.

### Example S1: Synthesis of (S)-1-((4-(6-carbamimidoyl-1H-benzo[d]imidazol-2 yl)benzoyl)-L-prolyl)-N-(4-(6-carbamimidoyl-1H-benzo[d]imidazol-2 yl)phenyl)pyrrolidine-2-carboxamide (Compound I-1)

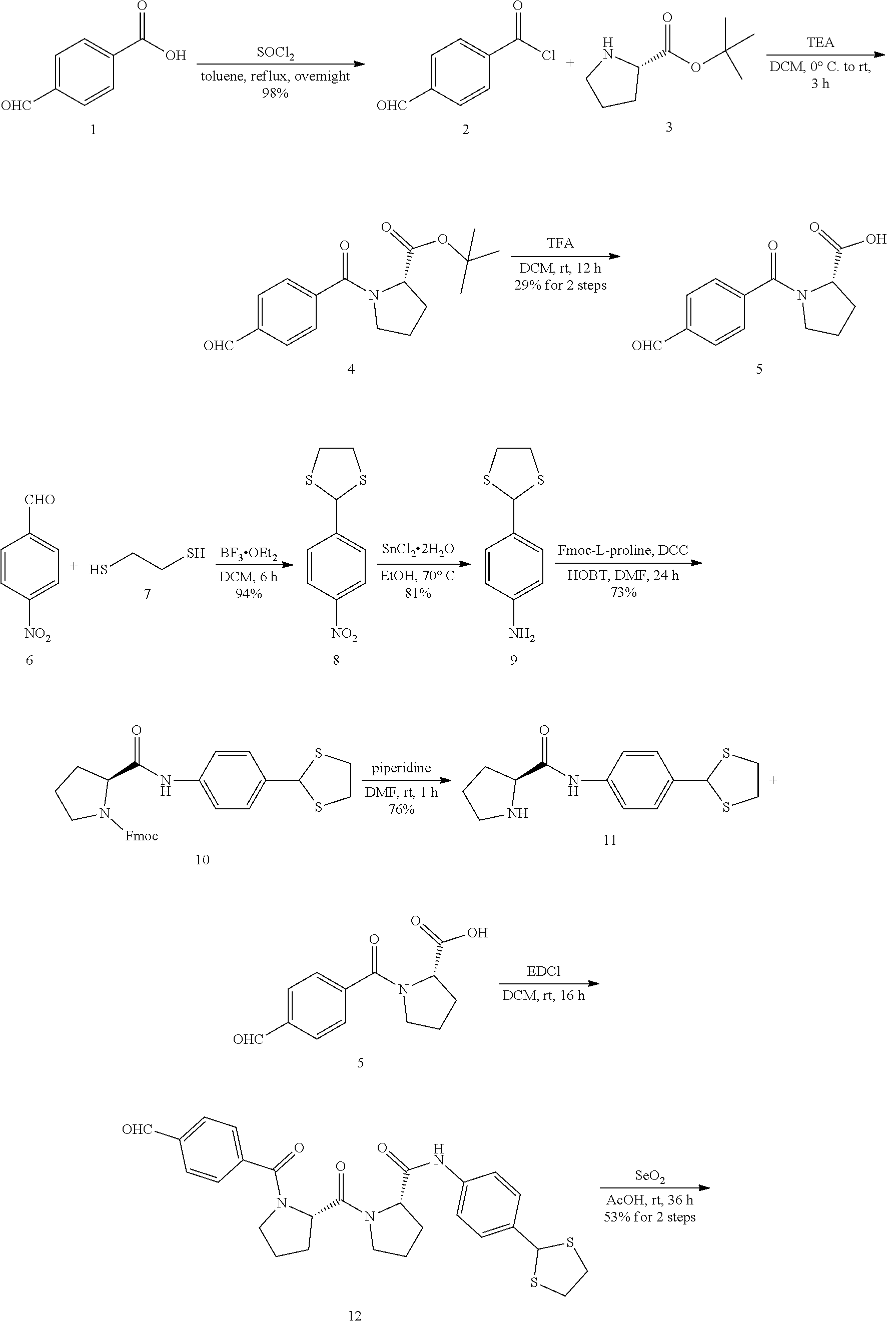

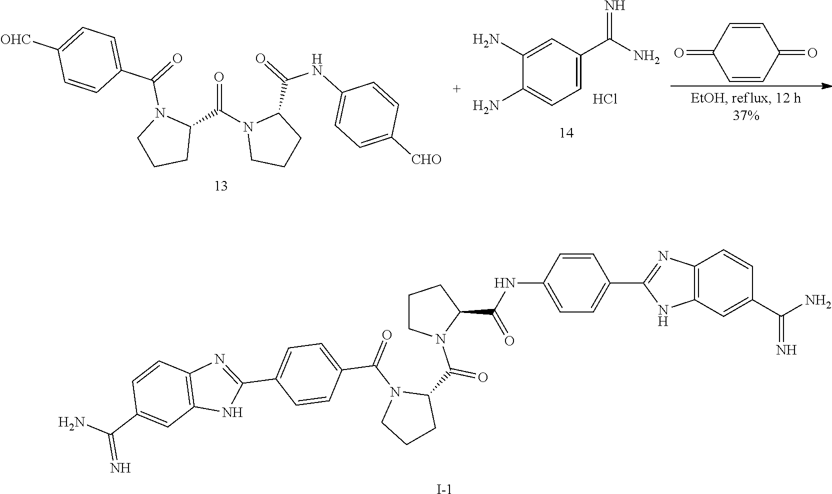

Step 1: Synthesis of 4-formylbenzoyl chloride (compound 2). 4-Formylbenzoic acid 1 (4 g, 26.64 mmol) was suspended in a mixture of toluene (64 mL) and SOCl₂ (8 L), and the mixture was refluxed at 110° C. overnight. The resulting clear solution was allowed to cool to room temperature and concentrated in vacuo. Excess SOCl₂ was removed by coevaporation with toluene and dried under vacuum to give the desired product 2 as a white solid (4.40 g, 98%).

¹H NMR (400 MHz, CDCl₃) δ 10.15 (s, 1H), 8.29 (d, J=8.3 Hz, 2H), 8.03 (d, J=8.6 Hz, 2H).

Step 2: Synthesis of tert-butyl (4-formylbenzoyl)-L-prolinate (compound 4). To a solution of tert-butyl L-prolinate 3 (2.01 g, 11.74 mmol) in DCM (18 mL) and TEA (2 mL) was added a solution of 2 (1.98 g, 11.74 mmol) in DCM (18 mL) at 0° C. slowly. Then the mixture was warmed to room temperature and stirred for another 3 h. The reaction mixture was washed with aqueous HCl (1 M, 3×60 mL). The combined aqueous fractions were extracted with DCM and the combined organic fractions were further washed with saturated aqueous NaHCO₃ solution, dried over anhydrous Na₂SO₄, filtered, and concentrated in vacuo to obtain a light yellow oil 4 (1.87 g), which was used in the next step without further purification.

Step 3: Synthesis of (4-formylbenzoyl)-L-proline (compound 5). A solution of 4 (1.87 g, 6.16 mmol) in DCM (18 mL) and TFA (18 mL) was stirred at room temperature for 12 h. After the reaction was complete, the solvent was removed. The residue was dissolved in saturated aqueous NaHCO₃solution, and washed with EtOAc. The organic fractions were extracted with saturated aqueous NaHCO₃solution. The aqueous fractions were acidified by adding 2 M HCl until pH=2, and then the combined aqueous fractions were extracted with EtOAc. The combined organic layers were washed with water, dried over anhydrous Na₂SO₄, filtered, and concentrated in vacuo. The crude product was purified by silica gel chromatography (2% MeOH in DCM) to give the desired product 5 as a white solid (850 mg, 29% for 2 steps).

¹H NMR (400 MHz, CDCl₃) δ 10.07 (s, 1H), 7.96 (d, J=8.2 Hz, 2H), 7.72 (d, J=8.1 Hz, 2H), 4.78 (dd, J=8.3, 5.0 Hz, 1H), 3.58-3.51 (m, 2H), 3.34 (s, 1H), 2.41-2.35 (m, 1H), 2.31-2.25 (m, 1H), 2.12-2.02 (m, 1H), 2.00-1.90 (m, 1H).

Step 4: Synthesis of 2-(4-nitrophenyl)-1,3-dithiolane (compound 8). To a solution of 4-nitrobenzaldehyde 6 (6.92 g, 45.79 mmol) in DCM (180 mL) was added ethane-1,2-dithiol 7 (20 mL, 0.24 mol), followed by boron trifluoride diethyl etherate (1.2 mL). After stirring at room temperature for 6 h, the solution was washed with 10% NaOH, water, and brine. The resulting bright yellow solution was dried over anhydrous Na₂SO₄, filtered, and concentrated in vacuo to obtain the desired product 8 as a yellow solid (9.78 g, 94%).

¹H NMR (400 MHz, CDCl₃) δ 8.17 (d, J=8.4 Hz, 2H), 7.67 (d, J=8.3 Hz, 2H), 5.65 (s, 1H), 3.56-3.48 (m, 2H), 3.45-3.37 (m, 2H).

Step 5: Synthesis of 4-(1,3-dithiolan-2-yl)aniline (compound 9). A. solution of 8 (5.0 g, 22.00 mmol) and stannous chloride dihydrate (24.82 g, 0.11 mol) in absolute EtOH (44 mL) was heated at 70° C. for 0.5 h. After cooling to room temperature, the orange solution was poured onto ice in a large beaker and then treated with saturated aqueous NaHCO₃solution until the pH reached 7-8. Approximately 200 mL EtOAc was added and the mixture was vacuum filtered through a glass funnel. The filtrate was washed with brine, dried over anhydrous Na₂SO₄, filtered, and concentrated in vacuo to give the desired product 9 as a bright yellow solid (3.52 g, 81%).

¹H NMR (400 MHz, CDCl₃) δ 7.32 (d, J=7.8 Hz, 2H), 6.62 (d, J=7.7 Hz, 2H), 5.61 (s, 1H), 3.69 (s, 2H), 3.53-3.45 (m, 2H), 3.37-3.29 (m, 2H).

Step 6: Synthesis of (9H-fluoren-9-yl)methyl (S)-2-((4-(1,3-dithiolan-2-yl)phenyl)carbamoyl)pyrrolidine-1-carboxylate (compound 10). To a solution of freshly prepared 9 (3.08 g, 15.61 mmol) and Fmoc-L-proline (5.26 g, 15.59 mmol) in DMF (15 mL) was added a solution of HOBT in DMF (1 M, 15 mL) and a solution of DCC in DCM (1 M, 15 mL), and the reaction mixture was stirred at room temperature for 24 h. Then 75 mL EtOAc was added and the mixture was filtered through a glass funnel. After removal of the solvent, the residue was diluted with CHCl₃/i-PrOH (3:1), then washed with water, 0.1 M HCl, saturated aqueous NaHCO₃solution, and brine. The organic layers were dried over anhydrous Na₂SO₄, filtered, and concentrated in vacuo. The crude product was purified by silica gel chromatography (0-0.5% MeOH in DCM) to give the desired product 10 as a light yellow solid (5.89 g, 73%).

¹H NMR (400 MHz, CDCl₃) δ 9.18 (s, 1H), 7.82-7.28 (m, 12H), 5.62 (s, 1H), 4.56-4.40 (m, 3H), 4.26 (s, 1H), 3.57-3.30 (m, 6H), 2.56 (s, 1H), 1.96 (s, 3H).

Step 7: Synthesis of (S)—N-(4-(1,3-dithiolan-2-yl)phenyl)pyrrolidine-2-carboxamide (compound 11). To a solution of 10 (3.16 g, 6.12 mmol) in DMF (24 mL) was added piperidine (6 mL), and the reaction mixture was stirred at room temperature for 1 h. After removal of the solvent, the residue was dissolved in EtOAc and washed by brine. The organic layers were dried over anhydrous Na₂SO₄, filtered, and concentrated in vacuo. The crude product was purified by silica gel chromatography (0-2% MeOH in DCM) to give the desired product 11 as a white solid (1.37 g, 76%).

¹H NMR (400 MHz, CDCl₃) δ 9.75 (s, 1H), 7.55 (d, J=7.6 Hz, 2H), 7.48 (d, J=7.5 Hz, 2H), 5.63 (s, 1H), 3.85 (dd, J=9.0, 5.2 Hz, 1H), 3.53-3.46 (m, 2H), 3.38-3.31 (m, 2H), 3.11-3.05 (m, 1H), 3.00-2.94 (m, 1H), 2.26-2.16 (m, 1H), 2.07-1.99 (m, 1H), 1.79-1.70 (m, 2H).

Step 8: Synthesis of (S)—N-(4-(1,3-dithiolan-2-yl)phenyl)-1-((4-formylbenzoyl)-L-prolyl)pyrrolidine-2-carboxamide (compound 12). To a solution of 11 (880 mg, 2.99 mmol) and 5 (740 mg, 2.99 mmol) in DCM (20 mL) was added EDCI (688 mg, 3.59 mmol), and the reaction mixture was stirred at room temperature for 16 h. Then the solution was concentrated in vacuo to obtain a white solid 12 (900 mg), which was used in the next step without further purification.

Step 9: Synthesis of (S)-1-((4-formylbenzoyl)-L-prolyl)-N-(4-formylphenyl)pyrrolidine-2-carboxamide (compound 13). To a solution of 12 (900 mg, 1.72 mmol) in AcOH (35 mL) was added SeO₂ (954 mg, 8.60 mmol), and the reaction mixture was stirred at room temperature for 36 h. The mixture was filtered, and the filtrate was evaporated under reduced pressure. The residue was dissolved in DCM, washed with saturated aqueous NaHCO₃solution, dried over anhydrous Na₂SO₄, filtered, and concentrated in vacuo. The crude product was purified by silica gel chromatography (pure EtOAc) to give the desired product 13 as a white solid (710.1 mg, 53% for 2 steps).

¹H NMR (400 MHz, CDCl₃) δ 10.07 (d, J=8.0 Hz, 1H), 9.93 (dd, J=26.6, 22.2 Hz, 2H), 8.13 (d, J=8.6 Hz, 0.5H), 7.95 (dd, J=13.0, 8.0 Hz, 2H), 7.85 (d, J=8.7 Hz, 0.5H), 7.79-7.67 (m, 5H), 4.87-4.81 (m, 1.5H), 4.55 (dd, J=17.1, 7.7 Hz, 0.5H), 3.97 (dd, J=16.8, 9.1 Hz, 1H), 3.76-3.62 (m, 2H), 3.57-3.52 (m, 1H), 2.49-1.95 (m, 8H).

Step 10: synthesis of (S)-1-((4-(6-carbamimidoyl-1H-benzo[d]imidazol-2-yl)benzoyl)-L-prolyl)-N-(4-(6-carbamimidoyl-1H-benzo[d]imidazol-2-yl)phenyl)pyrrolidine-2-carboxamide (compound I-1). A solution of 13 (143.4 mg, 0.32 mmol), 3,4-diaminobenzimidamide hydrochloride 14 (120 mg, 0.64 mmol) and p-benzoquinone (70.0 mg, 0.64 mmol) in anhydrous EtOH (13 mL) was heated under reflux for 12 h. The reaction mixture was cooled to room temperature, and stirred in acetone (80 mL) for 0.5 h. The mixture was filtered, washed with dry ether, and dried to give a brown solid. Then the solid was dissolved in a 1:1 mixture of hot MeOH (30 mL) and EtOH (30 mL), filtered, the volume was reduced to 20 mL and acidified with HCl-saturated EtOH (2 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in 1120 with 0.05% HCl) to give the desired product I-1 as a brown solid (101.7 mg, 37%).

¹H NMR (400 MHz, Methanol-d₄) δ 8.27-8.23 (m, 4H), 8.15 (d, J=8.6 Hz, 2H), 7.94 (d, J=8.8 Hz, 4H), 7.90-7.82 (m, 4H), 4.77-4.72 (m, 1H), 4.06-3.99 (m, 1H), 3.86-3.60 (m, 4H), 2.59-2.50 (m, 1H), 2.45-2.36 (m, 1H), 2.24-1.99 (m, 6H).

### Example S2: Synthesis of (R)-1-((4-(6-carbamimidoyl-1H-benzo[d]imidazol-2 yl)benzoyl)-D-prolyl)-N-(4-(6-carbamimidoyl-1H-benzo[d]imidazol-2 yl)phenyl)pyrrolidine-2-carboxamide (Compound 1-2)

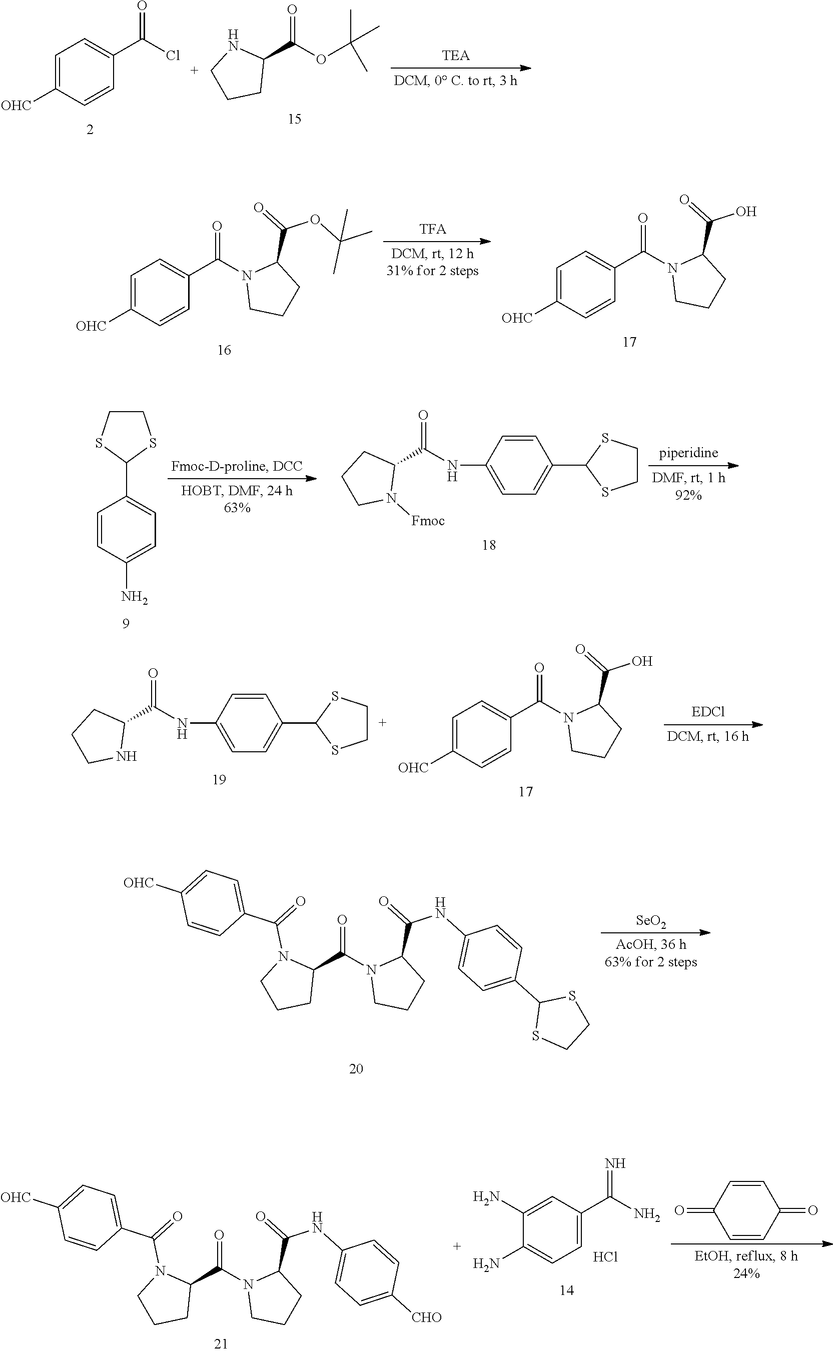

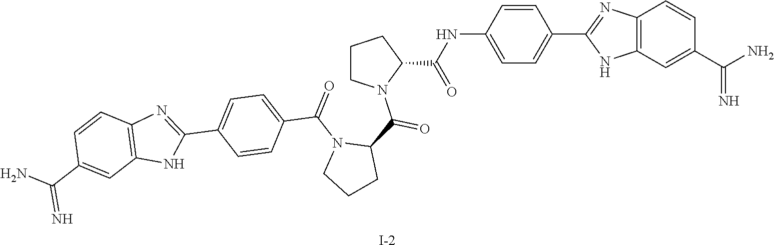

Step 1: Synthesis of tert-butyl (4-formylbenzoyl)-D-prolinate (compound 16). To a solution of tert-butyl D-prolinate 15 (1 g, 5.84 mmol) in DCM (10 mL) and TEA (1 mL) was added a solution of 2 (984 mg, 11.74 mmol) in DCM (10 mL) at 0° C. slowly. Then the mixture was warmed to room temperature and stirred for another 3 h. The reaction mixture was washed with aqueous HCl (1 M., 3×30 mL). The combined aqueous fractions were extracted with DCM and the combined organic fractions were further washed with saturated aqueous NaHCO₃solution, dried over anhydrous Na₂SO₄, filtered, and concentrated in vacuo to obtain a light yellow oil 16 (798.1 mg), which was used in the next step without further purification.

Step 2: Synthesis of (4-formylbenzoyl)-D-proline (compound 17). A solution of 16 (798.1 mg, 2.63 mmol) in DCM (9 mL) and TFA (9 mL) was stirred at room temperature for 12 h. After the reaction was complete, the solvent was removed. The residue was dissolved in saturated aqueous NaHCO₃solution, and washed with EtOAc. The organic fractions were extracted with saturated aqueous NaHCO₃solution. The aqueous fractions were acidified by adding 2 M HO until pH=2, and then was extracted with EtOAc. The combined organic layers were washed with water, dried over anhydrous Na₂SO₄, filtered, and concentrated in vacuo. The crude product was purified by silica gel chromatography (2% MeOH in DCM) to give the desired product 17 as a white solid (444 mg, 31% for 2 steps).

¹H NMR (400 MHz, CDCl₃) δ 10.07 (s, 1H), 8.58 (s, 1H), 7.96 (d, J=7.9 Hz, 2H), 7.72 (d, J=7.6 Hz, 2H), 4.76 (t, 0.7=6.7 Hz, 1H), 3.62-3.50 (m, 2H), 2.32 (dd, J=13.5, 6.7 Hz, 2H), 2.12-2.02 (m, 1H), 2.00-1.90 (m, 1H).

Step 3: Synthesis of (9H-fluoren-9-yl)methyl (R)-2-((4-(1,3-dithiolan-2-yl)phenyl)carbamoyl)pyrrolidine-1-carboxylate (compound 18). To a solution of freshly prepared 9 (1.90 g, 9.63 mmol) and Fmoc-D-proline (3.25 g, 9.63 mmol) in DMF (9.5 mL) was added a solution of HOBT in DMF (1 M, 9.6 mL) and a solution of DCC in DCM (1 M. 9.6 mL), and the reaction mixture was stirred at room temperature for 24 h. Then 50 mL EtOAc was added and the mixture was filtered through a glass funnel. After removal of the solvent, the residue was diluted with CHCl₃/i-PrOH (3:1), then washed with water, 0.1 M HCl, saturated aqueous NaHCO₃solution, and brine. The organic layers were dried over anhydrous Na₂SO₄, filtered, and concentrated in vacuo. The crude product was purified by silica gel chromatography (0-0.5% MeOH in DCM) to give the desired product 18 as a light yellow solid (3.13 g, 63%).

¹H NMR (400 MHz, CDCl₃) δ 9.18 (s, 1H), 7.81-7.30 (m, 12H), 5.62 (s, 1H), 4.56-4.41 (m, 3H), 4.26 (s, 1H), 3.56-3.31 (m, 6H), 2.57 (s, 1H), 1.98 (s, 3H).

Step 4: Synthesis of (R)—N-(4-(1,3-dithiolan-2-yl)phenyl)pyrrolidine-2-carboxamide (compound 19). To a solution of 18 (1.51 g, 2.92 mmol) in DMF (10 mL) was added piperidine (2.6 mL), and the reaction mixture was stirred at room temperature for 1 h. After removal of the solvent, the residue was dissolved in EtOAc and washed by brine. The organic layers were dried over anhydrous Na₂SO₄, filtered, and concentrated in vacuo. The crude product was purified by silica gel chromatography (0-2% MeOH in DCM) to give the desired product 19 as a white solid (790 mg, 92%).

¹H NMR (400 MHz, CDCl₃) δ 9.74 (s, 1H), 7.55 (d, J=8.6 Hz, 2H), 7.48 (d, J=8.6 Hz, 2H), 5.63 (s, 1H), 3.85 (dd, J=9.3, 5.2 Hz, 1H), 3.54-3.46 (m, 2H), 3.38-3.31 (m, 2H), 3.10-3.04 (m, 1H), 3.00-2.94 (m, 1H), 2.25-2.17 (m, 1H), 2.07-1.99 (m, 1H), 1.79-1.71 (m, 2H).

Step 5: Synthesis of (R)—N-(4-(1,3-dithiolan-2-yl)phenyl)-1-((4-formylbenzoyl)-D-prolyl)pyrrolidine-2-carboxamide (compound 20). To a solution of 19 (210 mg, 0.71 mmol) and 17 (176 mg, 0.71 mmol) in DCM (5 mL) was added EDCI (164 mg, 0.85 mmol), and the reaction mixture was stirred at room temperature for 16 h. Then the solution was concentrated in vacuo to obtain a white solid 20 (319.3 mg), which was used in the next step without further purification.

Step 6: Synthesis of (R)-1-((4-formylbenzoyl)-D-prolyl)-N-(4-formylphenyl)pyrrolidine-2-carboxamide (compound 21). To a solution of 20 (319.3 mg, 0.61 mmol) in AcOH (12 mL) was added SeO₂ (338 mg, 3.05 mmol), and the reaction mixture was stirred at room temperature for 36 h. The mixture was filtered, and the filtrate was evaporated under reduced pressure. The residue was dissolved in DCM, washed with saturated aqueous NaHCO₃solution, dried over anhydrous Na₂SO₄, filtered, and concentrated in vacuo. The crude product was purified by silica gel chromatography (pure EtOAc) to give the desired product 21 as a white solid (200.6 mg, 63% for 2 steps).

¹H NMR (400 MHz, CDCl₃) δ 10.07 (d, J=9.4 Hz, 1H), 9.93 (dd, J=32.9, 28.7 Hz, 2H), 8.13 (d, J=8.6 Hz, 0.5H), 7.94 (dd, J=15.5, 8.1 Hz, 2H), 7.85 (d, J=8.7 Hz, 0.5H), 7.79-7.65 (m, 5H), 4.86-4.80 (m, 1.5H), 4.55 (dd, J=17.1, 7.7 Hz, 0.5H), 3.96 (dd, J=16.7, 9.0 Hz, 1H), 3.76-3.63 (m, 2H), 3.57-3.51 (m, 1H), 2.45-1.90 (m, 8H).

Step 7: Synthesis of (R)-1-((4-(6-carbamimidoyl-1H-benzo[d]imidazol-2-yl)benzoyl)-D-prolyl)-N-(4-(6-carbamimidoyl-1H-benzo[d]imidazol-2-yl)phenyl)pyrrolidine-2-carboxamide (Compound 1-2). A solution of 21 (87.6 mg, 0.20 mmol), 3,4-diaminobenzimidamide hydrochloride 14 (73 mg, 0.39 mmol) and p-benzoquinone (42.6 mg, 0.39 mmol) in anhydrous EtOH (8 mL) was heated under reflux for 8 h. The reaction mixture was cooled to room temperature, and stirred in acetone (50 mL) for 0.5 h. The mixture was filtered, washed with dry ether, and dried to give a brown solid. Then the solid was dissolved in a 1:1 mixture of hot MeOH (18 mL) and EtOH (18 mL), filtered, the volume was reduced to 12 mL and acidified with HCl-saturated EtOH (1.2 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in 1120 with 0.05% HCl) to give the desired product 1-2 as a brown solid (40.4 mg, 24%).

¹H NMR (400 MHz, Methanol-d₄) δ 8.27-8.23 (m, 4H), 8.16 (d, J=8.8 Hz, 2H), 7.97-7.93 (m, 4H), 7.91-7.82 (m, 4H), 4.75-4.70 (m, 1H), 4.06-3.98 (m, 1H), 3.87-3.59 (m, 4H), 2.58-2.50 (m, 1H), 2.44-2.36 (m, 1H), 2.26-1.95 (m, 6H).

### Example S3: Synthesis of(S)-1-((4-(6-carbamimidoyl-1H-benzo(d/imidazol-2 yl)benzoyl)-D-prolyl)-N-(4-(6-carbamimidoyl-1H-benzo[d]imidazol-2 yl)phenyl)pyrrolidine-2-carboxamide (Compound 1-3)

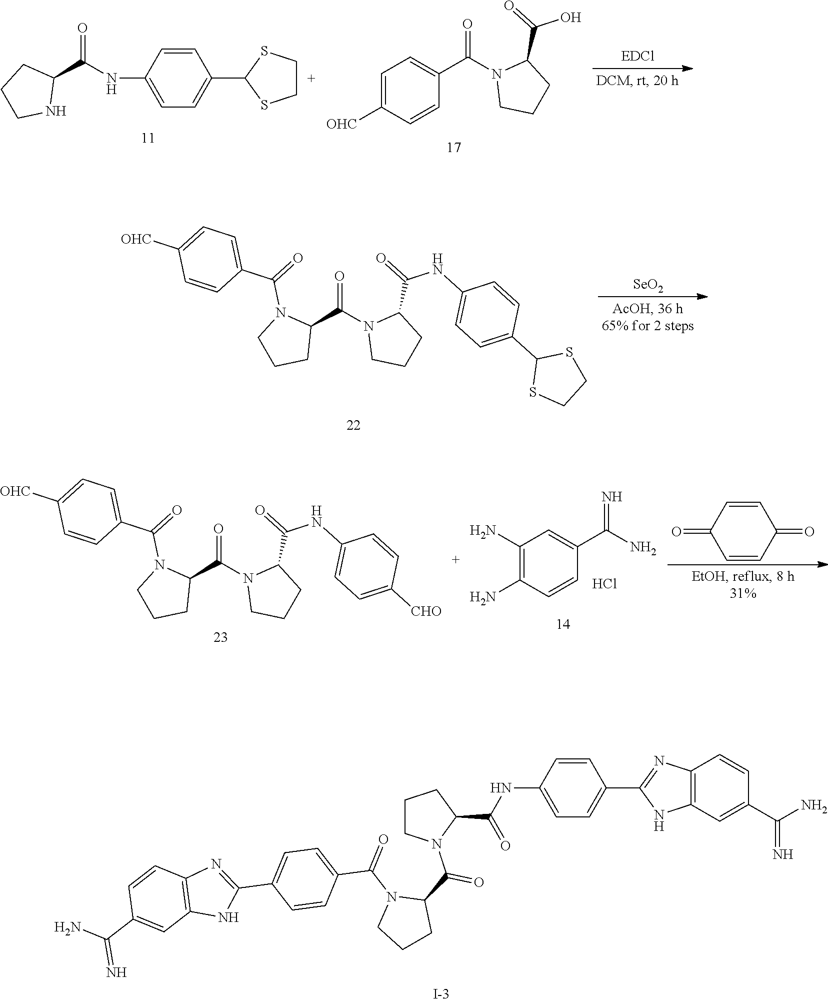

Step 1: Synthesis of (S)—N-(4-(1,3-dithiolan-2-yl)phenyl)-1-((4-formylbenzoyl)-D-prolyl)pyrrolidine-2-carboxamide (compound 22). To a solution of 11 (213 mg, 0.72 mmol) and 17 (179 mg, 0.72 mmol) in DCM (5 mL) was added EDCI (166 mg, 0.86 mmol), and the reaction mixture was stirred at room temperature for 20 h. Then the solution was concentrated in vacuo to obtain a white solid 22 (255 mg), which was used in the next step without further purification.

Step 2: Synthesis of (S)-1-((4-formylbenzoyl)-D-prolyl)-N-(4-formylphenyl)pyrrolidine-2-carboxamide (compound 23). To a solution of 22 (255 mg, 0.49 mmol) in AcOH (10 mL) was added SeO₂ (270 mg, 2.43 mmol), and the reaction mixture was stirred at room temperature for 36 h. The mixture was filtered, and the filtrate was evaporated under reduced pressure. The residue was dissolved in DCM, washed with saturated aqueous NaHCO₃solution, dried over anhydrous Na₂SO₄, filtered, and concentrated in vacuo. The crude product was purified by silica gel chromatography (pure EtOAc) to give the desired product 23 as a white solid (210.5 mg, 65% for 2 steps).

¹H NMR (400 MHz, CDCl₃) δ 10.08 (s, 1H), 9.84 (s, 1H), 9.13 (s, 1H), 7.95 (d, J=7.8 Hz, 4H), 7.69 (dd, J=10.5, 8.4 Hz, 4H), 4.82-4.76 (m, 2H), 4.25-4.19 (m, 1H), 3.73-3.59 (m, 3H), 2.51-2.46 (m, 1H), 2.32-2.10 (m, 6H), 2.01-1.94 (m, 1H).

Step 3: Synthesis of (S)-1-((4-(6-carbamimidoyl-1H-benzo[d]imidazol-2-yl)benzoyl)-D-prolyl)-N-(4-(6-carbamimidoyl-1H-benzo[d]imidazol-2-yl)phenyl)pyrrolidine-2-carboxamide (compound I-3). A solution of 23 (75 mg, 0.17 mmol), 3,4-diaminobenzimidamide hydrochloride 14 (62.5 mg, 0.33 mmol) and p-benzoquinone (36.5 mg, 0.33 mmol) in anhydrous EtOH (8 mL) was heated under reflux for 8 h. The reaction mixture was cooled to room temperature, and stirred in acetone (50 mL) for 0.5 h. The mixture was filtered, washed with dry ether, and dried to give a brown solid. Then the solid was dissolved in a 1:1 mixture of hot MeOH (18 mL) and EtOH (18 mL), filtered, the volume was reduced to 12 mL and acidified with HCl-saturated EtOH (1.2 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in 1120 with 0.05% HCl) to give the desired product 1-3 as a brown solid (44.6 mg, 31%).

¹H NMR (400 MHz, Methanol-d₄) δ 8.41-8.29 (m, 4H), 8.27-8.24 (m, 2H), 8.11 (s, 4H), 8.01-7.93 (m, 4H), 5.00 (t, J=7.1 Hz, 1H), 4.73-4.66 (m, 1H), 4.25-4.18 (m, 1H), 3.88-3.81 (m, 1H), 3.78-3.68 (m, 2H), 2.53-2.45 (m, 1H), 2.42-2.34 (m, 1H), 2.31-2.24 (m, 1H), 2.20-2.00 (m, 5H).

### Example S4: Synthesis of 2-(4-((S)-1-((4-(6-carbamoyl-1H-benzo[d]imidazol-2 yl)benzoyl)-L-prolyl)pyrrolidine-2-carboxamido)phenyl)-1H-benzo[d]imidazole-6-carboxamide (Compound 1-4)

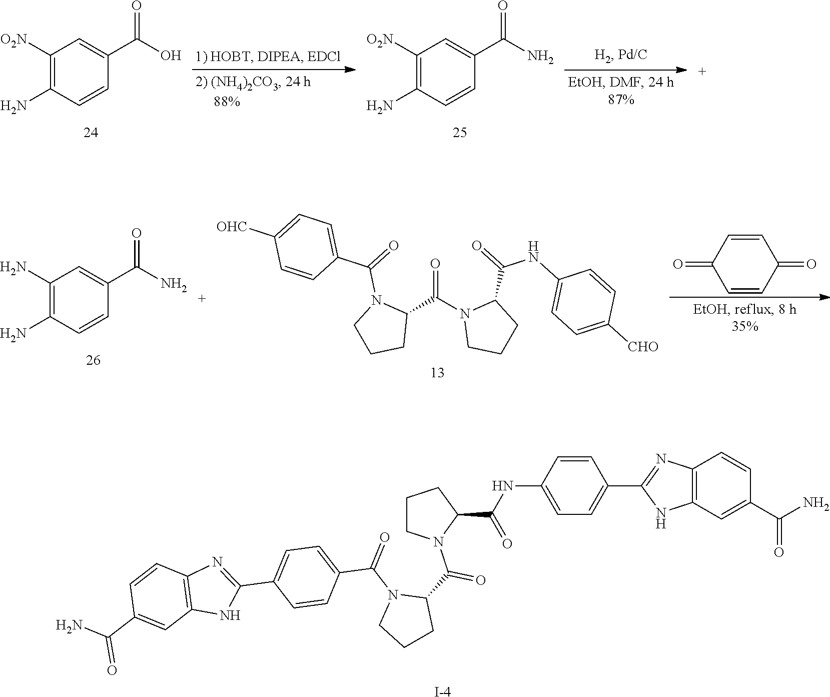

Step 1: Synthesis of 4-amino-3-nitrobenzamide (compound 25). To a stirred suspension of 4-amino-3-nitrobenzoic acid 24 (1 g, 5.49 mmol), HOBT (816 mg, 6.04 mmol) and EDCI (1.16 g, 6.05 mmol) in THE (50 ml) was added DIPEA (1 mL, 6.06 mmol), and the reaction mixture was stirred at room temperature for 10 min. (NH₄)₂CO₃ (1.58 g, 16.44 mmol) was then added in one portion, and the resulting suspension was stirred for an additional 24 h. The reaction mixture was concentrated in vacuo, followed by addition of a 1:1 mixture of NaHCO₃/H₂O (40 mL), and the stirring was continued for 2 h. The suspension was filtered, and solid was dried under vacuum (40° C., 24 h) to afford the desired product 25 as a brown solid (878.7 mg, 88%).

¹H NMR (400 MHz, DMSO-d₆) δ 8.57 (s, 1H), 7.94 (s, 1H), 7.86 (d, J=8.8 Hz, 1H), 7.76 (s, 2H), 7.25 (s, 1H), 7.01 (d, J=8.9 Hz, 1H).

Step 2: Synthesis of 3,4-diaminobenzamide (compound 26). To a solution of 25 (400 mg, 2.21 mmol) in DMF (2 mL) and EtOH (3 mL) was added Pd/C (78 mg, 10%). The flask was then evacuated, flushed three times with H₂, filled with 1-12, and stirred at room temperature for 24 h. The reaction mixture was filtered through a pad of celite, and washed with EtOH. The filtrate was concentrated under reduced pressure to give the crude product, which purified by silica gel chromatography (0.5% MeOH in DCM) to give the desired product 26 as a brown solid (289.2 mg, 87%).

¹H NMR (400 MHz, DMSO-d₆) δ 7.39 (s, 1H), 7.05 (s, 1H), 6.97 (d, J=8.0 Hz, 1H), 6.71 (s, 1H), 6.45 (d, J=8.0 Hz, 1H), 4.94 (s, 2H), 4.50 (s, 2H).

Step 3: Synthesis of 2-(4-((S)-1-((4-(6-carbamoyl-1H-benzo[d]imidazol-2-yl)benzoyl)-L-prolyl)pyrrolidine-2-carboxamido)phenyl)-1H-benzo[d]imidazole-6-carboxamide (compound I-4). A solution of 13 (99.8 mg, 0.22 mmol), 3,4-diaminobenzamide 26 (67.7 mg, 0.45 mmol) and p-benzoquinone (48.6 mg, 0.45 mmol) in anhydrous EtOH (9 mL) was heated under reflux for 8 h. The reaction mixture was cooled to room temperature, and concentrated in vacuo to give a brown solid. Then the solid was dissolved in a 1:1 mixture of hot MeOH (20 mL) and EtOH (20 mL), filtered, the volume was reduced to 13.5 mL and acidified with HCl-saturated EtOH (3 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-4 as a dark green solid (67 mg, 35%).

¹H NMR (500 MHz, DMSO-d₆) δ 10.82 (s. 1H), 10.72 (s, 0.5H), 8.52-8.21 (m, 14H), 8.08-7.76 (m, 12H), 7.66 (d, J=8.0 Hz, 1H), 7.52 (d, J=14.2 Hz, 3H), 4.83 (dd, J=8.3, 4.5 Hz, 1H), 4.75 (dd, J=8.4, 3.5 Hz, 0.5H), 4.60 (dd, J=8.3, 4.8 Hz, 1H), 4.17 (dd, J=8.1, 4.5 Hz, 0.5H), 3.87-3.79 (m, 1H), 3.71-3.48 (m, 4H), 3.40-3.32 (m, 0.5H), 3.08-3.00 (m, 0.5H), 2.39-2.33 (m, 1H), 2.30-2.21 (m, 1H), 2.10-1.76 (m, 9H), 1.74-1.63 (m, 1H).

### Example 55: Synthesis of (S)-1-((4-(1,7-dihydroimidazo[4, 5-f]indazol-6 yl)benzoyl)-L-prolyl)-N-(4-(1,7-dihydroimidazo[4,5-f]indazol-6 yl)phenyl)pyrrolidine-2-carboxamide (Compound 1-5)

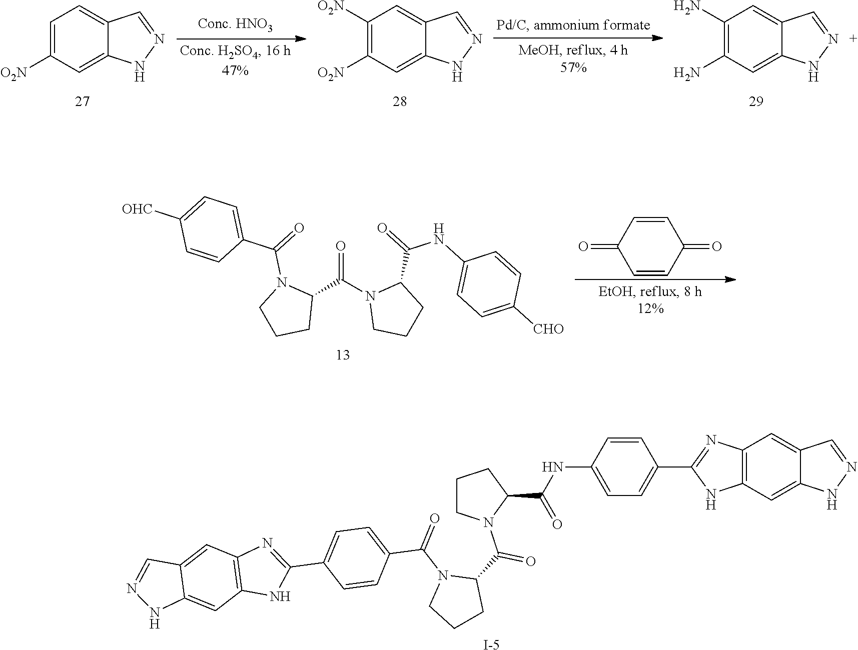

Step 1: Synthesis of 5,6-dinitro-1H-indazole (compound 28). A mixture of 6-nitro-1H-indazole 27 (1 g, 6.13 mmol) in conc. H₂SO₄ (14 mL) was cooled to 0° C. and slowly added into a stirred solution of conc. HNO₃ (0.42 mL) in conc. H₂SO₄ (6 mL) at 0° C. The reaction mixture was stirred at room temperature for 16 h and then was poured onto ice. The solid was filtered off, washed with water, and dissolved in CHCl₃/i-PrOH (3:1). The mixture was then washed with brine, saturated aqueous NaHCO₃ solution, dried over anhydrous Na₂SO₄, filtered, and concentrated in vacuo to give the desired product 28 as a yellow solid (595 mg, 47%).

¹H NMR (400 MHz, DMSO-d₆) δ 14.35 (s, 1H), 8.85 (s, 1H), 8.54 (s, 1H), 8.45 (s, 1H).

Step 2: Synthesis of 1H-indazole-5,6-diamine (compound 29). To a mixture of 28 (300 mg, 1.44 mmol) and Pd/C (30 mg, 10%) in MeOH (9 mL) was added ammonium formate (900 mg, 14.27 mmol), and the mixture was refluxed for 4 h. Then the catalyst was removed by filtration through a celite pad, and was washed with MeOH. The filtrate was concentrated under reduced pressure to give the crude product, which was purified by silica gel chromatography (2-10% MeOH in DCM) to give the desired product 29 as a brown solid (121.4 mg, 57%).

¹H NMR (400 MHz, DMSO-d₆) δ 12.02 (s, 1H), 7.52 (s, 1H), 6.71 (s, 1H), 6.56 (s, 1H), 4.80 (s, 2H), 4.29 (s, 2H).

Step 3: Synthesis of (S)-1-((4-(1,7-dihydroimidazo[4,5-f]indazol-6-yl)benzoyl)-L-prolyl)-N-(4-(1,7-dihydroimidazo[4,5-f]indazol-6-yl)phenyl)pyrrolidine-2-carboxamide (compound I-5). A solution of 13 (77.4 mg, 0.17 mmol), 1H-indazole-5,6-diamine 29 (51.2 mg, 0.34 mmol) and p-benzoquinone (37.7 mg, 0.34 mmol) in anhydrous EtOH (7 mL) was heated under reflux for 8 h. The reaction mixture was cooled to room temperature, and concentrated in vacuo to give a brown solid. Then the solid was dissolved in a 1:1 mixture of hot MeOH (15 mL) and EtOH (15 mL), filtered, the volume was reduced to 10.5 mL and acidified with HCl-saturated EtOH (2.1 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-5 as a brown solid (16.8 mg, 12%).

¹H NMR (600 MHz, DMSO-d₆) δ 10.85 (s, 1H), 10.76 (s, 0.5H), 8.55-8.08 (m, 16H), 7.97 (d, J=8.6 Hz, 2H), 7.93-7.80 (m, 8H), 7.70 (d, J=8.1 Hz, 1H). 4.84 (dd, J=8.3, 4.5 Hz, 1H), 4.77 (dd, J=8.0, 3.7 Hz, 0.5H), 4.59 (dd, J=8.4, 4.7 Hz, 1H), 4.16 (dd, J=8.2, 4.3 Hz, 0.5H), 3.86-3.81 (m, 1H), 3.70-3.57 (m, 4H), 3.40-3.36 (m, 0.5H), 3.10-3.05 (m, 0.5H), 2.40-2.34 (m, 1H), 2.30-2.23 (m, 1H), 2.11-2.05 (m, 1H), 2.04-1.99 (m, 1H), 1.98-1.84 (m, 7H), 1.74-1.66 (m, 1H).

### Example S6: Synthesis of (S)-1-((4-(6-carbamimidoyl-5-methyl-1H-benzo[d]imidazol-2-yl)benzoyl)-L prolyl)-N-(4-(6-carbamimidoyl-5-methyl-1H-benzo[d]imidazol-2-yl)phenyl)pyrrolidine-2-carboxamide (Compound 1-6)

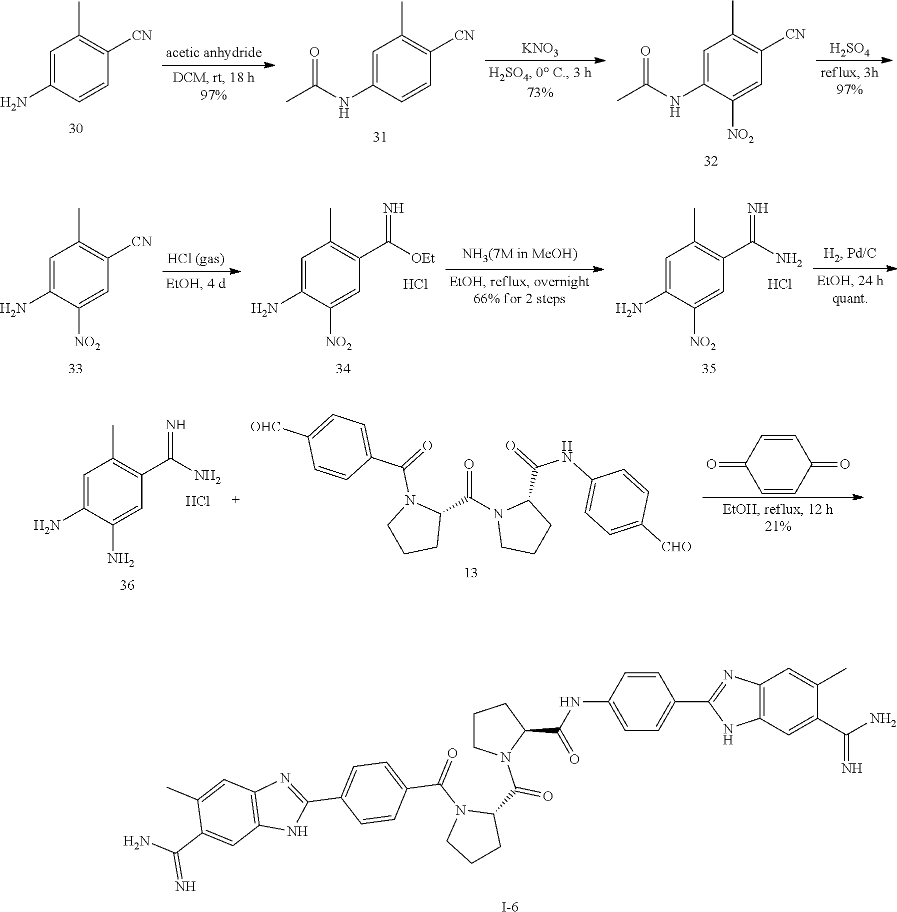

Step 1: Synthesis of N-(4-cyano-3-methylphenyl)acetamide (compound 31). To a solution of 4-amino-2-methylbenzonitrile 30 (4.68 g, 35.41 mmol) in DCM (145 mL) was added Ac₂O (4.32 mL. 42.49 mmol) dropwise, and the reaction mixture was stirred at room temperature for 18 h. After completion of the reaction, the solvent was removed under reduced pressure to give the crude product, which was purified by silica gel chromatography (pure DCM) to give the desired product 31 as a white solid (5.98 g, 97%). ¹H NMR (400 MHz, CDCl₃) δ 7.56 (s, 1H), 7.54 (d, J=8.5 Hz, 1H), 7.40 (d, J=8.4 Hz, 1H), 7.32 (s, 1H), 2.52 (s, 3H), 2.21 (s, 3H).

Step 2: Synthesis of N-(4-cyano-5-methyl-2-nitrophenyl)acetamide (compound 32). To a solution of KNO₃ (3 g, 29.67 mmol) in conc. H₂SO₄ (50 mL) at 0° C. was added 31 (2.6 g, 14.92 mmol). The reaction mixture was stirred for 3 h at 0° C. and then was poured onto ice. The resulting precipitate was recrystallized from MeOH to give the desired product 32 as a yellow solid (2.39 g, 73%).

¹H NMR (400 MHz, CDCl₃) δ 10.53 (s, 1H), 8.86 (s, 1H), 8.48 (s, 1H), 2.61 (s, 3H), 2.32 (s, 3H).

Step 3: Synthesis of 4-amino-2-methyl-5-nitrobenzonitrile (compound 33). A mixture of 32 (1.17 g, 5.34 mmol) in H₂SO₄ (70 mL, 10%) was heated under reflux for 3 h. After cooling to room temperature, the mixture was extracted with CHCl₃/i-PrOH (3:1), and the combined organic layers were dried over anhydrous Na₂SO₄, filtered, and concentrated in vacuo. The crude product was purified by silica gel chromatography (pure DCM) to give the desired product 33 as a yellow solid (920 mg, 97%).

¹H NMR (400 MHz, DMSO-d₆) δ 8.38 (s, 1H), 7.98 (s, 2H), 6.93 (s, 1H), 2.35 (s, 3H).

Step 4: Synthesis of ethyl 4-amino-2-methyl-5-nitrobenzimidate hydrochloride (compound 34). Dry HCl gas was passed through a stirred suspension of 33 (354 mg, 2.00 mmol) in EtOH (20 mL) cooled in an ice-salt bath until the reaction mixture was saturated with HCl, and the mixture was stirred at room temperature for 4 d. The reaction mixture was then concentrated under reduced pressure to yield a yellow mixture of 33 and 34 (482.9 mg), which was used in the next step without further purification.

Step 5: Synthesis of 4-amino-2-methyl-5-nitrobenzimidamide hydrochloride (compound 35). To a mixture of 33 and 34 (482.9 mg, 1.86 mmol) in EtOH (4 mL) was added NH₃ (7 M in MeOH, 6 mL), and the reaction mixture was refluxed overnight. Then the mixture was concentrated in vacuo to give the crude product, which was purified by silica gel chromatography (10-20% MeOH in DCM) to remove unreacted 33 and obtain an orange residue containing 35 (306.3 mg, 66% for 2 steps).

¹H NMR (400 MHz, DMSO-d₆) δ 9.18 (s, 2H), 9.04 (s, 1H), 8.14 (s, 1H), 7.85 (s, 2H), 6.94 (s, 1H), 2.31 (s, 3H).

Step 6: Synthesis of 4,5-diamino-2-methylbenzimidamide hydrochloride (compound 36). To a solution of 35 (304 mg, 1.32 mmol) in EtOH (30 mL) was added Pd/C (60.8 mg, 10%). The flask was then evacuated, flushed three times with H₂, filled with H₂, and stirred at room temperature for 24 h. The reaction mixture was filtered through a pad of celite, and washed with MeOH. The filtrate was concentrated under reduced pressure to obtain a yellow solid 36 (280 mg, quant.).

¹H NMR (400 MHz, Methanol-d₄) δ 6.81 (s, 1H), 6.60 (s, 1H), 2.29 (s, 3H).

Step 7: Synthesis of (S)-1-((4-(6-carbamimidoyl-5-methyl-1H-benzo[d]imidazol-2-yl)benzoyl)-L-prolyl)-N-(4-(6-carbamimidoyl-5-methyl-1H-benzo[d]imidazol-2-yl)phenyl)pyrrolidine-2-carboxamide (compound I-6). A solution of 13 (61.4 mg, 0.14 mmol), 4,5-diamino-2-methylbenzimidamide hydrochloride 36 (55.1 mg, 0.27 mmol) and p-benzoquinone (29.9 mg, 0.27 mmol) in anhydrous EtOH (6 mL) was heated under reflux for 12 h. The reaction mixture was cooled to room temperature, and stirred in acetone (50 mL) for 0.5 h. The mixture was filtered, washed with dry ether, and dried to give a brown solid. Then the solid was dissolved in a 1:1 mixture of hot MeOH (13 mL) and EtOH (13 mL), filtered, the volume was reduced to 9 mL and acidified with HCl-saturated EtOH (0.9 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-6 as a brown solid (26.0 mg, 21%).

¹H NMR (400 MHz, Methanol-d₄) δ 9.53 (d, J=8.0 Hz, 1H), 9.21 (d, J=8.5 Hz, 1H), 8.30-8.27 (m, 2H), 8.20-8.16 (m, 2H), 8.07-8.00 (m, 3H), 7.97-7.84 (m, 3H), 4.97 (dd, J=8.2, 5.6 Hz, 1H), 4.69 (dd, J=8.2, 4.8 Hz, 1H), 4.05-3.98 (m, 1H), 3.86-3.77 (m, III), 3.71-3.56 (m, 2H), 2.67 (d, J=4.6 Hz, 6H), 2.56-2.47 (m, 1H), 2.44-2.36 (m, 1H), 2.28-1.94 (m, 6H).

### Example S7: Synthesis of Hexyl ((2-(4-((S)-1-((4-(6-(N-((hexyloxy)carbonyl)carbamimidoyl)-1H-benzo[d]imidazol-2 yl)benzoyl)-L prolyl)pyrrolidine-2-carboxamido)phenyl)-1H-benzo[d]imidazol-6 yl)(imino)methyl)carbamate (Compound 1-7)

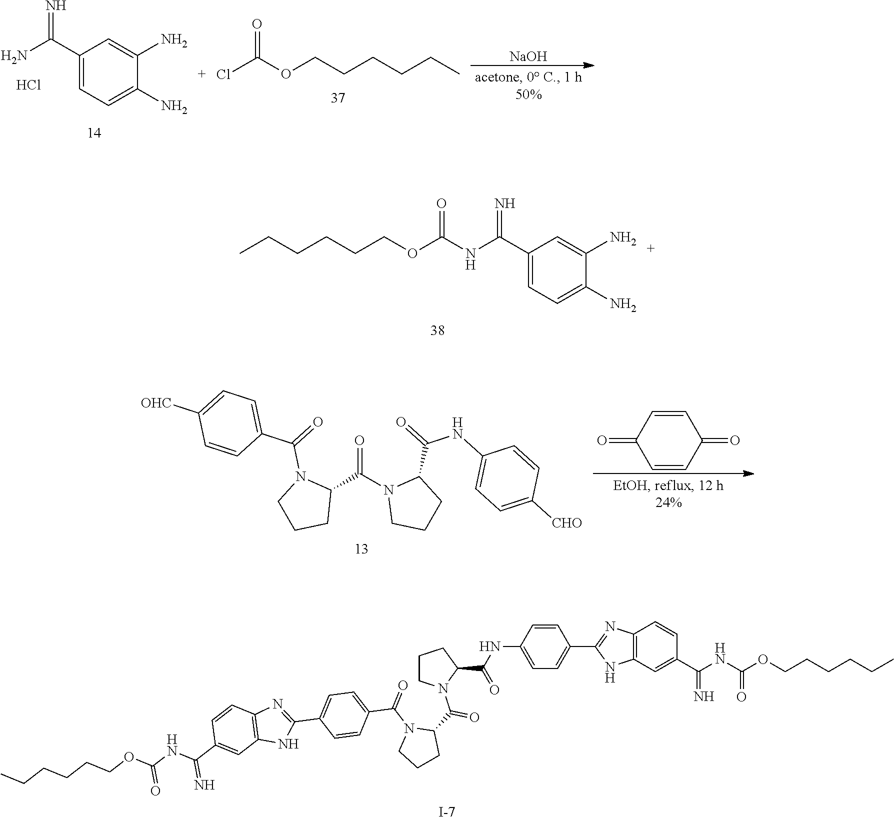

Step 1: Synthesis of hexyl ((3,4-diaminophenyl)(imino)methyl)carbamate (compound 38). A solution of 14 (1.25 g, 6.70 mmol) in acetone (5 mL) was cooled to 0° C. under ice/water bath, followed by slow addition of NaOH solution (5 mL, 16 wt %) and 37 (1.1 mL, 6.70 mmol), and the reaction mixture was stirred at 0° C. for a further 1 h. After cooling to room temperature, the mixture was concentrated under reduced pressure, diluted with CHCl₃/i-PrOH (3:1), and then washed with water. The organic layer was separated, dried over anhydrous Na₂SO₄, filtered, and concentrated in vacuo. The crude product was purified by silica gel chromatography (2% MeOH in DCM) to give the desired product 38 as a light yellow solid (926.3 mg, 50%).

¹H NMR (400 MHz, Methanol-d₄) δ 7.23 (d, J=2.1 Hz, 1H), 7.19 (dd, J=8.2, 2.1 Hz, 1H), 6.69 (d, J=8.2 Hz, 1H), 4.10 (t, J=6.7 Hz, 2H), 1.72-1.65 (m, 2H), 1.47-1.39 (m, 2H), 1.37-1.32 (m, 4H), 0.92 (t, J=6.9 Hz, 3H).

Step 2: Synthesis of hexyl ((2-(4-((S)-1-((4-(6-(N-((hexyloxy)carbonyl)carbamimidoyl)-1H-benzo[d]imidazol-2-yl)benzoyl)-L-prolyl)pyrrolidine-2-carboxamido)phenyl)-1 I1-benzo[d]imidazol-6-yl)(imino)methyl)carbamate (compound I-7). A solution of 13 (37.8 mg, 0.08 mmol), hexyl ((3,4-diaminophenyl)(imino)methyl)carbamate 38 (47.0 mg, 0.16 mmol) and p-benzoquinone (18.4 mg, 0.16 mmol) in anhydrous EtOH (10 mL) was heated under reflux for 12 h. The reaction mixture was cooled to room temperature, and concentrated in vacuo to give a brown solid. Then the solid was dissolved in a 1:1 mixture of hot MeOH (7.5 mL) and EtOH (7.5 mL), filtered, the volume was reduced to 5 mL and acidified with HCl-saturated EtOH (1 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-7 as a brown solid (19.3 mg, 24%).

¹H NMR (400 MHz, Methanol-d₄) δ 8.29-8.13 (m, 6H), 7.98-7.76 (m, 8H), 4.99-4.94 (m, 1H), 4.68 (dd. J=8.3, 4.7 Hz, 1H), 4.41 (td, J=6.7, 2.5 Hz, 4H), 4.07-3.98 (m, 1H), 3.86-3.77 (m, 1H), 3.74-3.59 (m, 2H), 2.55-2.33 (m, 2H), 2.27-1.93 (m, 6H), 1.81 (p, J=6.8 Hz, 411), 1.47 (p, J=6.8 Hz, 4H), 1.38 (h, J=3.5 Hz, 8H), 0.96-0.91 (t, J=6.9 Hz, 6H).

### Example S8: Synthesis of 4-(4-(6-carbamimidoyl-1H-benzo[d]imidazol-2-yl)benzamido)-N-(5-((4-(6-carbamimidoyl-1H-benzo[d]imidazol-2 yl)phenyl)carbamoyl)-1-methyl-1H-pyrrol-3 yl)-1-methyl-1H-pyrrole-2-carboxamide (Compound 1-8)

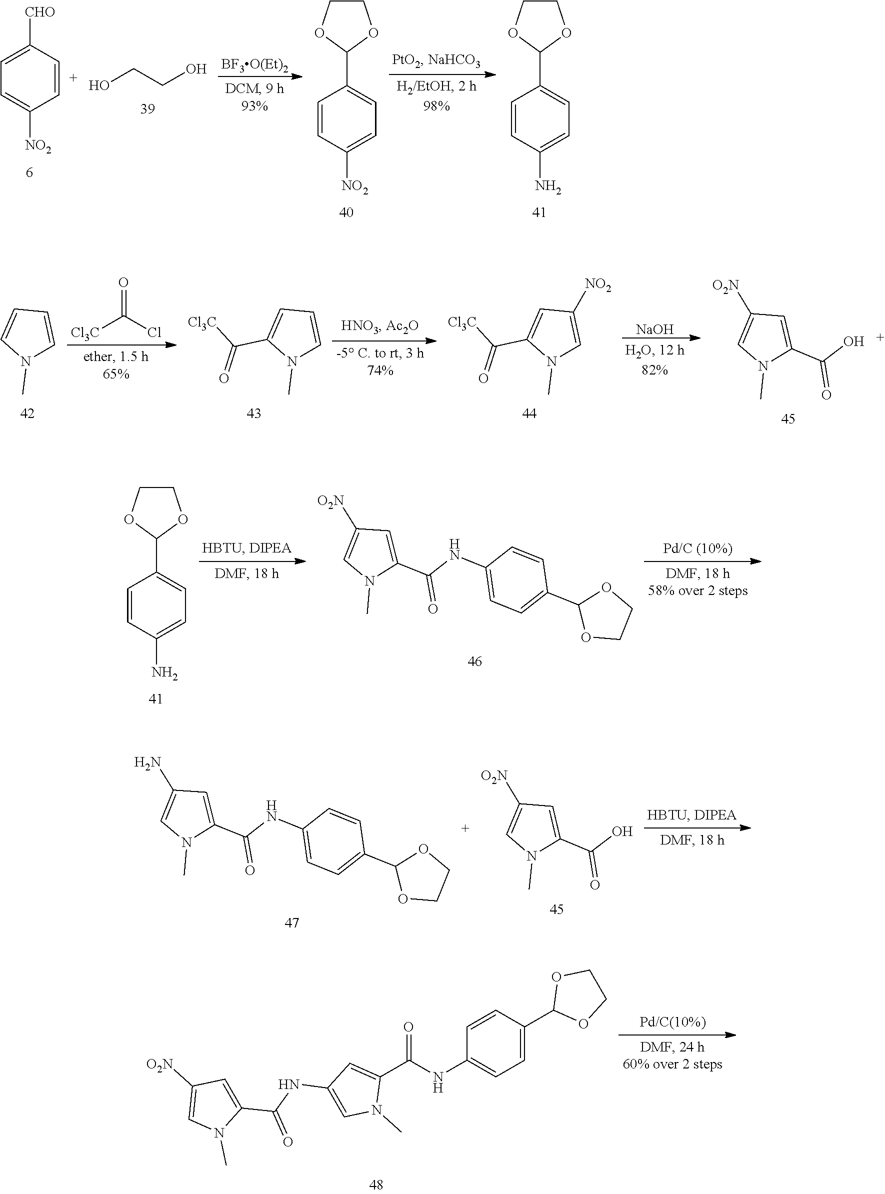

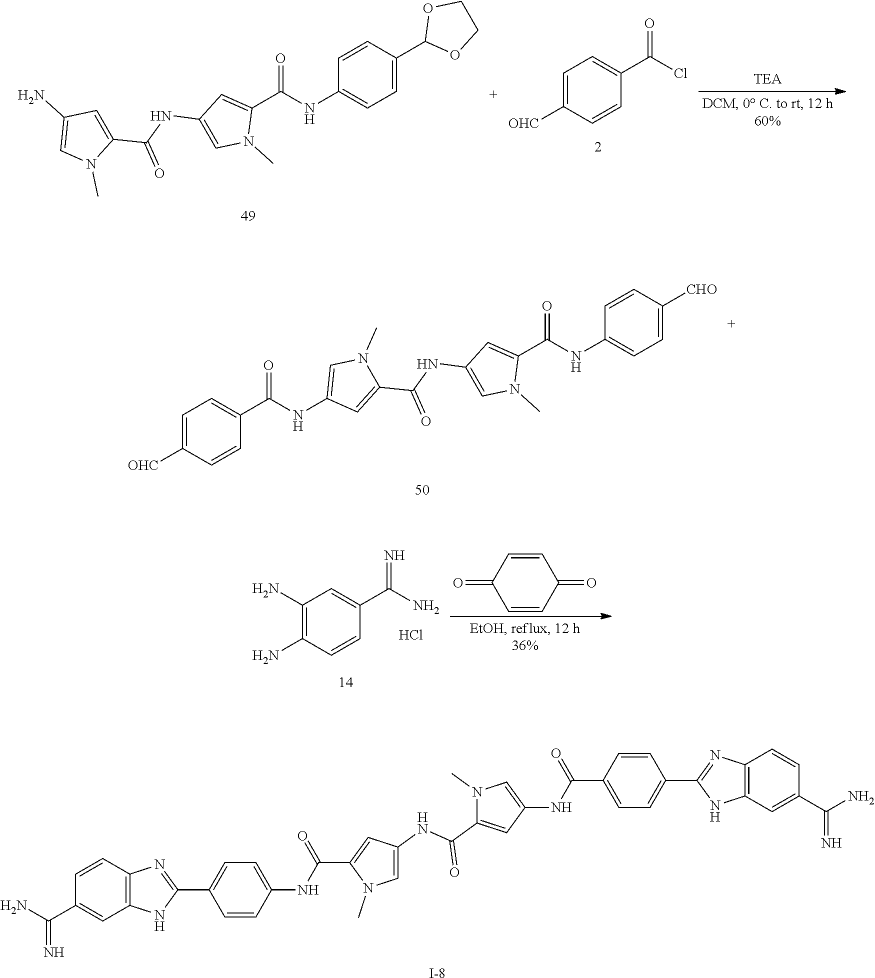

Step 1: Synthesis of 2-(4-nitrophenyl)-1,3-dioxolane (compound 40). To a solution of 4-nitrobenzaldehyde 6 (3.46 g, 22.90 mmol) in DCM (90 mL) was added ethane-1,2-diol 39 (6.6 mL, 0.12 mol), followed by trifluoroboron. etherate (0.6 mL). After stirring at room temperature for 9 h, the solution was washed with 10% NaOH, water, and brine. The resulting bright yellow solution was dried over anhydrous Na₂SO₄, filtered, and concentrated in vacuo to obtain the desired product 40 as a yellow solid (4.14 g, 93%).

¹H NMR (400 MHz, CDCl₃) δ 8.24 (d, J=8.8 Hz, 2H), 7.66 (d, J=8.6 Hz, 2H), 5.90 (s, 1H), 4.14-4.05 (m, 4H).

Step 2: Synthesis of 4-(1,3-dioxolan-2-yl)aniline (compound 41). To a mixture of PtO₂ (565 mg, 2.49 mmol) and NaHCO₃ (1.05 g, 12.50 mmol) was added a solution of 40 (2.45 g, 12.55 mmol) in anhydrous EtOH (150 mL). The flask was then evacuated, flushed three times with H₂, filled with H₂, and stirred at room temperature for 2 h. The reaction mixture was then filtered through a pad of celite, and washed with MeOH. The filtrate was concentrated under reduced pressure to give the crude product, which was dissolved in DCM and washed with water. The organic layer was dried over anhydrous Na₂SO₄, filtered, and concentrated in vacuo to give the desired product 41 as a light yellow oil (2.03 g, 98%).

¹H NMR (400 MHz, CDCl₃) δ 7.28-7.26 (m, 2H), 6.70-6.66 (m, 2H), 5.70 (s, 1H), 4.15-4.10 (m, 214), 4.03-3.98 (m, 2H), 3.72 (s, 2H).

Step 3: Synthesis of 2,2,2-trichloro-1-(1-methyl-1H-pyrrol-2-yl)ethan-1-one (compound 43). To a solution of 2,2,2-trichloroacetyl chloride (16.45 g, 90.47 mmol) in dry ether (25 mL) was added a solution of 1-methyl-1H-pyrrole 42 (7.34 g, 90.48 mmol) in dry ether (25 mL) dropwise. The reaction mixture was allowed to stir at room temperature for 1.5 h. Then the mixture was quenched with K₂CO₃ solution (20 mL, 20 mmol) dropwise, extracted with EtOAc, and the combined organic layers were dried over anhydrous Na₂SO₄, filtered, and concentrated in vacuo to give the crude product, which was washed with hexane and dried under vacuum to afford the desired product 43 as a white solid (13.24 g, 65%).

¹H NMR (400 MHz, CDCl₃) δ 7.51 (dd, J=4.4, 1.5 Hz, 1H), 6.97 (s, 1H), 6.23 (dd, J=4.4, 2.4 Hz, 1H), 3.98 (s, 3H).

Step 4: Synthesis of 2,2,2-trichloro-1-(1-methyl-4-nitro-1H-pyrrol-2-yl)ethan-1-one (compound 44). Fuming nitric acid (4 mL) was added dropwise to a stirred solution of 43 (10.67 g, 47.11 mmol) in Ac₂O (50 mL) which was maintained at −5° C. using an ice/NaCl bath. After addition was complete, the temperature was raised gradually to room temperature and stirred for an additional 3 h. The reaction mixture was then poured onto ice water (200 mL) and extracted with EtOAc. The combined organic layers were dried over anhydrous Na₂SO₄, filtered, and concentrated in vacuo. The crude product was purified by silica gel chromatography (10-50% EtOAc in petroleum ether) to give the desired product 44 as a light yellow solid (9.46 g, 74%).

¹H NMR (400 MHz, CDCl₃) δ 7.94 (d, J=1.7 Hz, 1H), 7.75 (d, J=1.3 Hz, 1H), 4.05 (s, 3H).

Step 5: Synthesis of 1-methyl-4-nitro-1H-pyrrole-2-carboxylic acid (compound 45). To a solution of NaOH (1.37 g, 34.25 mmol) in water (60 mL) was added 44 (3.10 g, 11.42 mmol), and the mixture was stirred at room temperature for 12 h. After completion of the reaction, the reaction mixture was extracted with EtOAc, and the aqueous layer was acidified with 2 M HCl until pH=3. The resulting solid was filtered and dried under vacuum to afford the desired product 45 as a white solid (1.60 g, 82%). 1H NMR (400 MHz, CDCl₃) δ 7.65 (d, J=1.2 Hz, 1H), 7.57 (d, J=1.8 Hz, 1H), 4.01 (s, 3H).

Step 6: Synthesis of N-(4-(1,3-dioxolan-2-yl)phenyl)-1-methyl-4-nitro-1H-pyrrole-2-carboxamide (compound 46). To a stirred solution of 45 (761 mg, 4.47 mmol) and HBTU (2.04 g, 5.38 mmol) in DMF (20 mL) was added DIPEA (1.5 mL, 9.08 mmol). After stirring at room temperature for 10 min, 41 (739 mg, 4.47 mmol) was added, and the mixture was stirred for an additional 18 h. After removal of the solvent, the residue was dissolved in CHCl₃/i-PrOH (3:1), and washed with water. The organic layer was then dried over anhydrous Na₂SO₄, filtered, and concentrated in vacuo to obtain a yellow solid 46 (1.42 g), which was used in the next step without further purification.

Step 7: Synthesis of N-(4-(1,3-dioxolan-2-yl)phenyl)-4-amino-1-methyl-1H-pyrrole-2-carboxamide (compound 47). To a solution of 46 (1.42 g, 4.47 mmol) in DMF (50 mL) was added Pd/C (1.42 g, 10%). The flask was then evacuated, flushed three times with 112, filled with 112, and stirred at room temperature for 18 h. The reaction mixture was filtered through a pad of celite, and washed with MeOH. The filtrate was concentrated under reduced pressure to give the crude product, which was purified by silica gel chromatography (0.5-1% MeOH in DCM) to give the desired product 47 as a light yellow solid (741.3 mg, 58% for 2 steps).

¹H NMR (400 MHz, CDCl₃) δ 7.56 (dd, J=5.2, 3.2 Hz, 3H), 7.44 (d, J=8.5 Hz, 2H), 6.34 (d, J=2.0 Hz, 1H), 6.24 (d, J=2.0 Hz, 1H), 5.78 (s, 1H), 4.14-4.09 (m, 2H), 4.05-4.00 (m, 2H), 3.85 (s, 3H), 2.93 (s, 2H).

Step 8: Synthesis of N-(4-(1,3-dioxolan-2-yl)phenyl)-1-methyl-4-(1-methyl-4-nitro-1H-pyrrole-2-carboxamido)-1H-pyrrole-2-carboxamide (compound 48). To a stirred solution of 45 (207 mg, 1.22 mmol) and HBTU (555 mg, 1.46 mmol) in DMF (15 mL) was added DIPEA (0.5 mL, 3.03 mmol). After stirring at room temperature for 10 min, 47 (350 mg, 1.22 mmol) was added, and the mixture was stirred for an additional 18 h. After removal of the solvent, the residue was dissolved in CHCl₃/i-PrOH (3:1), and washed with water. The organic layer was then dried over anhydrous Na₂SO₄, filtered, and concentrated in vacuo to obtain a yellow solid 48 (530 mg), which was used in the next step without further purification.

Step 9: Synthesis of N-(4-(1,3-dioxolan-2-yl)phenyl)-4-(4-amino-1-methyl-1H-pyrrole-2-carboxamido)-1-methyl-1H-pyrrole-2-carboxamide (compound 49). To a solution of 48 (530 mg, 1.21 mmol) in DMF (50 mL) was added Pd/C (800 mg, 10%). The flask was then evacuated, flushed three times with H₂, filled with H₂, and stirred at room temperature for 24 h. The reaction mixture was filtered through a pad of celite, and washed with MeOH. The filtrate was concentrated under reduced pressure to give the crude product, which was purified by silica gel chromatography (2-5% MeOH in DCM) to give the desired product 49 as a yellow solid (298.2 mg, 60% for 2 steps).

¹H NMR (400 MHz, CDCl₃) δ 7.62 (s, 1H), 7.58 (d, J=8.6 Hz, 2H), 7.46 (d, J=8.5 Hz, 2H), 7.34 (s, 1H), 7.14 (d, J=1.7 Hz, 1H), 6.76 (d, J=1.8 Hz, 1H), 6.35 (d, J=2.0 Hz, 1H), 6.18 (d, J=2.0 Hz, 1H), 5.80 (s, 1H). 4.15-4.12 (m, 2H), 4.07-4.01 (m, 2H), 3.94 (s, 3H), 3.87 (s, 3H), 2.97 (s, 2H).

Step 10: Synthesis of 4-(4-formylbenzamido)-N-(5-((4-formylphenyl)carbamoyl)-1-methyl-1H-pyrrol-3-yl)-1-methyl-1H-pyrrole-2-carboxamide (compound 50). To a solution of 49 (100 mg, 0.24 mmol) in DCM (6 mL) and TEA (60 μL) was added a solution of 2 (41 mg, 0.24 mmol) in DCM (6 mL) at 0° C. slowly. Then the mixture was warmed to room temperature and stirred for another 12 h. After removal of the solvent, the residue was dissolved in EtOAc, washed with aqueous HCl (1 M, 3×20 mL) and saturated aqueous NaHCO₃solution. The organic fractions were then dried over anhydrous Na₂SO₄, filtered, and concentrated in vacuo. The crude product was purified by silica gel chromatography (2-5% MeOH in DCM) to give the desired product 50 as a light yellow solid (72.8 mg, 60% for 2 steps).

¹H NMR (400 MHz, DMSO-d₆) δ 10.60 (s, 1H), 10.27 (s, 1H), 10.11 (s, 1H), 10.08 (s, 1H), 9.89 (s, 1H), 8.13 (d, J=8.2 Hz, 2H), 8.05 (d, J=8.2 Hz, 2H), 7.99 (d, J=8.6 Hz, 2H), 7.87 (d, J=8.6 Hz, 2H), 7.39-7.36 (m, 2H), 7.27 (d, J=1.5 Hz, 1H), 7.15 (d, J=1.5 Hz, 1H), 3.90 (s, 3H), 3.88 (s, 3H).

Step 11: Synthesis of 4-(4-(6-carbamimidoyl-1H-benzo[d]imidazol-2-yl)benzamido)-N-(5-((4-(6-carbamimidoyl-1H-benzo[d]imidazol-2-yl)phenyl)carbamoyl)-1-methyl-1H-pyrrol-3-yl)-1-methyl-1H-pyrrole-2-carboxamide (compound I-8). A solution of 50 (33.4 mg, 0.067 mmol), 3,4-diaminobenzimidamide hydrochloride 14 (25 mg, 0.13 mmol) and p-benzoquinone (14.6 mg, 0.13 mmol) in anhydrous EtOH (6 mL) was heated under reflux for 12 h. The reaction mixture was cooled to room temperature, and stirred in acetone (30 mL) for 0.5 h. The mixture was filtered, washed with dry ether, and dried to give a brown solid. Then the solid was dissolved in a 1:1 mixture of hot MeOH (5 mL) and EtOH (5 mL), filtered, the volume was reduced to 4 mL and acidified with HCl-saturated EtOH (0.6 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in 1120 with 0.05% HCl) to give the desired product 1-8 as a brown solid (21.9 mg, 36%).

¹H NMR (500 MHz, DMSO-d₆) δ 10.68 (s, 1H), 10.38 (s, 1H), 10.11 (s, 1H), 9.63 (s, 2H), 9.54 (s, 2H), 9.35 (s, 2H), 9.28 (s, 2H), 8.56 (d, J 8.0 Hz, 2H), 8.50 (d, J=8.4 Hz, 2H), 8.29 (d, J=5.4 Hz, 2H), 8.24 (d, J=8.0 Hz, 2H), 8.10 (d, J=8.4 Hz, 2H), 7.99-7.89 (m, 3H), 7.84 (d, J=8.4 Hz, 1H), 7.45-7.31 (m, 3H), 7.23 (d, J=10.2 Hz, 1H), 3.90 (d, J==4.5 Hz, 6H).

### Example S9: Synthesis of 4-(3-(4-(6-carbamimidoyl-1H-benzo[d]imidazol-2-yl)benzamido)propanamido)-N-(4-(6-carbamimidoyl-1H-benzo[d]imidazol-2 yl)phenyl)-1-methyl-1H-pyrrole-2-carboxamide (Compound 1-9)

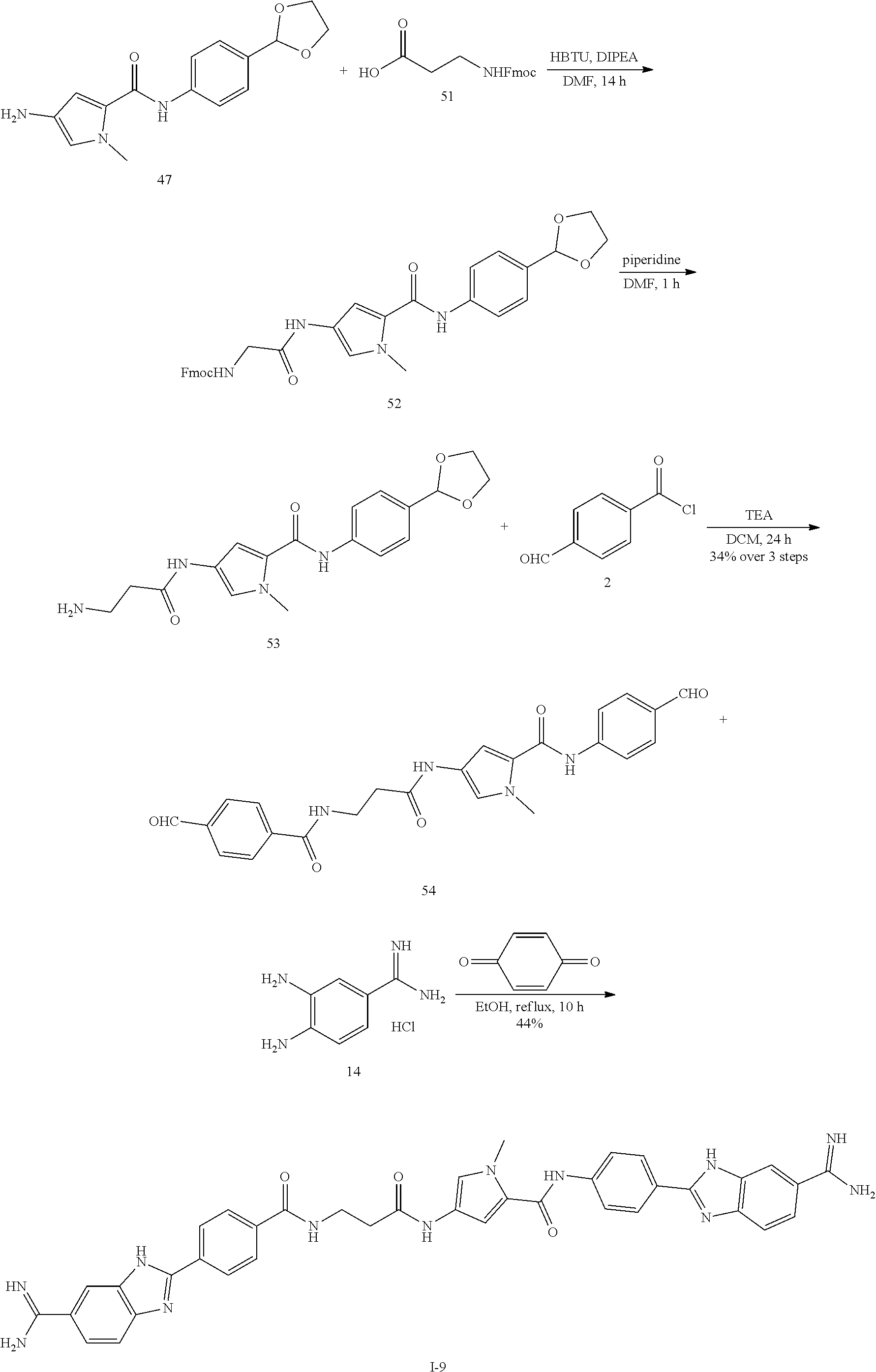

Step 1: Synthesis of (9H-fluoren-9-yl)methyl (3-((5-((4-(1,3-dioxolan-2-yl)phenyl)carbamoyl)-1-methyl-1H-pyrrol-3-yl)amino)-3-oxopropyl)carbamate (compound 52). To a stirred solution of Fmoc-β-alanine 51 (115 mg, 0.37 mmol) and HBTU (167 mg, 0.44 mmol) in DMF (20 mL) was added DIPEA (0.2 mL, 1.21 mmol). After stirring at room temperature for 10 min, 47 (105 mg, 0.37 mmol) was added, and the mixture was stirred for an additional 14 h. After removal of the solvent, the residue was dissolved in CHCl₃/i-PrOH (3:1), and washed with water. The organic layer was then dried over anhydrous Na₂SO₄, filtered, and concentrated in vacuo to obtain an orange solid 52 (212 mg), which was used in the next step without further purification.

Step 2: Synthesis of N-(4-(1,3-dioxolan-2-yl)phenyl)-4-(3-aminopropanamido)-1-methyl-1H-pyrrole-2-carboxamide (compound 53). To a solution of 52 (212 mg, 0.36 mmol) in DMF (10 mL) was added piperidine (0.34 mL), and the reaction mixture was stirred at room temperature for 1 h. After removal of the solvent, the crude residue 53 (130 mg) was used in the next step without further purification.

Step 3: Synthesis of 4-(3-(4-formylbenzamido)propanamido)-N-(4-formylphenyl)-1-methyl-1H-pyrrole-2-carboxamide (compound 54). To a solution of 53 (130 mg, 0.36 mmol) in DCM (9 mL) and TEA (90 μL) was added a solution of 2 (62 mg, 0.37 mmol) in DCM (9 mL) at 0° C. slowly. Then the mixture was warmed to room temperature and stirred for another 24 h. After removal of the solvent, the residue was dissolved in EtOAc, washed with aqueous HCl (1 M, 3×20 mL) and saturated aqueous NaHCO₃ solution. The organic fractions were then dried over anhydrous Na₂SO₄, filtered, and concentrated in vacuo. The crude product was purified by silica gel chromatography (0-2% MeOH in DCM) to give the desired product 54 as a white solid (54.8 mg, 34% for 3 steps).

¹H NMR (400 MHz, DMSO-d₆) δ 10.21 (s, 1H), 10.07 (s, 1H), 10.00 (s, 1H), 9.88 (s, 1H), 8.83 (t, J=5.4 Hz, 1H), 8.04-7.95 (m, 6H), 7.86 (d, J=8.7 Hz, 2H), 7.29 (d, J=1.5 Hz, 1H), 7.06 (d, J=1.5 Hz, 1H), 3.85 (s, 3H), 3.57 (dd, J=12.7, 6.8 Hz, 2H), 2.59 (t, J=7.0 Hz, 2H).

Step 4: Synthesis of 4-(3-(4-(6-carbamimidoyl-1H-benzo[d]imidazol-2-yl)benzamido)propanamido)-N-(4-(6-carbamimidoyl-1H-benzo[d]imidazol-2-yl)phenyl)-1-methyl-1H-pyrrole-2-carboxamide (Compound 1-9). A solution of 54 (80 mg, 0.18 mmol), 3,4-diaminobenzimidamide hydrochloride 14 (67 mg, 0.36 mmol) and p-benzoquinone (39 mg, 0.36 mmol) in anhydrous EtOH (7 mL) was heated under reflux for 10 h. The reaction mixture was cooled to room temperature, and stirred in acetone (40 mL) for 0.5 h. The mixture was filtered, washed with dry ether, and dried to give a brown solid. Then the solid was dissolved in a 1:1 mixture of hot MeOH (15 mL) and EtOH (15 mL), filtered, the volume was reduced to 10 mL and acidified with HCl-saturated EtOH (1 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in I120 with 0.05% HCl) to give the desired product I-9 as a brown solid (68 mg, 44%).

¹H NMR (500 MHz, DMSO-d₆) δ 10.34 (s, 1H), 10.17 (s, 1H), 9.64 (s, 2H), 9.54 (s, 2H), 9.34 (s, 2H), 9.26 (s, 2H), 8.89 (t, J=5.5 Hz, 1H), 8.46 (dd, J=11.2, 8.4 Hz, 4H), 8.27 (s, 2H), 8.11 (d, J=8.2 Hz, 2H), 8.07 (d, J=8.6 Hz, 2H), 7.94 (d, J=8.6 Hz, 1H), 7.89 (d, J=8.5 Hz, 2H), 7.82 (d, 0.1-8.5 Hz, 1H), 7.31 (s, 1H), 7.16 (s, 1H), 3.86 (s, 3H), 3.60 (q, J=6.7 Hz, 2H), 2.65 (t, J=7.2 Hz, 2H).

### Example S10: Synthesis of (S)-1-((4-(6-carbamimidoyl-5-methoxy-1H-benzo[d]imidazol-2-yl)benzoyl)-L prolyl)-N-(4-(6-carbamimidoyl-S-methoxy-1H-benzo[d]imidazol-2-yl)phenyl)pyrrolidine-2-carboxamide (Compound 1-10)

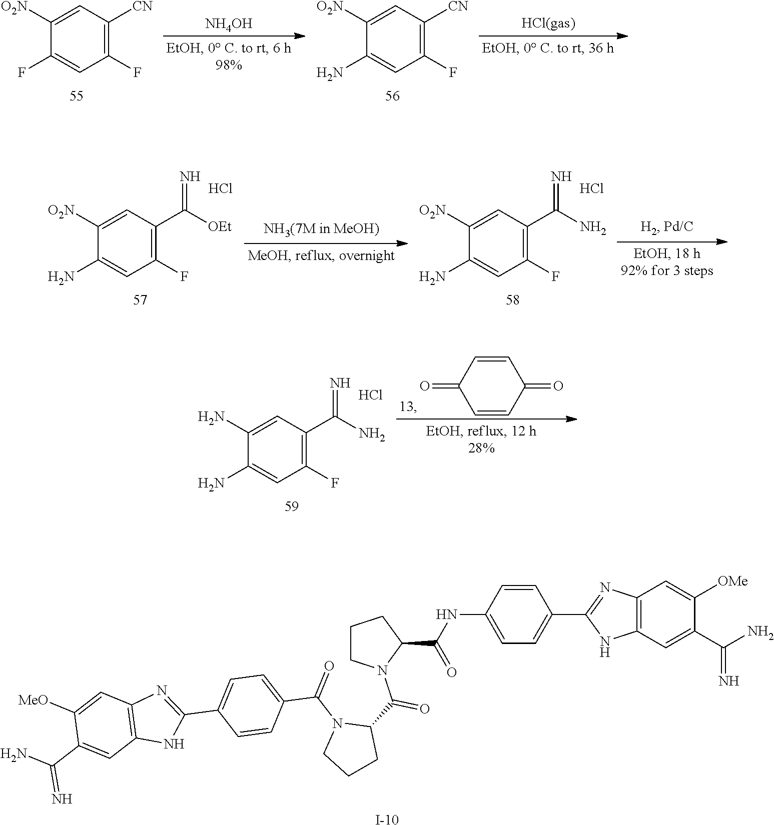

Step 1: Synthesis of 4-amino-2-fluoro-5-nitrobenzonitrile (compound 56). To a solution of 2,4-difluoro-5-nitrobenzonitrile 55 (2.2 g, 11.95 mmol) in EtOH (1.5 mL) at 0° C. was added NH₄OH (6.5 mL), and the resulting mixture was stirred at room temperature for 6 h. The resulting precipitate was then filtered and dried under vacuum to give the desired product 56 as a yellow solid (2.21 g, 98%).

¹H NMR (400 MHz, DMSO-d₆) δ 8.60 (d, J=7.0 Hz, 1H), 8.24 (s, 2H), 6.88 (d, J=11.9 Hz, 1H).

Step 2: Synthesis of ethyl 4-amino-2-fluoro-5-nitrobenzimidate hydrochloride (compound 57). Dry HCl gas was passed through a stirred suspension of 56 (1.45 g, 8.00 mmol) in EtOH (40 mL) until the reaction mixture was saturated with HCl, and the mixture was stirred at room temperature for 36 h. The reaction mixture was then diluted with dry ether. The imidate ester was precipitated as an orange solid, filtered, washed with ether, and dried under vacuum to obtain an orange solid 57 (1.88 g), which was used in the next step without further purification.

Step 3: Synthesis of 4-amino-2-methoxy-5-nitrobenzimidamide hydrochloride (compound 58). To a stirred suspension of 57 (278 mg, 1.05 mmol) in MeOH (3 mL) was added NH₃ (7 M in MeOH, 3 mL), and the reaction mixture was refluxed overnight. The reaction mixture was then concentrated in vacuo, and diluted with ether. The resulting precipitate was filtered, washed with ether, and dried under vacuum to yield a yellow solid 58 (306.9 mg), which was used in the next step without further purification.

Step 4: Synthesis of 4,5-diamino-2-methoxybenzimidamide hydrochloride (compound 59). To a solution of 58 (170 mg, 1.32 mmol) in EtOH (10 mL) was added Pd/C (20 mg, 10%). The flask was then evacuated, flushed three times with H₂, filled with H₂, and stirred at room temperature for 18 h. The reaction mixture was filtered through a pad of celite, and washed with MeOH. The filtrate was concentrated under reduced pressure to give the desired product 59 as a yellow solid (130.2 mg, 92% for 3 steps).

¹H NMR (400 MHz, Methanol-d₄) δ 7.02 (s, 1H), 6.48 (s, 1H), 3.87 (s, 3H).

Step 5: Synthesis of (S)-1-((4-(6-carbamimidoyl-5-methoxy-1H-benzo[d]imidazol-2-yl)benzoyl)-L-prolyl)-N-(4-(6-carbamimidoyl-5-methoxy-1H-benzo[d]imidazol-2-yl)phenyl)pyrrolidine-2-carboxamide (compound I-10). A solution of 13 (52 mg, 0.12 mmol), 4,5-diamino-2-methoxybenzimidamide hydrochloride 59 (50 mg, 0.23 mmol) and p-benzoquinone (25 mg, 0.23 mmol) in anhydrous EtOH (5 mL) was heated under reflux for 12 h. The reaction mixture was cooled to room temperature, and stirred in acetone (50 mL) for 0.5 h. The mixture was filtered, washed with dry ether, and dried to give a brown solid. Then the solid was dissolved in a 1:1 mixture of hot MeOH (10 mL) and EtOH (10 mL), filtered, the volume was reduced to 7 mL and acidified with HCl-saturated EtOH (0.7 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-10 as a brown solid (29.8 mg, 28%).

¹H NMR (400 MHz, Methanol-d₄) δ 8.23 (d, J=8.2 Hz, 2H), 8.15 (d, J=8.9 Hz, 2H), 8.03-7.97 (m, 4H), 7.88 (d, J=8.2 Hz, 2H), 7.49 (d, J=15.1 Hz, 2H), 4.96 (dd, J=8.3, 5.8 Hz, 1H), 4.68 (dd, J=8.3, 4.7 Hz, 1H), 4.06 (s, 3H), 4.05 (s, 3H), 4.04-3.98 (m, 1H), 3.86-3.76 (m, 1H), 3.72-3.58 (m, 2H), 2.56-2.45 (m, 1H), 2.44-2.35 (m, 1H), 2.27-1.92 (m, 6H).

### Example S11: Synthesis of (S)-1-((4-(6-carbamimidoyl-5 fluoro-1H-benzo[d]imidazol-2-yl)benzoyl)-L-prolyl)-N-(4-(6-carbamimidoyl-5-fluoro-1H-benzo[d]imidazol-2-yl)phenyl)pyrrolidine-2-carboxamide (Compound 1-11)

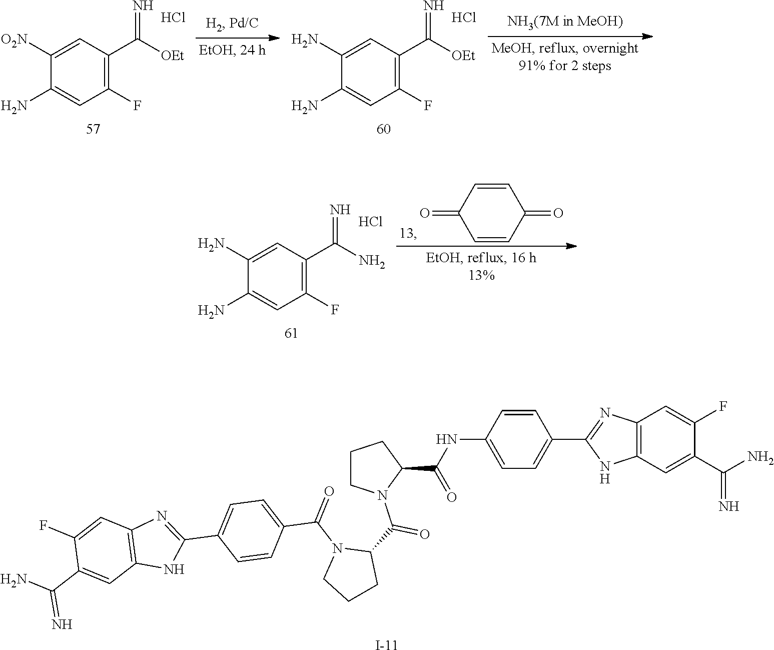

Step 1: Synthesis of ethyl 4,5-diamino-2-fluorobenzimidate hydrochloride (compound 60). To a solution of 57 (350 mg, 1.33 mmol) in EtOH (30 mL) was added Pd/C (40 mg, 10%). The flask was then evacuated, flushed three times with H₂, filled with H₂, and stirred at room temperature for 24 h. The reaction mixture was filtered through a pad of celite, and washed with MeOH. The filtrate was concentrated under reduced pressure to obtain an orange solid 60 (323.4 mg), which was used in the next step without further purification.

Step 2: Synthesis of 4,5-diamino-2-fluorobenzimidamide hydrochloride (compound 61). To a suspension of 60 (320 mg, 1.37 mmol) in MeOH (15 mL) was added NH₃ (7 M MeOH, 2 mL), and the reaction mixture was refluxed overnight. The reaction mixture was then concentrated in vacuo, and diluted with ether. The resulting precipitate was filtered, washed with ether, and dried under vacuum to give the desired product 61 as a reddish brown solid (248.1 mg, 91% for 2 steps).

¹H NMR (400 MHz, Methanol-d₄) δ 6.91 (d, J=7.1 Hz, 1H), 6.50 (d, J=13.5 Hz, 1H).

Step 3: Synthesis of (S)-1-((4-(6-carbamimidoyl-5-fluoro-1H-benzo[d]imidazol-2-yl)benzoyl)-L-prolyl)-N-(4-(6-carbamimidoyl-5-fluoro-1H-benzo[d]imidazol-2-yl)phenyl)pyrrolidine-2-carboxamide (compound I-11). A solution of 13 (67.1 mg, 0.15 mmol), 4,5-diamino-2-fluorobenzimidamide hydrochloride 61 (61.4 mg, 0.30 mmol) and p-benzoquinone (32.7 mg, 0.30 mmol) in anhydrous EtOH (12 mL) was heated under reflux for 16 h. The reaction mixture was cooled to room temperature, and stirred in acetone (80 mL) for 0.5 h. The mixture was filtered, washed with dry ether, and dried to give a brown solid. Then the solid was dissolved in a 1:1 mixture of hot MeOH (13.2 mL) and EtOH (13.2 mL), filtered, the volume was reduced to 9 mL and acidified with HCl-saturated EtOH (1.8 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product I-11 as a brown solid (17.4 mg, 13%).

¹H NMR (400 MHz, Methanol-d₄) δ 8.31-8.18 (m, 6H), 8.06-7.85 (m, 6H), 5.00-4.95 (m, 1H), 4.69 (dd, J=8.3, 4.7 Hz, 1H), 4.05-3.98 (m, 1H), 3.87-3.76 (m, 1H), 3.71-3.56 (m, 2H), 2.57-2.47 (m, 1H), 2.46-2.35 (m, 1H), 2.29-1.93 (m, 6H).

### Example S12: Synthesis of (5)-1-((4-(6-carbamimidoyl-1-methyl-1H-benzo[d]imidazol-2-yl)benzoyl)-L-prolyl)-N-(4-(6-carbamimidoyl-1-methyl-1H-benzo[d]imidazol-2-yl)phenyl)pyrrolidine-2-carboxamide (Compound 1-12)

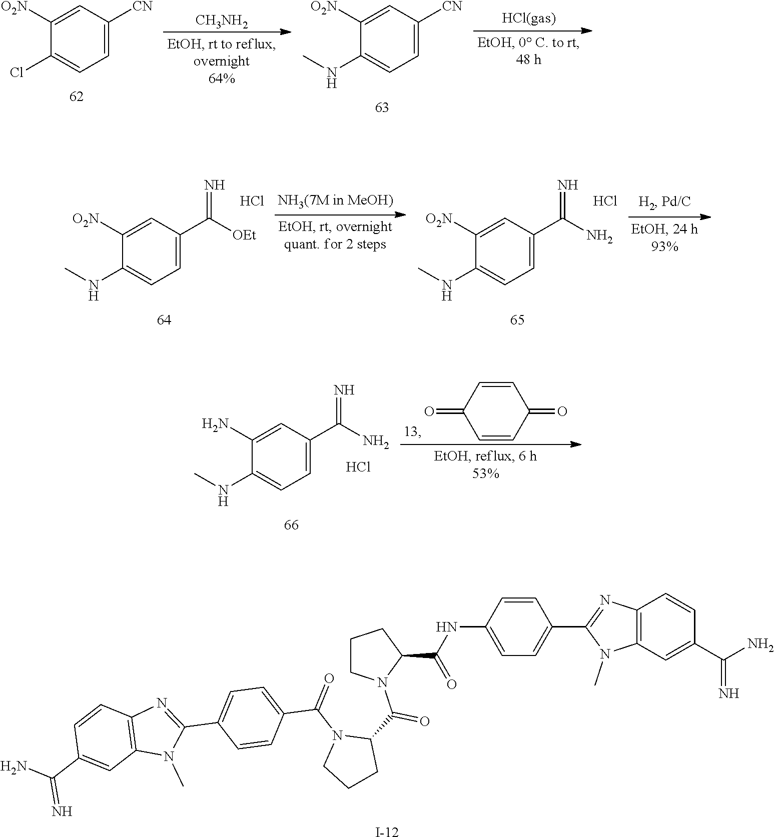

Step 1: Synthesis of 4-(methylamino)-3-nitrobenzonitrile (compound 63). To a suspended solution of 4-chloro-3-nitrobenzonitrile 62 (1.5 g, 8.22 mmol) in EtOH (6 mL) was added CH₃NH₂ (27-32% in EtOH, 1.5 mL), and the reaction mixture was stirred at room temperature for 1 h, then refluxed overnight. The reaction mixture was cooled and concentrated in vacuo. The residue was suspended in ether, and filtered to give the crude product, which was purified by silica gel chromatography (pure DCM) to give the desired product 63 as a yellow solid (936.3 mg, 64%).

¹H NMR (400 MHz, DMSO-d₆) δ 8.64 (d, J=6.8 Hz, 1H), 8.50 (d, J=2.0 Hz, 1H), 7.84 (ddd, J=9.0, 2.1, 0.8 Hz, 1H). 7.11 (d, J=9.1 Hz, 1H), 3.00 (d, J=5.0 Hz, 3H).

Step 2: Synthesis of ethyl 4-(methylamino)-3-nitrobenzimidate hydrochloride (compound 64). Dry HCl gas was passed through a stirred suspension of 63 (710 mg, 8.00 mmol) in EtOH (20 mL) cooled in an ice-salt bath until the reaction mixture was saturated with HCl, and the mixture was stirred at room temperature for 48 h. The reaction mixture was then diluted with dry ether. The imidate ester was precipitated as an orange solid, filtered, washed with ether, and dried under vacuum to obtain an orange solid 64 (1.08 g), which was used in the next step without further purification.

Step 3: Synthesis of 4-(methylamino)-3-nitrobenzimidamide hydrochloride (compound 65). To a suspension of 64 (1.08 g, 4.16 mmol) in MeOH (20 mL) was added NH₃ (7 M MeOH, 3 mL), and the reaction mixture was stirred overnight at room temperature. The reaction mixture was then concentrated in vacuo, and diluted with ether. The resulting precipitate was filtered, washed with ether, and dried under vacuum to give the desired product 65 as a yellow solid (945.8 mg, quant. for 2 steps).

¹H NMR (400 MHz, DMSO-d₆) δ 9.30 (s, 2H), 8.98 (s, 2H), 8.70 (d, J=2.4 Hz, 1H), 8.68 (d, J=5.2 Hz, 1H), 7.98 (dd, J=9.2, 2.4 Hz, 1H), 7.17 (d, J=9.3 Hz, 1H), 3.03 (d, J=5.0 Hz, 3H).

Step 4: Synthesis of 3-amino-4-(methylamino)benzimidamide hydrochloride (compound 66). To a solution of 65 (686.8 mg, 3.00 mmol) in EtOH (30 mL) was added Pd/C (70 mg, 10%). The flask was then evacuated, flushed three times with H₂, filled with H₂, and stirred at room temperature for 24 h. The reaction mixture was filtered through a pad of celite, and washed with MeOH. The filtrate was concentrated under reduced pressure to give the desired product 66 as a yellow solid (556.2 mg, 93%).

¹H NMR (400 MHz, DMSO-d₆) δ 8.74 (s, 2H), 8.42 (s, 2H), 7.12 (dd, J=8.3, 2.3 Hz, 1H), 6.92 (d, J=2.3 Hz, 1H), 6.47 (d, J=8.4 Hz, 1H), 5.76 (d, J=5.1 Hz, 1H), 4.88 (s, 2H), 2.80 (d, J=4.7 Hz, 3H).

Step 5: Synthesis of (S)-1-((4-(6-carbamimidoyl-1-methyl-11l-benzo[d]imidazol-2-yl)benzoyl)-L-prolyl)-N-(4-(6-carbamimidoyl-1-methyl-1H-benzo[d]imidazol-2-yl)phenyl)pyrrolidine-2-carboxamide (compound I-12). A solution of 13 (135.4 mg, 0.30 mmol), 3-amino-4-(methylamino)benzimidamide hydrochloride 66 (121.4 mg, 0.60 mmol) and p-benzoquinone (65.9 mg, 0.60 mmol) in anhydrous EtOH (12 mL) was heated under reflux for 6 h. The reaction mixture was cooled to room temperature, and stirred in acetone (100 mL) for 0.5 h. The mixture was filtered, washed with dry ether, and dried to give a hydrochloride salt. Then the solid was dissolved in a 1:1 mixture of hot MeOH (26 mL) and EtOH (26 mL), filtered, the volume was reduced to 18 mL and acidified with HCl-saturated EtOH (1.8 mL). After stirring overnight at room temperature, the mixture was diluted with ether, and the resulting precipitate was filtered, washed with ether, and dried under vacuum. The crude product was purified by preparative reverse-phase HPLC (5-100% acetonitrile in H₂O with 0.05% HCl) to give the desired product 1-12 as a pink solid (141.6 mg, 53%).

¹H NMR (400 MHz, Methanol-d₄) δ 8.30 (dd, J=6.9, 1.3 Hz, 2H), 8.14 (d, J=8.8 Hz, 1H), 8.06-7.99 (m, 6H), 7.98-7.89 (m, 5H), 4.98 (dd, J=8.2, 5.7 Hz, 1H), 4.70 (dd, J=8.2, 4.8 Hz, 1H), 4.16 (s, 3H), 4.10 (s, 3H), 4.07-3.99 (m, 1H), 3.87-3.78 (m, 1H), 3.74-3.60 (m, 2H), 2.57-2.47 (m, 1H), 2.46-2.36 (m, 1H), 2.30-1.96 (m, 6H).

### Biological Examples

### Example B1: Biological Evaluation of Potency of PU.1 Inhibitors

An acute T cell lymphoblastic leukemia (T-ALL) disease model was used to evaluate the effect of the newly synthesized compounds on PU.1 inhibition. Pten, a famous tumor suppressor gene, was 40% deleted in mouse hematopoietic stem cells and their differentiated progeny, and this would eventually lead to a progressive T-ALL in about two months from born. It is believed that immune checkpoint T cell immunoglobulin mucin-3 (TIM-3), a surface marker for the isolation of pure leukemia-initiating cells (LICs), is transcriptionally regulated by transcription factor PU.1 in the Pten-null T-ALL model. Therefore, the expression level of TIM-3 was detected and quantified to characterize the inhibition potency of the compounds after 24 h treatment of graded concentrations of compounds. Blast cells were transfected with either PU.1-EGFP-vector or EGFP-vector to yield stable cell lines blast-PU.1 and blast-EGFP, respectively. These cell lines were used for compound testing in vitro. Compounds DB1976 and DB2115 were tested as well for comparison. Blast-PU.1 and blast-EGFP are the ideal cell lines for compounds testing in vitro, since they are T-ALL blast cells with low TIM-3 and PU.1 expression level.

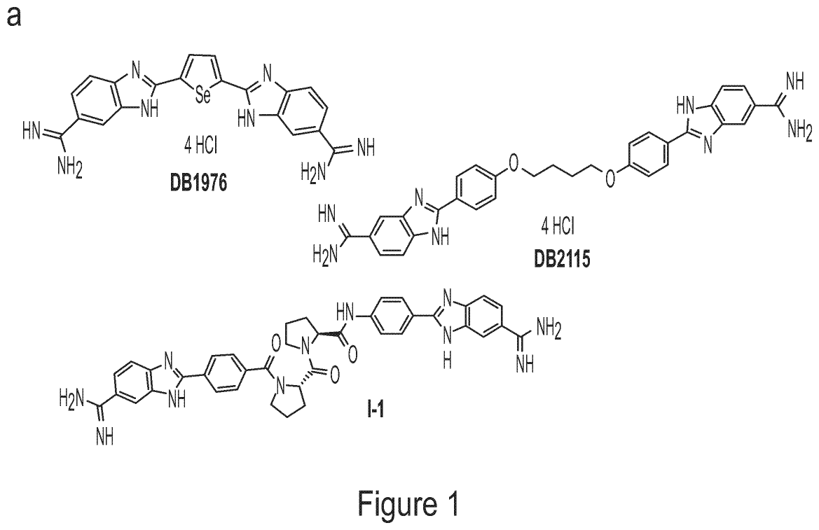

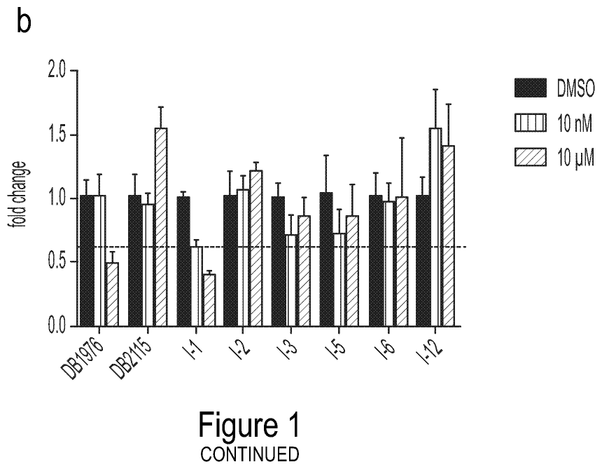

Replacement of the flexible alkyl linker of DB2115 with rigid L-prolines in compound I-1 led to a marked enhancement in potency, since compound I-1 downregulated the expression level of TIM-3 by 40% at 10 nM in Blast-PU.1 cell line (FIG. 1b), and TIM-3 level is too low to detect in both DMSO and compounds-treated groups (Data not shown). In contrast, its D, D-prolines analogue compound I-2 and D, L-prolines analogue compound I-3 displayed no obvious inhibition at 10 μM, yet not as good as compound I-1 (FIG. 1b). Taken together, compound I-1 exhibited the best activity in terms of PU.1-mediated TIM-3 inhibition, and we concluded that compound I-1 can be utilized for follow-up biologically functional investigations.

### Example B2: Effects of the Combination of Compound I-1 and Rapamycin on Reduction of Leukemia Progression

To generate a Pten-null T-ALL mouse model, Pten was 40% deleted in mouse fetal liver hematopoietic stem cells (HSCs), followed by PI3K-AKT pathway activation, hematopoietic disorder and T-ALL development. In the T-ALL crisis stage, T-ALL blasts and LICs would infiltrate mouse hematopoietic organs and non-hematopoietic organs.

T-ALL mice were treated with a combination of rapamycin, a well-studied PI3K-AKT pathway inhibitor that shows promising effects in targeting T-ALL blast cells, and compound I-1. Treatment was initiated at the blast crisis stage, and treatment was stopped after 62 days after birth to observe the immediate effects of the compounds in inhibiting both blast cells and TIM-3-high LICs.

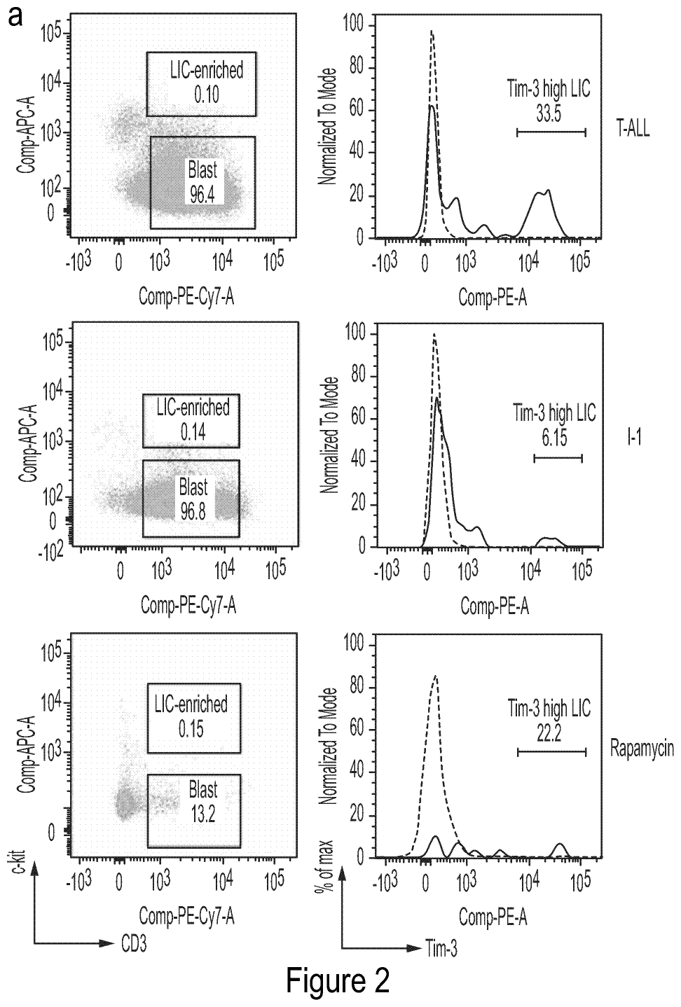

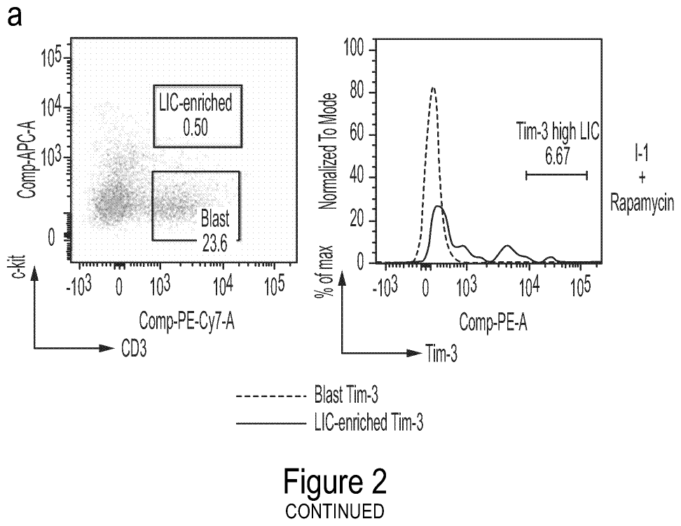

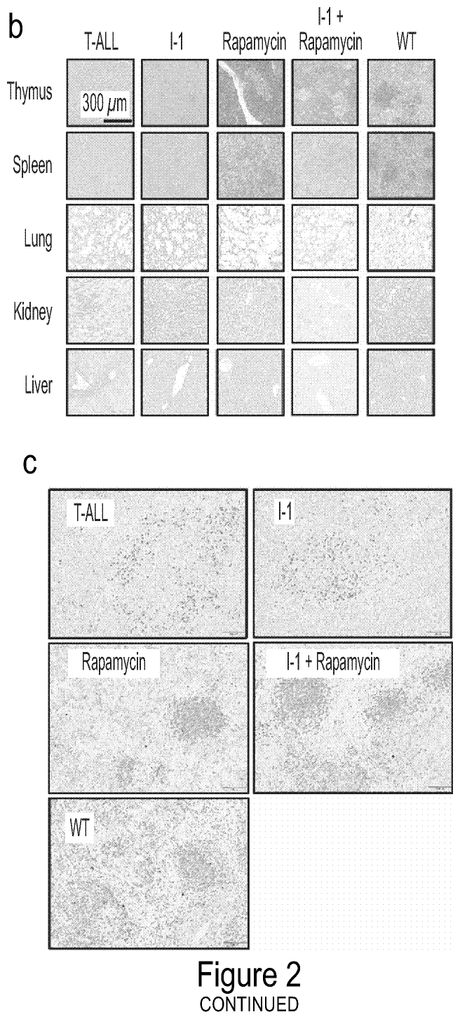

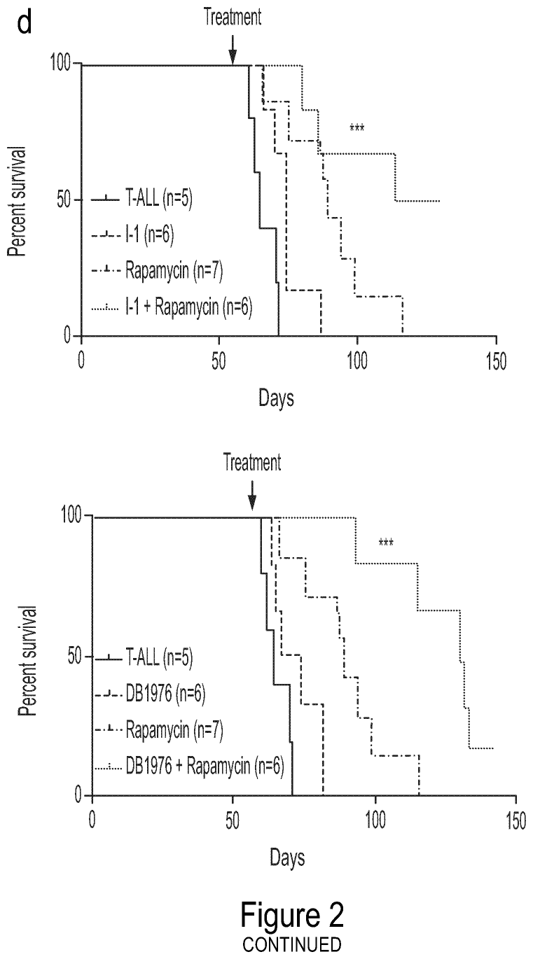

After two days' treatment, compound I-1 alone did not reduce the proportion of blasts and live lymphocytes, while rapamycin showed great efficiency in targeting blasts, leading to the decrease of blasts from 96.4% to 13.2% (FIG. 2a). Combination treatment gave rise to more significant reduction of blasts proportion in bone marrow (FIG. 2a), spleen and thymus. For TIM-3 high LIC population, treatment with compound I-1 alone remarkably reduced LICs proportion from 33.5% to 6.15%, in comparison to the rapamycin-treated group (22.2%). Importantly, combination treatment displayed marked effects in reducing both blasts and LICs compared to another three groups.

Previously, it has been shown that co-targeting blasts and LICs in a T-ALL mouse model using DB1976 and PI3K inhibitors can reduce tumor burden. To test whether compound I-1 can achieve this, T-ALL mice were treated at the blast crisis stage with compound I-1 and/or rapamycin for one month. After treatment, hematopoietic and non-hematopoietic organ morphology of the mice was analyzed using hematoxylin-eosin (H&E) staining. Organ morphology of the group treated with compound I-1 alone showed no significant changes compared with that of T-ALL group, indicating that targeting LICs alone did not reduce tumor burden, while rapamycin group showed improvement of the therapeutic effects as blast cells are the major population of leukemia cells. The morphology of thymus and spleen was recovered, and infiltration of leukemia cells into the lung, kidney and liver was significantly reduced in combination treatment group (FIG. 2b).

Studies have shown that after Pten deletion, B cells development is repressed at prepro-B stage. As shown in FIG. 2c, low amount of B220 positive cells were observed in T-ALL mice spleen; however, B cells population was rescued after combination treatment with compound I-1 and rapamycin in the mouse spleen B220 immunohistochemistry slides (FIG. 2c). In B lineage cells, PU.1 is a major regulator controlling lineage commitment, and PU.1 expression would increase gradually from pro-B to B cells. There is a possibility that expression levels of PU.1 and/or its downstream genes can return to normal in combination treatment group, and the lymphoid lineage commitment and cell differentiation proceed normally. Moreover, compound I-1 in combination with rapamycin prolonged mouse survival more than compound I-1 or rapamycin alone, or DB1976 in combination with rapamycin (FIG. 2d).

### Example B3: Preventive and Therapeutic Effects of Compound I-1 on Skin Fibrosis

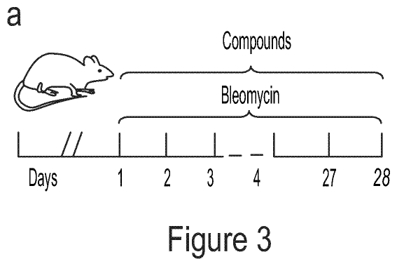

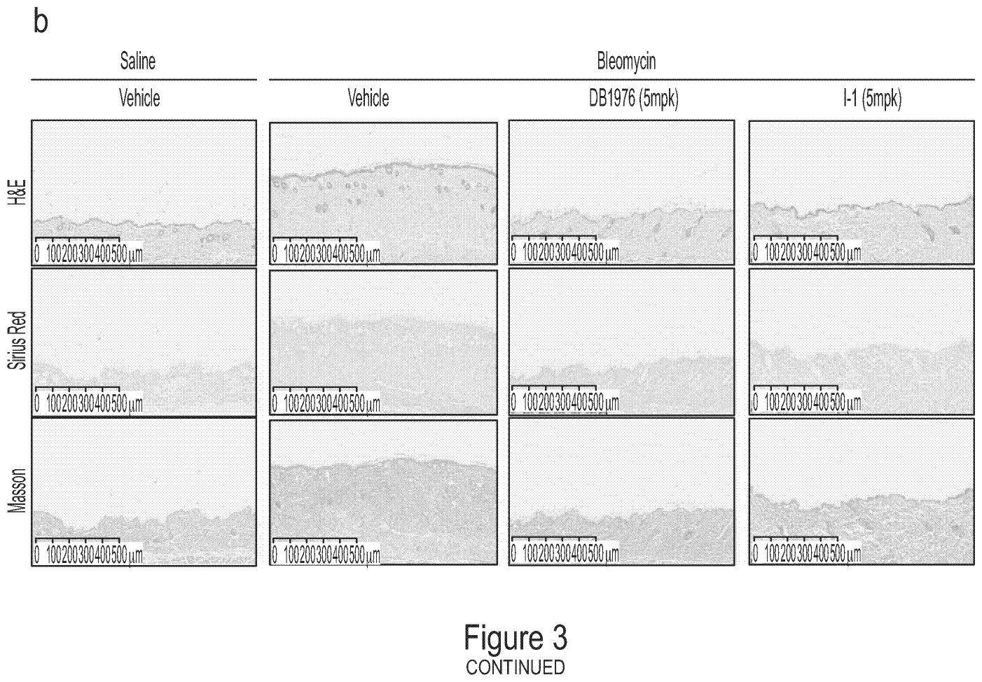

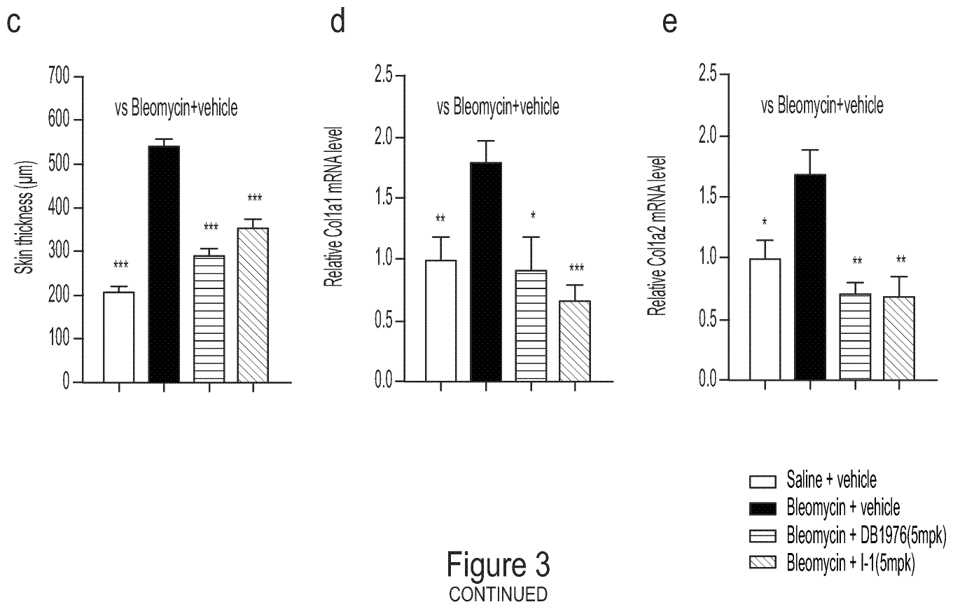

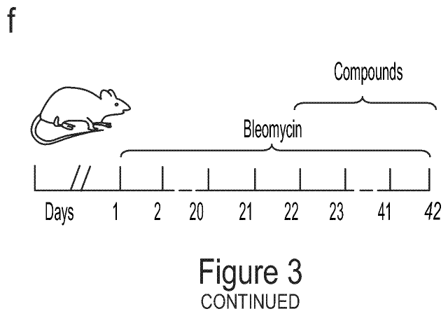

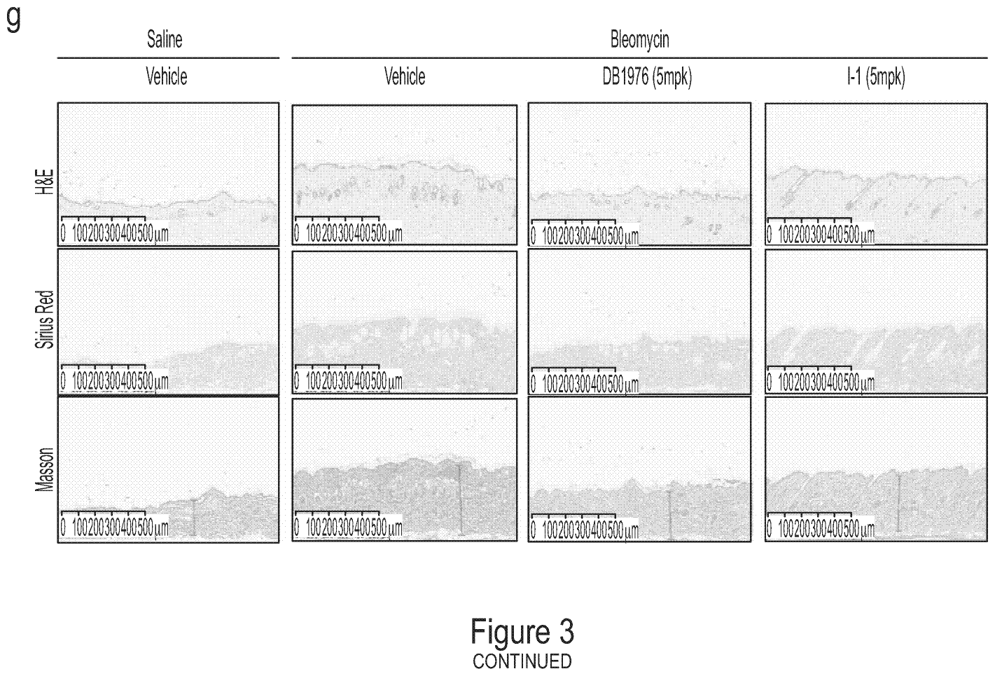

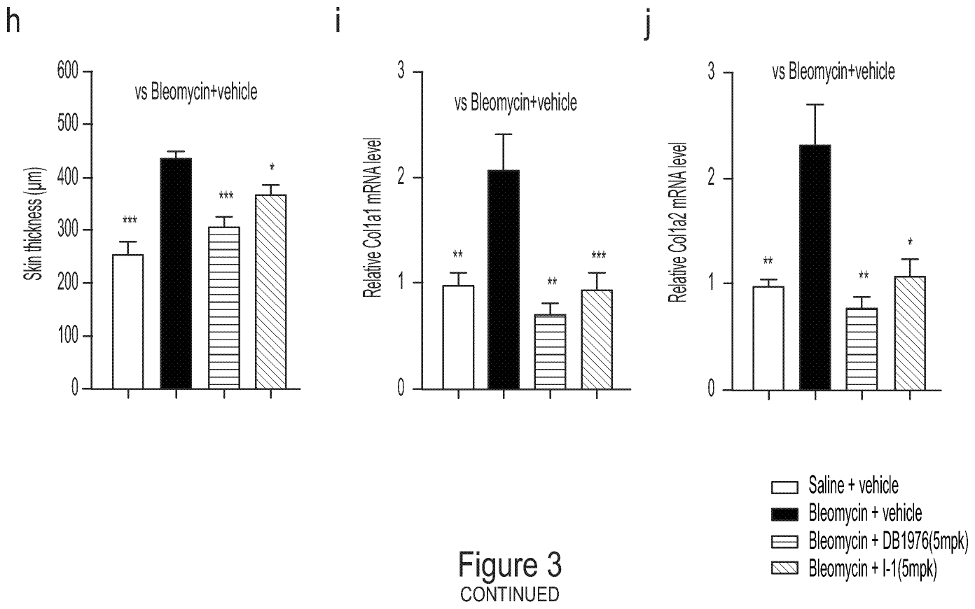

Methods: To evaluate the preventive and therapeutic effects of compound I-1 on skin fibrosis, bleomycin was used to establish two animal models of skin fibrosis with different drug intervention (6-8 weeks, C₅₇BL/6, male). Skin fibrosis was induced by local injection of bleomycin (0.5 mg/mL, 0.1 ml/mouse) in a skin defined area (˜1 cm²), where the hair was removed in advance, at the upper back every other day. Subcutaneous saline injections served as controls. (I) Preventive model of bleomycin-induced skin fibrosis: Compound I-1, positive control DB1976, or vehicle (saline) were injected i.p. simultaneously with bleomycin for 4 weeks (FIG. 3a). (II) Therapeutic model of bleomycin-induced skin fibrosis: Mice were pre-charged with bleomycin for 3 weeks to induce skin fibrosis, then treated with compound I-1, positive control DB1976, or vehicle (saline) for another 3 weeks, total time is 6 weeks after the first bleomycin treatment (FIG. 30. After the last day of treatment of both models, mice were fasted overnight and euthanized. Part of the skin was fixed with paraformaldehyde after fully flat on a foil, embedded with paraffin, and then sliced for H&E, Sirius red and Masson staining to check pathological features. The epidermal thickness of each sample was calculated quantitatively with image J. Another part of skin was collected and stored at −80° C. after liquid nitrogen quick-freezing, RNA isolation (Code.R₆₉₃₄, OMEGA, USA), first cDNA reverse (Code.AT341, TransGen, China), SYBR mix (Code.AQ601, TransGen, China) for Q-PCR (LightCycler® 96, Roche) to verify several fibrosis-related genes mRNA level, such as, Col1a1 and Col1a2.

Results: In the preventive model of bleomycin-induced skin fibrosis, bleomycin treatment for 4 weeks significantly induced skin fibrosis pathological features, including increasing skin epidermal thickness, collagen deposition, and increasing Col1a1 and Col1a2 mRNA levels when compared with saline/vehicle group, which indicated the successful establishment of the bleomycin-induced skin fibrosis model. Compared with the bleomycin/vehicle group, treatment with compound I-1 or DB1976 significantly prevented the skin fibrosis progress, reducing the epidermal thickness and collagen deposition, decreasing the mRNA level of Col1a1 and Col1a2 (FIG. 3a-3e). In the therapeutic model of bleomycin-induced skin fibrosis, bleomycin treatment for 6 weeks also significantly induced skin fibrosis pathological features, yielding thicker epidermal skin, more collagen deposition and higher mRNA levels of Col1a1 and Col1a2 when compared with saline/vehicle group, which further indicated the successful establishment of skin fibrosis model by bleomycin stimulation. Treatment with compound I-1 or DB1976 for 3 weeks significantly alleviated and reversed bleomycin-induced skin fibrosis (FIG. 3f-3j). These data suggest that compound I-1 has preventive and therapeutic effects on bleomycin-induced skin fibrostic disease.

### Example B4: Preventive and Therapeutic Effects of Compound I-1 on Pulmonary Fibrosis

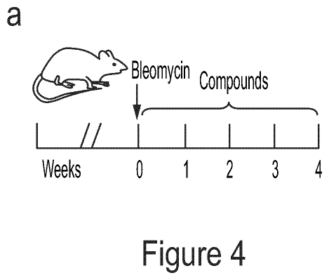

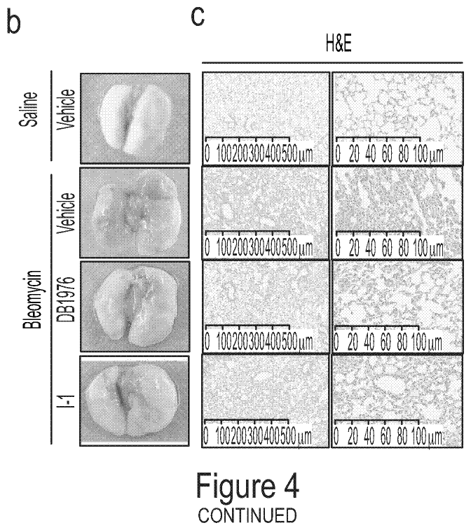

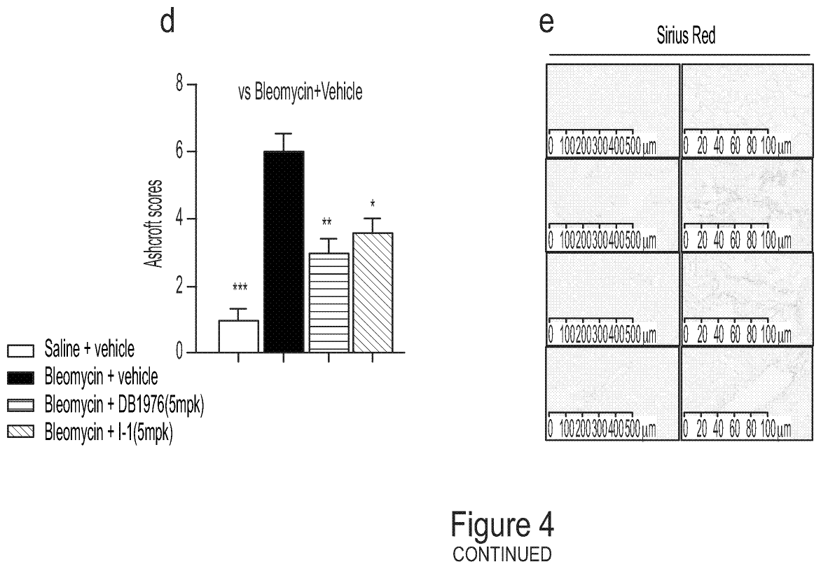

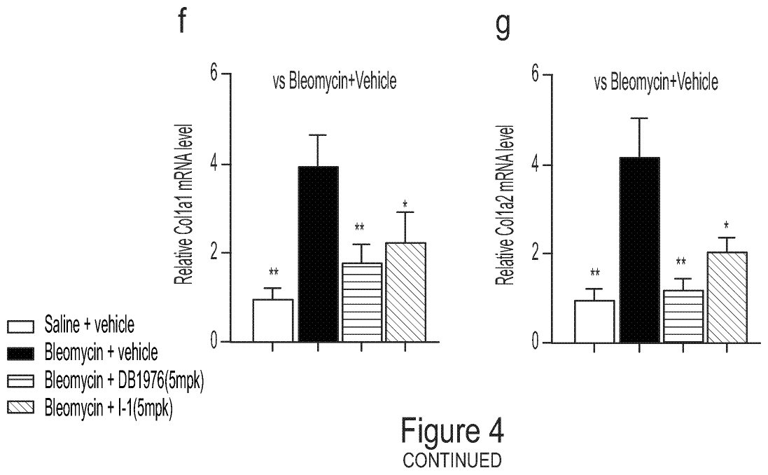

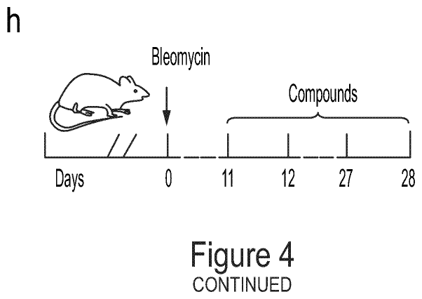

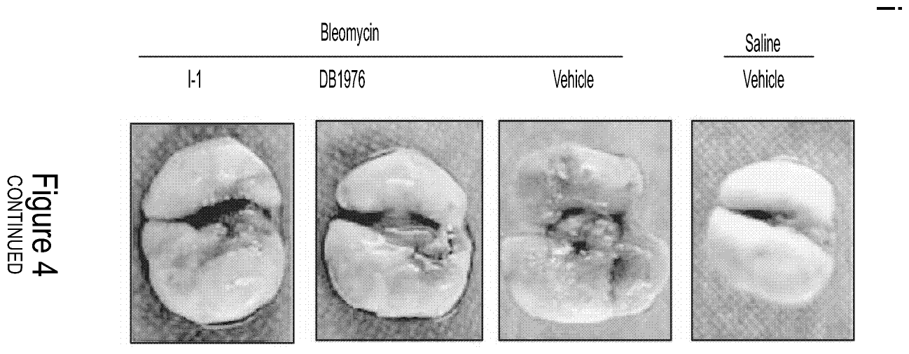

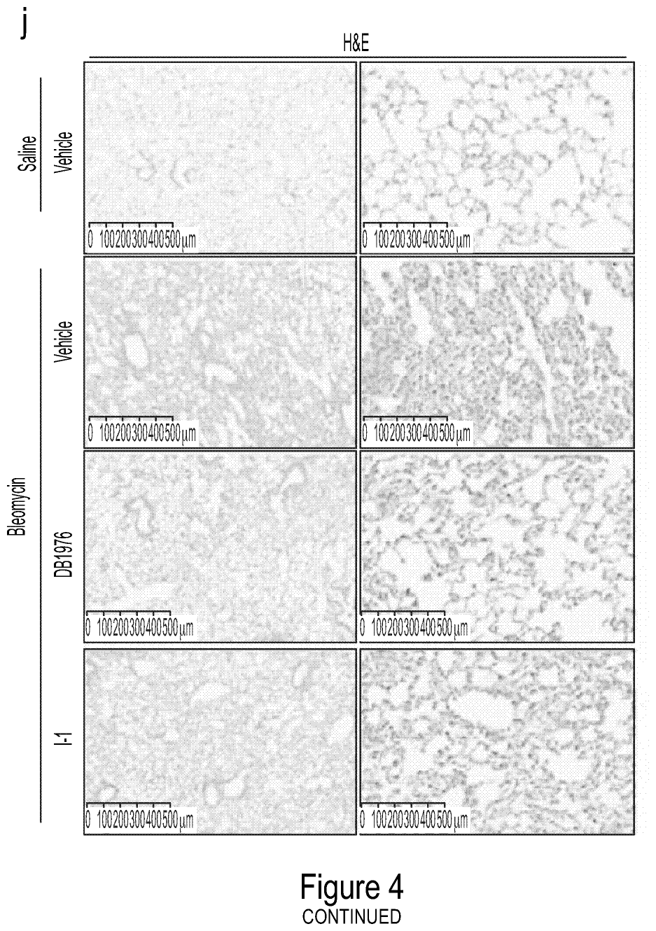

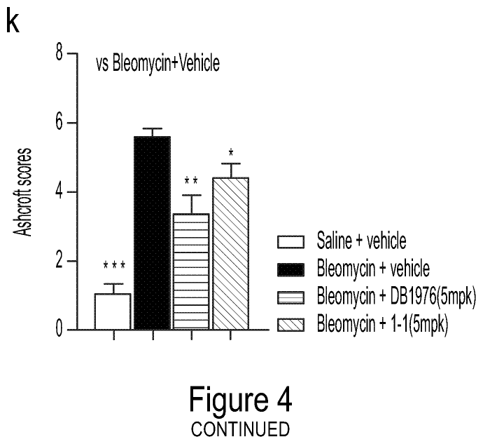

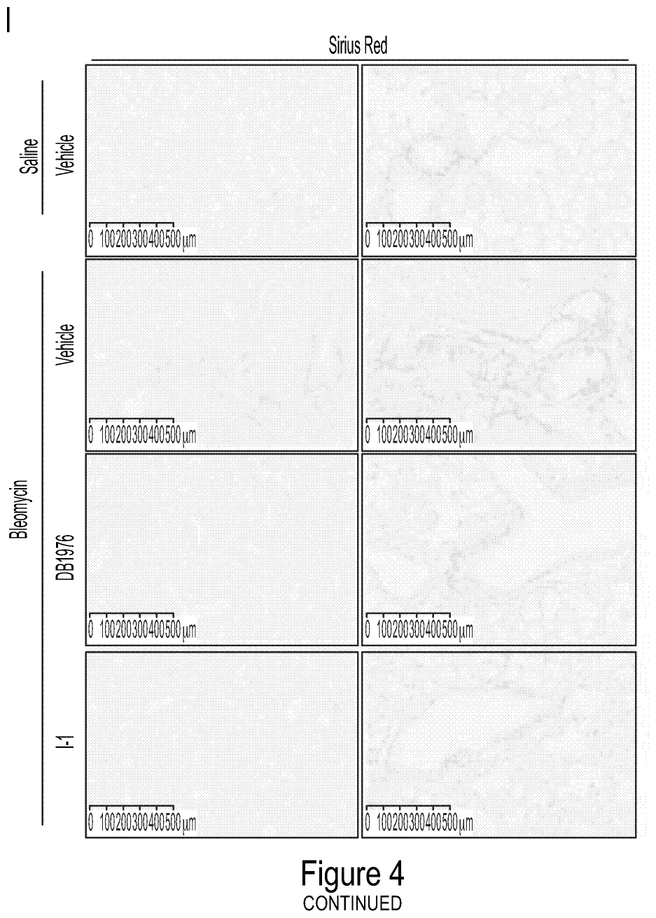

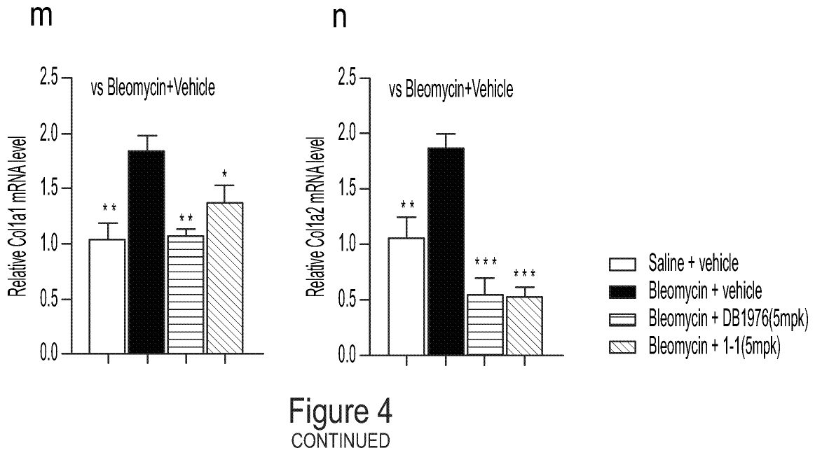

Methods: To evaluate the preventive and therapeutic effects of compound I-1 on pulmonary fibrosis, bleomycin was used to establish two animal models of pulmonary fibrosis with different drug intervention (6-8 weeks, C₅₇BL/6, male). Bleomycin (0.025U, Code.D11063, OKA, China) was injected by a single intratracheal application. Equal volumes of sterilized saline served as a control. (I) Preventive model of bleomycin-induced pulmonary fibrosis. Compounds compound I-1, positive control DB1976, or vehicle (saline) were treated (injected intraperitoneally, i.p.) immediately after single bleomycin injection for 4 weeks (FIG. 4a). (II) Therapeutic model of bleomycin-induced pulmonary fibrosis. Mice were pre-charged with bleomycin for 11 days to induced pulmonary fibrosis, then treated with compounds compound I-1, positive control DB1976, or vehicle (saline) for 17 days, total time is 4 weeks after bleomycin treatment (FIG. 4h). After the last day of treatment of both models, mice were fasted overnight and euthanized. A part of lung was fixed with paraformaldehyde, embedded with paraffin, and then sliced for H&E and Sirius red staining to check pathological features, and Ashcroft scores. Hubner, R. H. et al. Biotechniques 44, 507-511, 514-507, doi:10.2144/000112729 (2008). Another part of lung were collected and stored in −80° C. after liquid nitrogen quick-freezing, RNA isolation (Code.R6934, OMEGA, USA), first cDNA reverse (Code.AT341, TransGen, China), SYBR mix (Code.AQ601, TransGen, China) for Q-PCR (LightCycle96, Roche) to verify several fibrosis-related genes mRNA level, such as, Col1a1 and Col1a2.

Results: As shown in FIG. 4, bleomycin treatment for 4 weeks significantly induced pulmonary fibrosis pathological features, including lung deterioration, collagen deposition, alveolar wall thickening and alveolar structure destruction when compared with saline/vehicle group, which indicated the successful establishment of the bleomycin-induced pulmonary fibrosis model. Comparing with the bleomycin/vehicle group, compound I-1 and DB1976 treatment significantly prevented the pulmonary fibrosis progress, as measured by pathological changes based on staining and Ashcroft scores, collagen deposition indicated by Sirius red staining, and Col1a1, Col1a2 mRNA levels (FIG. 4a-4g). In the therapeutic model, treatment with compound I-1 reversed and impede bleomycin-induced pulmonary fibrosis, including the above-mentioned pathological features (FIG. 4h-4n). These data suggest that compound I-1 has preventive and therapeutic effects on bleomycin-induced pulmonary fibrosis disease.

### Example B5: Therapeutic Effects of Compound I-1 on NASH and Liver Fibrosis

Methods: To evaluate the potential therapeutic effects of compound I-1 on liver disorders, including liver fat steatosis and accumulation, nonalcoholic fatty liver disease (NAFLD), nonalcoholic steatosis hepatitis (NASH), and liver fibrosis, three mice models (C₅₇BL/6, male, 8 weeks old) were used. (I) NASH diet (Code.TD.160785, ENVIGO, USA)-induced NASH model: All mice were fed with NASH diet for 10 weeks. Mice were then randomly distributed into three groups, and each group was assigned to be injected daily with vehicle, compound I-1 (2.5mpk or 5mpk, i.p.), or DB1976 (2.5mpk, i.p, as a positive control) for another 6 weeks on continuous NASH diet. A group with normal regular diet was treated with vehicle as health control. (II) High-fat diet (HFD) combinate with CCl₄ (lower dose)—induced NASH model. Mice were fed with normal regular diet or HFD (kcal fat 60%—D12492, research diets) for 10 weeks, then the HFD mice were divided into two groups randomly based on the rule of minimum weight differences. Each group was assigned to be injected with CCl₄ (25% v/v in olive oil, 0.5 mL/kg body weight) or pure olive oil (i.p.) twice a week for 4 weeks. Compound 1-1 or vehicle (saline) were injected (i.p.) once daily for 4 weeks at the same time with CCl₄ under continuous HFD. (III) CCl₄ (higher dose)—induced liver fibrosis model. Liver fibrosis was induced by CCl₄ injection (20% v/v in olive oil, 10 mL/kg body weight) twice a week for 6 weeks, compound I-1 or vehicle are also injected (i.p.) once daily for 6 weeks at the same time with CCl₄ application. Ail the mice in the above-mentioned three models were observed daily. After the last day of treatment of all the models, mice were fasted overnight and euthanized. Serum was collected after centrifuging of the whole blood at 4 degrees, and blood biochemistry parameters were tested through automatic biochemical instrument (BS-240VET, Mindray). Part of liver tissues were fixed with paraformaldehyde, embedded with paraffin, and then sliced for H&E or Sirius red staining to check pathological features, and NAFLD scores. Kleiner, D. E. et al. Hepatology 41, 1313-1321, doi:10.1002/hep.20701 (2005). Part of liver tissues were used fresh, embedded in optimum cutting temperature compound, and sectioned. The sections were stained with 0.5% oil red 0 according to standard procedures after fixed in 4% paraformaldehyde in PBS. Another part of liver tissues were collected and stored in −80° C. after liquid nitrogen quick-freezing, RNA. isolation (Code.R6934, OMEGA., USA), first cDNA reverse (Code.AT341, TransGen, China), SYBR mix (Code.AQ601, TransGen, China) for Q-PCR (LightCycler®96, Roche) to verify genes mRNA level, such as, fibrosis-related genes, Col1a1 and Col1a2; inflammation-related genes, IL-6 and IL-1β.

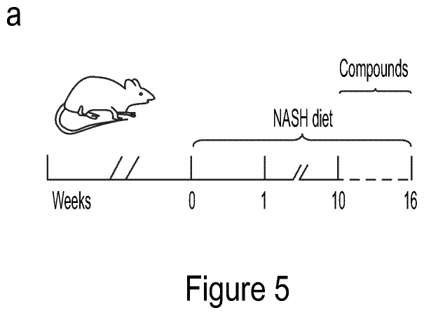

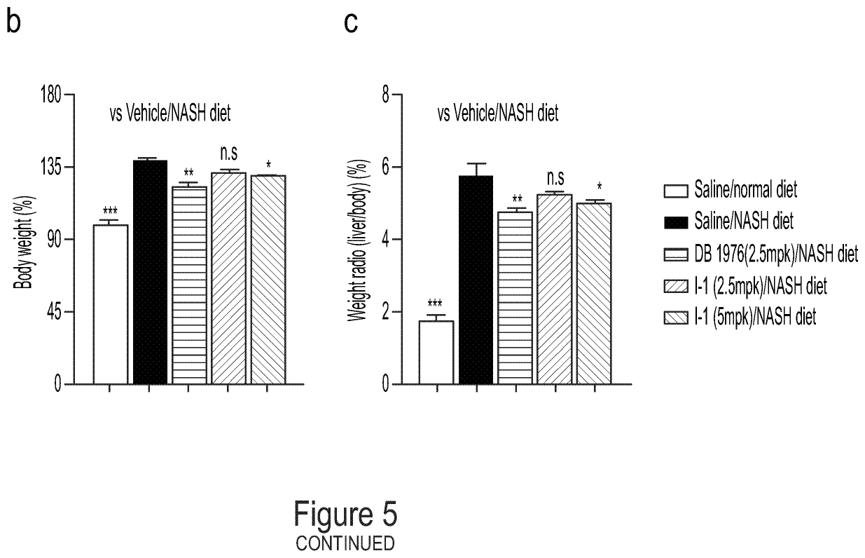

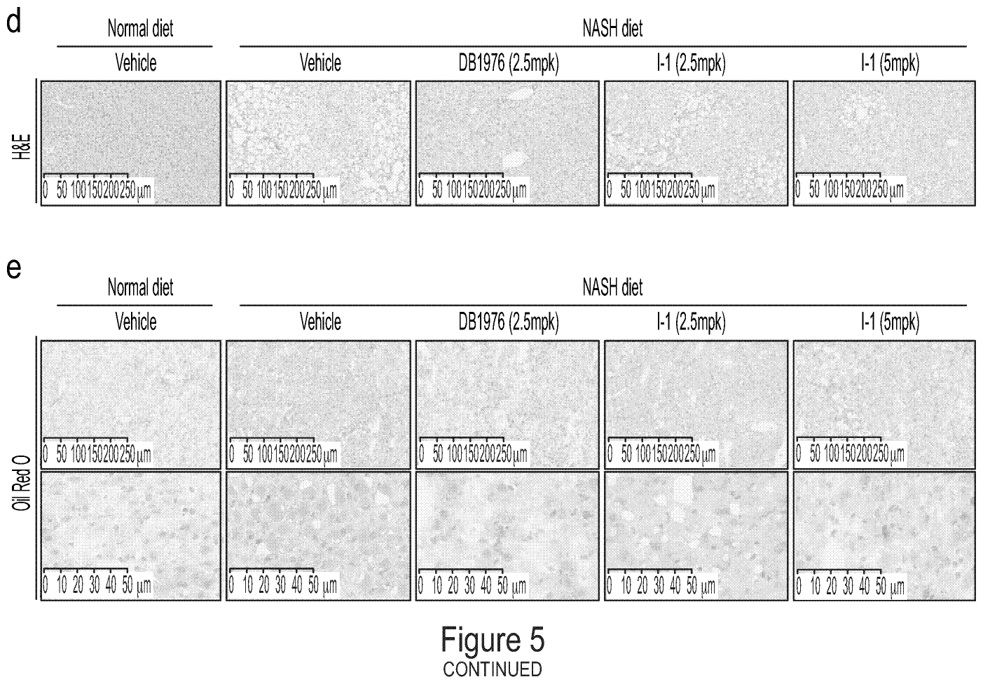

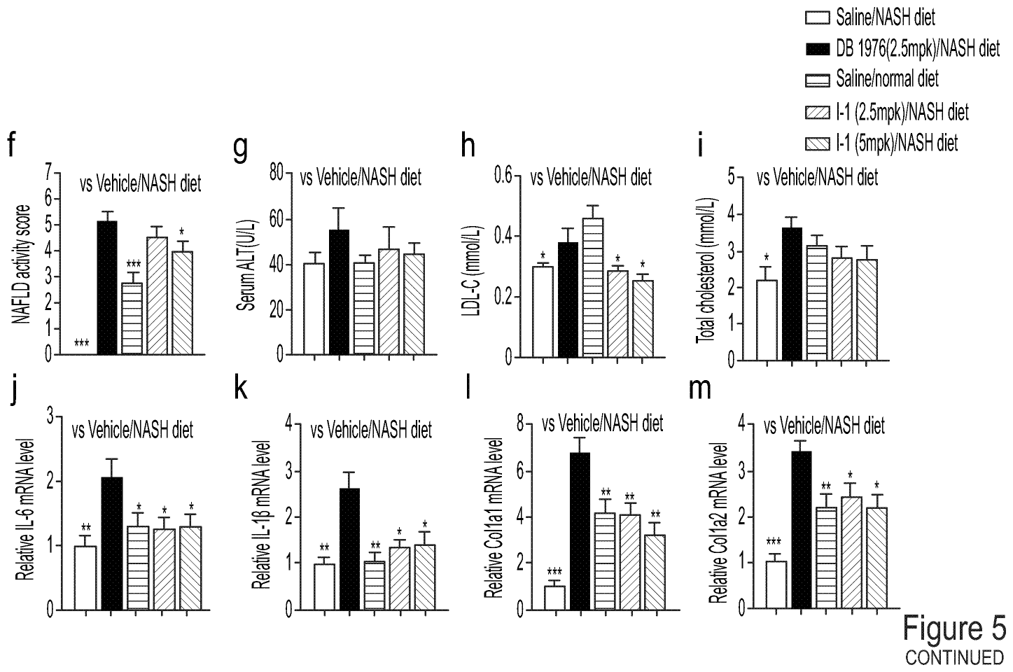

Result 1: NASH diet—induced NASH model. As shown in FIG. 5, NASH diet for 16 weeks significantly increased body weight and liver/body radio (FIG. 5b-5c); induced large fat accumulation in liver, including larger and more lipid droplets based on pathological staining (FIG. 5d-5f), besides, NASH diet application raised not only serum parameters, such as, ALT, LDL-C and total cholesterol (TC) (FIG. 5g-5i), but inflammation and fibrosis-related genes, IL-6, IL-1β and Col1a1, Col1a2 (FIG. 5j-5m). Treatment with compound I-1 (5mpk) and DB1976 (2.5mpk) for 6 weeks potently alleviated the above-mentioned metabolic disturbance by NASH diet. Compound I-1 (2.5mpk) showed lower efficacy when comparing with DB1976 (2.5mpk), but also showed a tendency to alleviate metabolic disorders as evidenced by decreased fat accumulation in liver and lower mRNA levels of IL-6, IL-1β and Col1a1, Col1a2. These data suggest that compound I-1 has therapeutic potential for treatment of liver fat accumulation, inflammation, and NASH.

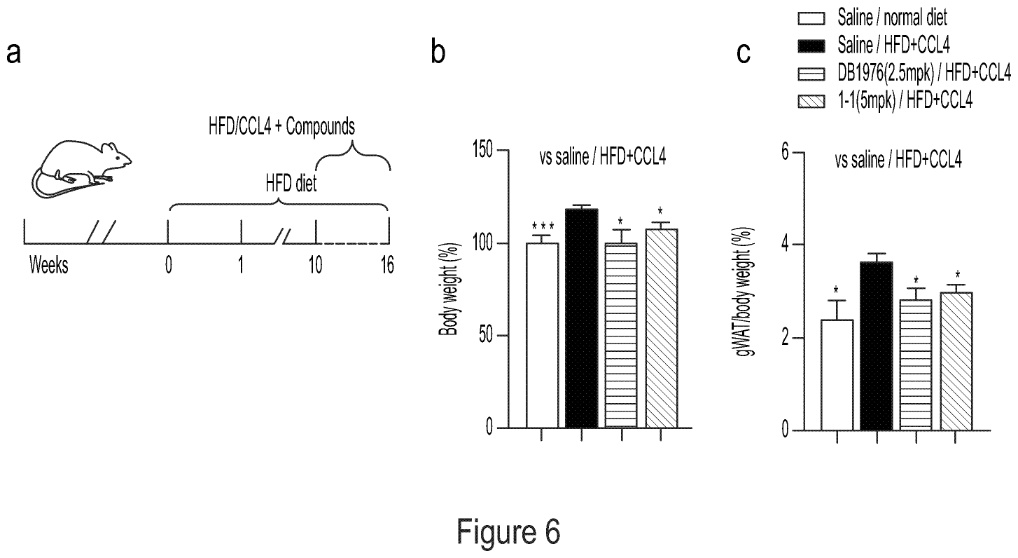

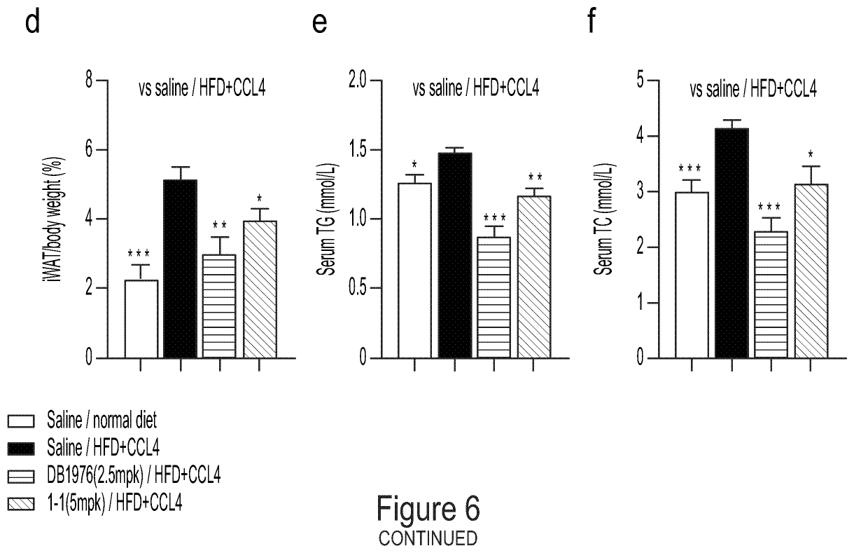

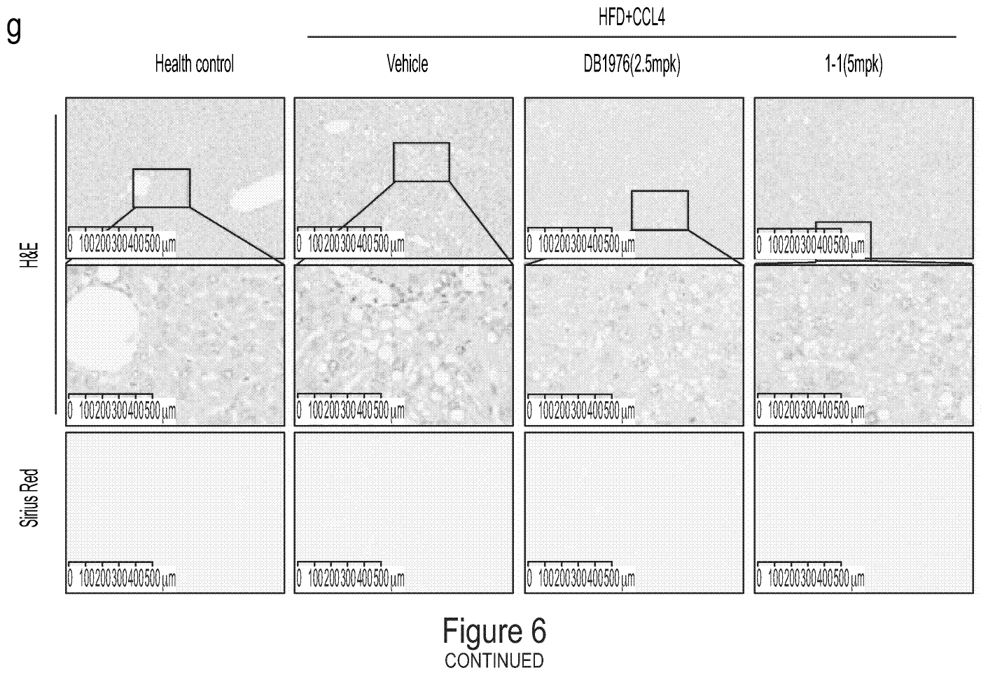

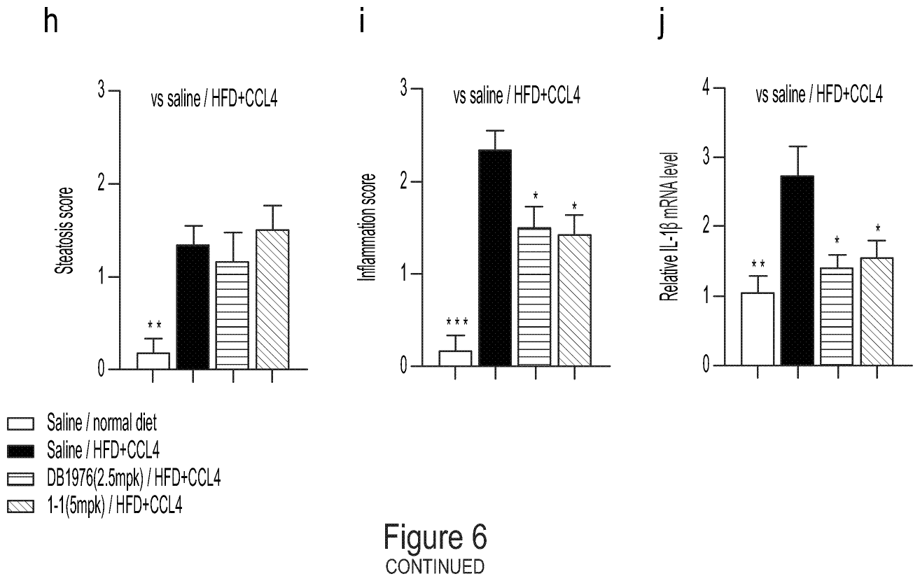

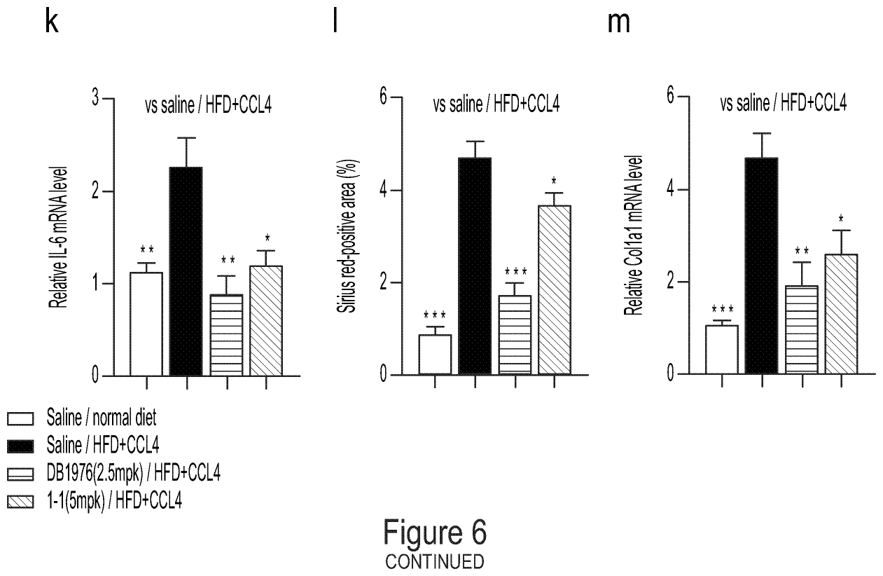

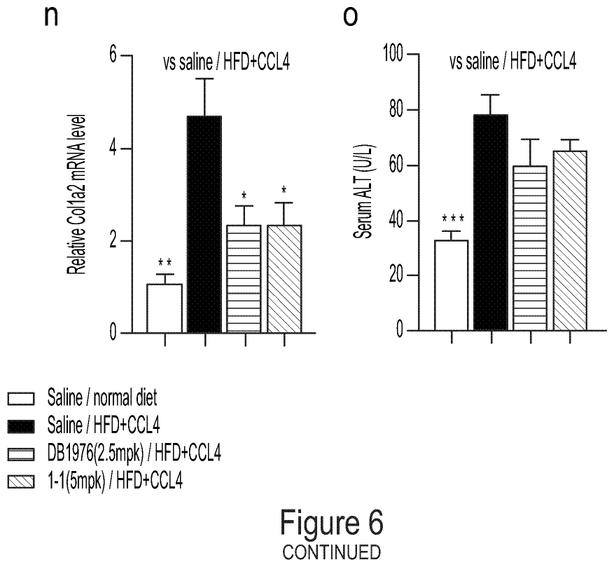

Result 2: High-fat diet (HFD) combinate with CCl₄ (lower dose)—induced NASH model. AS shown in FIG. 6, pre-treated by HFD and following 6 weeks CCL4 treatment significantly increased body weight, gonadal white adipose tissue (gWAT) and inguinal white adipose tissue (iWAT) weight radio, treatment with DB1976 and 1-1 reversed them (FIG. 6b-6d). HFD/CCL4 treatment induced dyslipidemia in mice, the application of DB1976 and I-1 reduced serum triglyceride (TG) and total-cholesterol (TC) (FIG. 6e-6f). Additionally, histological examination with H&E staining showed that I-1 is not efficacious at reducing fat accumulation (FIG. 6g), is consistent with liver steatosis score (FIG. 6h), but is efficacious for reducing inflammatory response, indicated by reduced inflammatory infiltrates (FIG. 6g), inflammation score (FIG. 6i) and liver mRNA level of IL-113 (FIG. 6j) and IL-6 (FIG. 6k). Notably, administration with DB1976 and I-1 significantly alleviated liver fibrosis induced by HFD/CCL4 application, reduced collagen deposition indicated by sirius red staining (FIGS. 6g and 6l) and liver mRNA level of Col1a1 (FIG. 6m) and Col1a2 (FIG. 6n). Meanwhile, I-1 treatment showed the tendency to reduce the level of serum ALT (FIG. 6o), which suggest 1-1 is not hepatotoxic under its effective concentration. These data suggest that compound I-1 shows anti-inflammation and anti-fibrosis potential in HFD/CCL4-induced NASH and liver fibrosis mice.

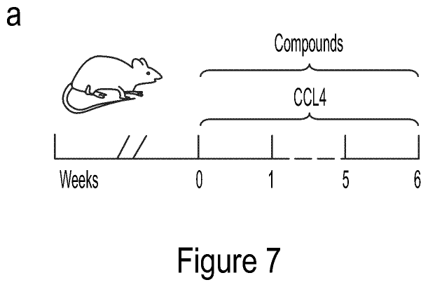

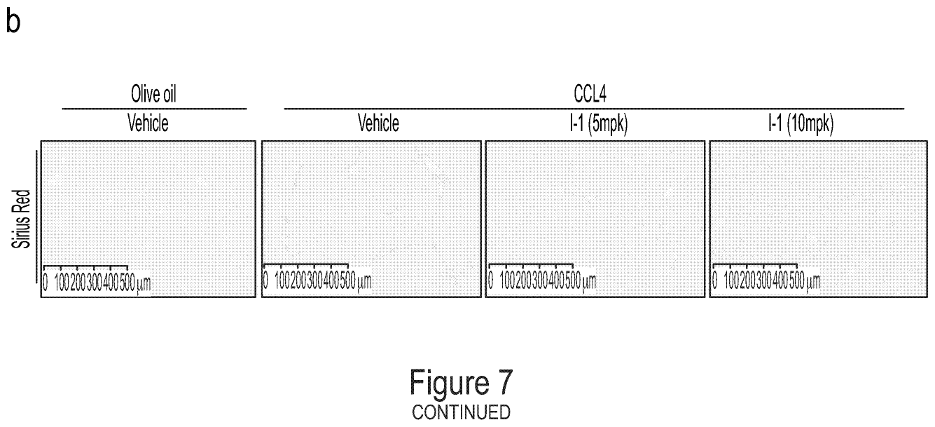

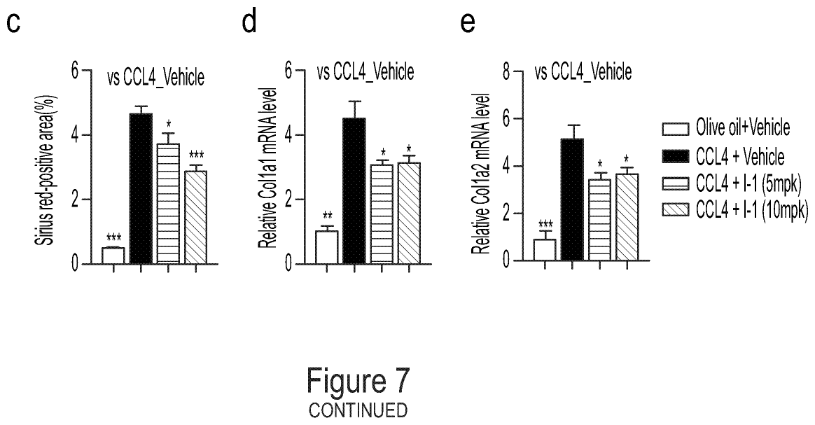

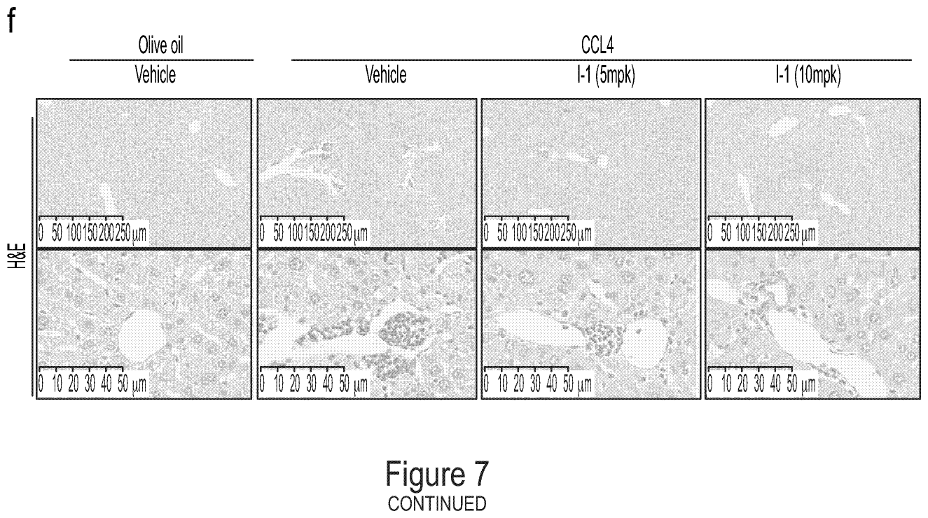

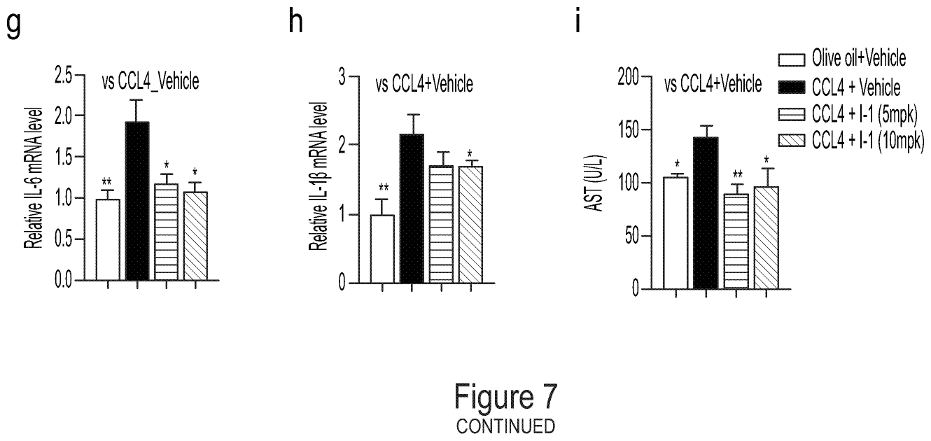

Result 3: CCl₄ (higher dose)—induced liver fibrosis. As shown in FIG. 7, CCL4 treatment for 6 weeks significantly induced robust CCl₄-induced liver fibrosis parameters such as large collagen deposition, high fibrosis degree (FIGS. 7b-7c and 7f) and inflammatory response based on Sirius red and H&E staining. CCl₄ application also significantly increased inflammation- and fibrosis-related genes, IL-6, IL-1β (FIG. 7g-7h) and Col1a1, Col1a2 (FIG. 7d-7e), but AST level in serum (FIG. 7i). Treatment with compound I-1 (5mpk or 10mpk) alleviated CCl₄-induced liver fibrosis, significantly decreased the collagen deposition and Sirius red-positive area, and ameliorated the inordinate mRNA level and blood biochemistry induced by CCl₄ application. These data suggest that compound I-1 has preventive and therapeutic potential for liver fibrosis.

All publications, including patents, patent applications, and scientific articles, mentioned in this specification are herein incorporated by reference in their entirety for all purposes to the same extent as if each individual publication, including patent, patent application, or scientific article, were specifically and individually indicated to be incorporated by reference.

1. A compound of formula (I), or a stereoisomer or a pharmaceutically acceptable salt thereof, 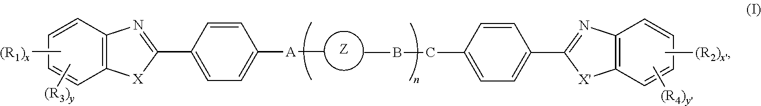
wherein:
x and x′ are each independently 0, 1, 2, 3, or 4;
each of R₁ and R₂ is independently —Rₐ, —N(Rₐ)₂, —ORₐ, —C(O)ORₐ, —OC(O)Rₐ, —NHC(O)Rₐ, —C(O)N(Rₐ)₂, —OC(O)N(Rₐ)₂, —NHC(O)N(Rₐ)₂, —S(O)₂Rₐ, —S(O)₂N(Rₐ)₂, —C(O)Rₐ, —NHS(O)₂Rₐ, —NHS(O)₂N(Rₐ)₂, nitro, cyano, or halogen, wherein each Rₐ is independently hydrogen, C₁₋₆ alkyl, C₃₋₈ cycloalkyl, 3-12 membered heterocyclyl, C₆₋₁₂ aryl, or 5-12 membered heteroaryl, and wherein any two of R₁ or any two of R₂ can be taken together with the atoms to which they attach to form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
y and y′ are each independently 0, 1, 2, 3, or 4;
R₃ is 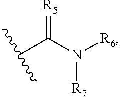
wherein
R₅ is O, S, or NH, and
R₆ and R₇ are each independently hydrogen, C₁₋₆ alkyl, C₃₋₈ cycloalkyl, 3-12 membered heterocyclyl, C₆₋₁₂ aryl, 5-12 membered heteroaryl, —C(O)ORd or —S(O)₂R_{d}, wherein each R_{d} is independently hydrogen, C₁₋₁₂ alkyl, C₃₋₈ cycloalkyl, 3-12 membered heterocyclyl, C₆₋₁₂ aryl, or 5-12 membered heteroaryl, and wherein R₆ and R₇ can be taken together with the nitrogen atom to which they attach to form a 3-12 membered heterocyclyl or 5-12 membered heteroaryl, or
when y is 2, 3, or 4, then two R₃ can be taken together with the atoms to which they attach to form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
R₄ is 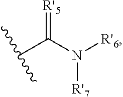
wherein
R′₅ is O, S, or NH, and
R′₆ and R′₇ are each independently hydrogen, C₁₋₆ alkyl, C₃₋₈ cycloalkyl, 3-12 membered heterocyclyl, C₆₋₁₂ aryl, 5-12 membered heteroaryl, —C(O)OR_{d} or —S(O)₂R_{d}, wherein each R_{d} is independently hydrogen, C₁₋₁₂ alkyl, C₃₋₈ cycloalkyl, 3-12 membered heterocyclyl, C₆₋₁₂ aryl, or 5-12 membered heteroaryl, and wherein R′₆ and R′₇ can be taken together with the nitrogen atom to which they attach to form a 3-12 membered heterocyclyl or 5-12 membered heteroaryl, or
when y′ is 2, 3, or 4, then two R₄ can be taken together with the atoms to which they attach for form a C₃₋₈ cycloalkyl, 3-12 membered heterocyclyl, C₆₋₁₂ aryl, or 5-12 membered heteroaryl, wherein each of the C₃₋₈ cycloalkyl, 3-12 membered heterocyclyl, C₆₋₁₂ aryl, and 5-12 membered heteroaryl is independently optionally substituted by R₉;
X is O, S, NH, or NR₈ and X′ is O, S, NH, or NR's, wherein
R₈ and R′₈ are each independently C₁₋₆ alkyl, C₃₋₈ cycloalkyl, 3-12 membered heterocyclyl, C₆₋₁₂ aryl, or 5-12 membered heteroaryl;
A and B are each independently —C(O)—, —C(O)NH—, —NHC(O)—, —S(O)₂—, —S(O)₂NH—, or —NHS(O)₂—;
C is a chemical bond or —NH—, provided that
when B is —C(O)— or —S(O)₂—, then C is —NH—, and
when B is —C(O)NH—, —NHC(O)—, —S(O)₂NH—, or —NHS(O)₂—, then C is a chemical bond;
n is an integer selected from 1-6;
each Z is independently C₁₋₆ alkyl, C₃₋₈ cycloalkyl, 3-12 membered heterocyclyl, C₆₋₁₂ aryl, or 5-12 membered heteroaryl, each of which is independently optionally substituted by Rₑ, wherein each Rₑ is independently C₁₋₆ alkyl, C₁₋₆ alkoxyl, C₃₋₈ cycloalkyl, 3-12 membered heterocyclyl, C₆₋₁₂ aryl, 5-12 membered heteroaryl, amino, hydroxyl, carboxyl, nitro, cyano, or halogen,
provided that at least one Z is C₃₋₈ cycloalkyl, 3-12 membered heterocyclyl, C₆₋₁₂ aryl, or 5-12 membered heteroaryl, each of which is independently optionally substituted by Rₑ; and
each R₉ is independently —R_{b}, —N(R_{b})₂, —OR_{b}, —C(O)OR_{b}, —OC(O)R_{b}, —NHC(O)R_{b}, —C(O)N(R_{b})₂, —OC(O)N(R_{b})₂, —NHC(O)N(R_{b})₂, —S(O)₂R_{b}, —S(O)₂N(R_{b})₂, —C(O)R_{b}, —NHS(O)₂R_{b}, —NHS(O)₂N(R_{b})₂, nitro, cyano, or halogen, wherein each R_{b} is independently hydrogen, C₁₋₆ alkyl, C₃₋₈ cycloalkyl, 3-12 membered heterocyclyl, C₆₋₁₂ aryl, or 5-12 membered heteroaryl.

2. The compound of claim 1, or a stereoisomer or a pharmaceutically acceptable salt thereof, wherein x and x′ are each independently 2 or 3.

3. The compound of claim 1, or a stereoisomer or a pharmaceutically acceptable salt thereof, wherein each of R₁ and R₂ is independently hydrogen, methyl, methoxyl, or fluoro.

4. The compound of claim 1, or a stereoisomer or a pharmaceutically acceptable salt thereof, wherein each of R₁ and R₂ is hydrogen.

5. The compound of claim 1, or a stereoisomer or a pharmaceutically acceptable salt thereof, wherein y and y′ are each independently 1 or 2.

6. The compound of claim 1, or a stereoisomer or a pharmaceutically acceptable salt thereof, wherein R₅ is O or NH; and R₆ and R₇ are each independently hydrogen or —C(O)OR_{d}.

7. The compound of claim 1, or a stereoisomer or a pharmaceutically acceptable salt thereof, wherein R₅ is NH; and each of R₆ and R₇ is hydrogen.

8. The compound of claim 1, or a stereoisomer or a pharmaceutically acceptable salt thereof, wherein R′₅ is O or NH; and R′₆ and R′₇ are each independently hydrogen or —C(O)OR_{d}.

9. The compound of claim 1, or a stereoisomer or a pharmaceutically acceptable salt thereof, R′₅ is NH; and each of R′₆ and R′₇ is hydrogen.

10. The compound of claim 1, or a stereoisomer or a pharmaceutically acceptable salt thereof, wherein two R₃ and/or two R₄ are taken together with the atoms to which they attach to form a 5-12 membered heteroaryl, which is optionally substituted by R₉.

11. The compound of claim 10, or a stereoisomer or a pharmaceutically acceptable salt thereof, wherein two R₃ and/or two R₄ are taken together with the atoms to which they attach to form

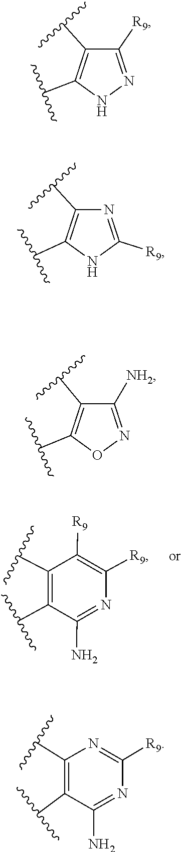

12. The compound of claim 11, or a stereoisomer or a pharmaceutically acceptable salt thereof, wherein two R₃ and/or two R₄ are taken together with the atoms to which they attach to form

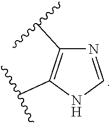 [SMILES: *c1nc[nH]c1*]

13. The compound of claim 1, or a stereoisomer or a pharmaceutically acceptable salt thereof, wherein X is NH or NR₈ and X′ is NH or NR′₈, wherein R₈ and R′₈ are each independently C₁₋₆ alkyl.

14. The compound of claim 13, or a stereoisomer or a pharmaceutically acceptable salt thereof, wherein X and X′ are both NH.

15. The compound of claim 1, or a stereoisomer or a pharmaceutically acceptable salt thereof, wherein A and B are each independently —C(O)—, —C(O)NH—, or —NHC(O)—.

16. The compound of claim 15, or a stereoisomer or a pharmaceutically acceptable salt thereof, wherein each of A and B is —C(O)—.

17. The compound of claim 1, or a stereoisomer or a pharmaceutically acceptable salt thereof, wherein n is 2.

18. The compound of claim 1, or a stereoisomer or a pharmaceutically acceptable salt thereof, wherein each Z is independently C₁₋₆ alkyl, 3-12 membered heterocyclyl, or 5-12 membered heteroaryl, each of which is independently optionally substituted by Rₑ.

19. The compound of claim 1, or a stereoisomer or a pharmaceutically acceptable salt thereof, wherein each Z is independently —CH₂—, —CH₂CH₂—, 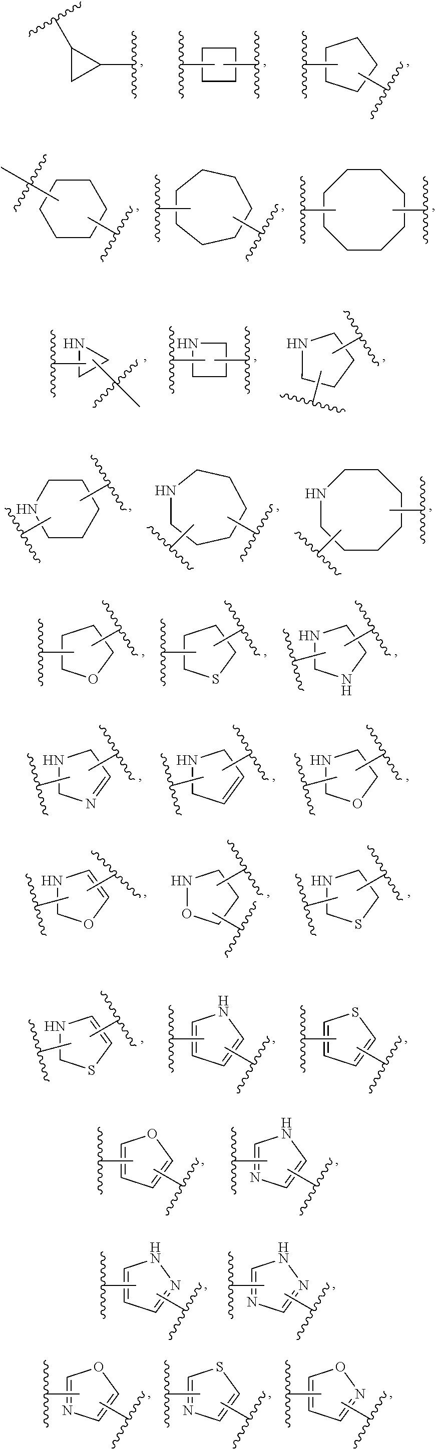 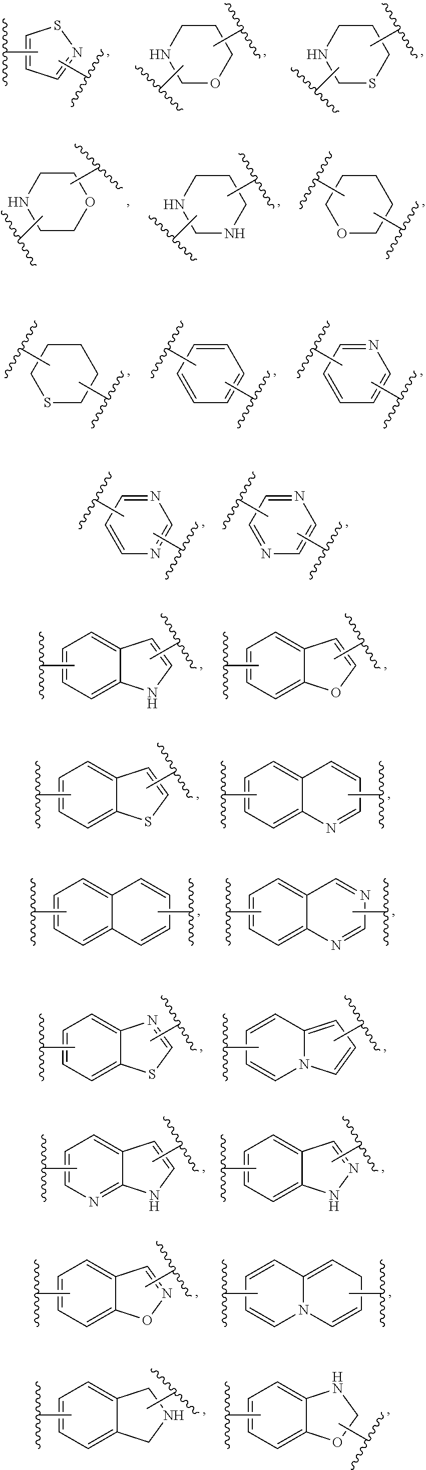 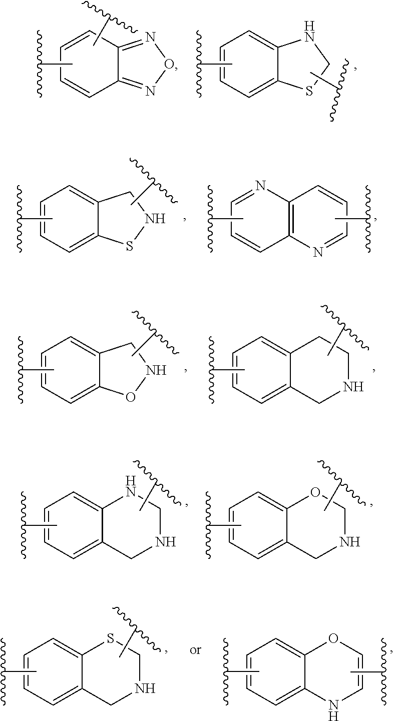
each of which is independently optionally substituted by Rₑ.

20. The compound of claim 1, or a stereoisomer or a pharmaceutically acceptable salt thereof, wherein Z is

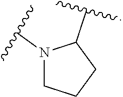 [SMILES: *C1CCCN1*]

21. The compound of claim 1, or a pharmaceutically acceptable salt thereof, wherein the compound is selected from the group consisting of:

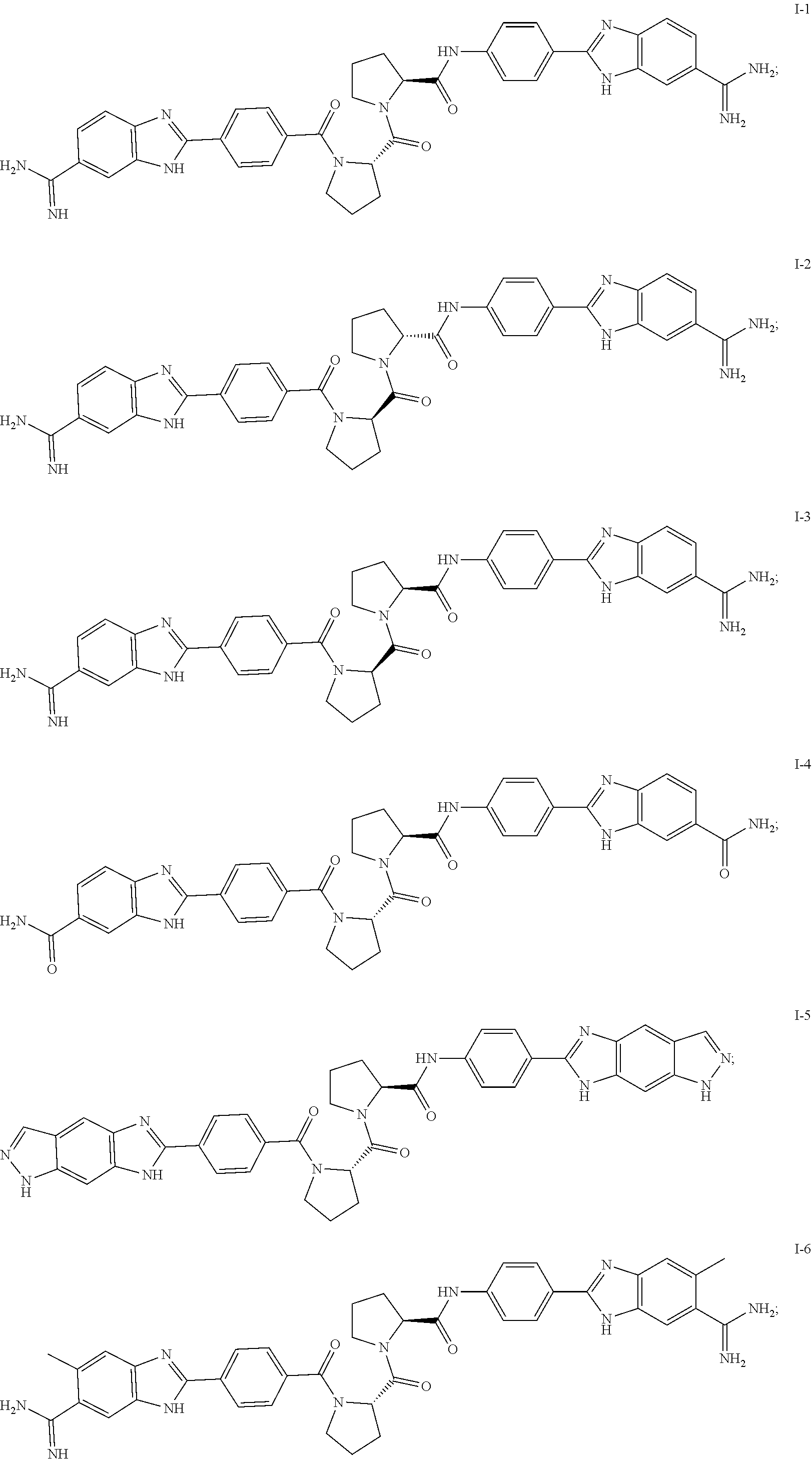

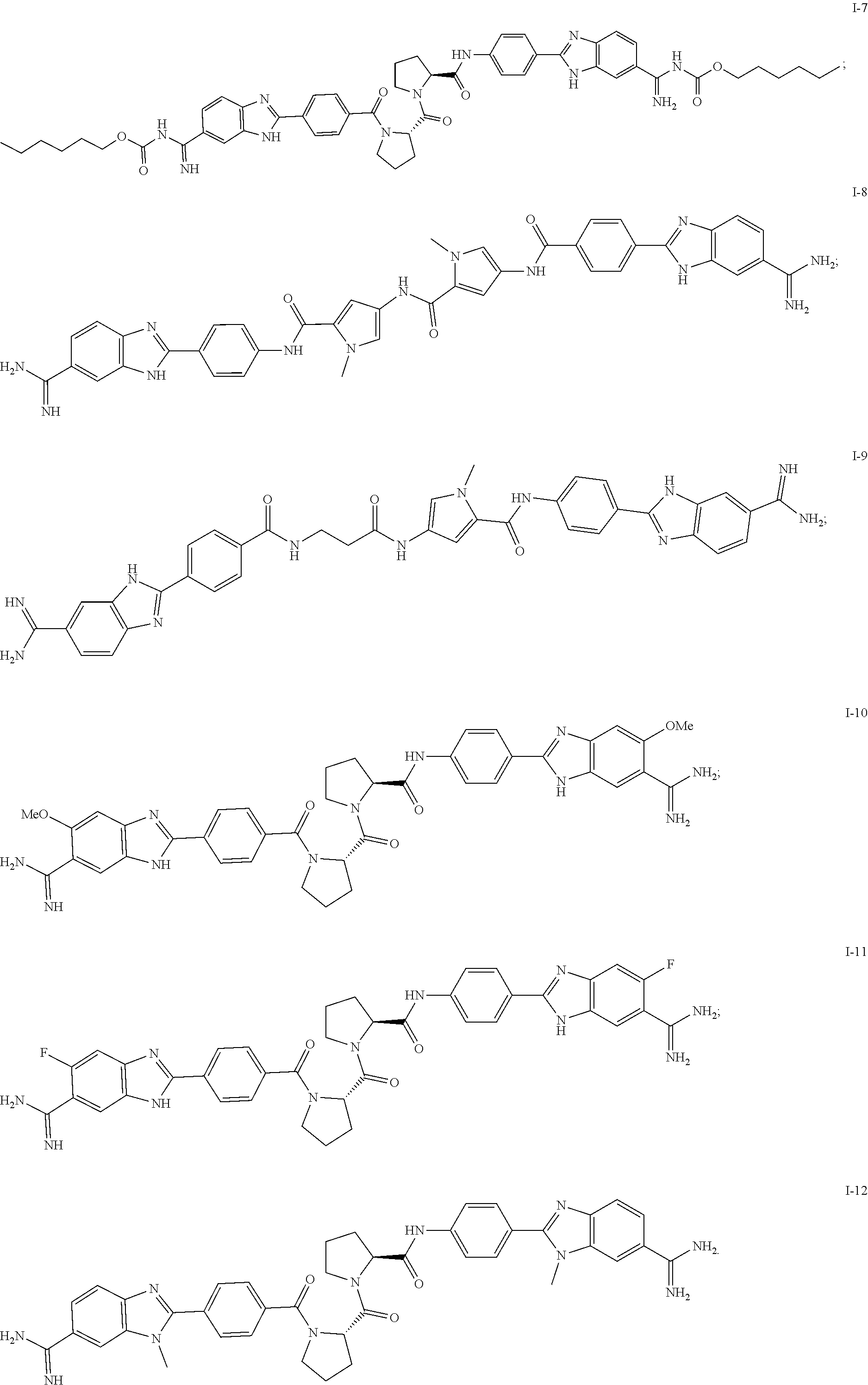

22. A method of preparing the compound of claim 1, or a stereoisomer or a pharmaceutically acceptable salt thereof, comprising converting a compound of formula (II): 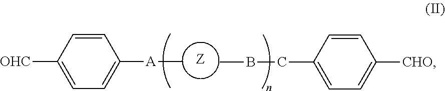
or a stereoisomer or a pharmaceutically acceptable salt thereof, to the compound of formula (I),
or a stereoisomer or a pharmaceutically acceptable salt thereof.

23. The method of claim 22, wherein the compound of formula (II) is of formula (13′) 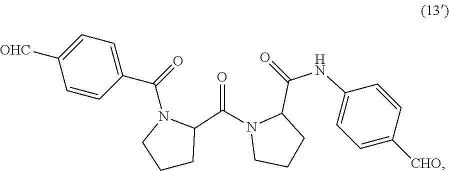 [SMILES: O=Cc1ccc(NC(=O)C2CCCN2C(=O)C2CCCN2C(=O)c2ccc(C=O)cc2)cc1]
or a stereoisomer or a pharmaceutically acceptable salt thereof, and the method further comprises:
(a) reacting a compound of formula (11′), 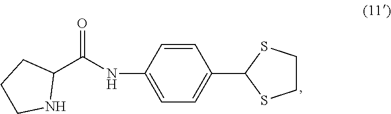 [SMILES: O=C(Nc1ccc(C2SCCS2)cc1)C1CCCN1]
or a stereoisomer or a pharmaceutically acceptable salt thereof, with a compound of formula (5′), 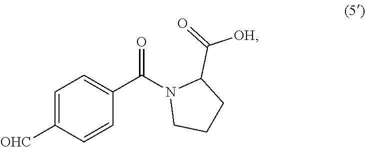 [SMILES: O=Cc1ccc(C(=O)N2CCCC2C(=O)O)cc1]
or a stereoisomer or a pharmaceutically acceptable salt thereof;
(b) converting a compound of formula (6), 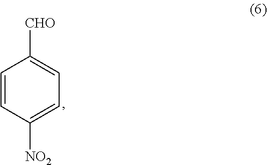 [SMILES: O=Cc1ccc([N+](=O)[O-])cc1]
or a stereoisomer or a pharmaceutically acceptable salt thereof, to the compound of formula (11′), or a stereoisomer or a pharmaceutically acceptable salt thereof; and/or
(c) converting a compound of formula (1), 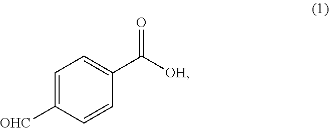 [SMILES: O=Cc1ccc(C(=O)O)cc1]
or a stereoisomer or a pharmaceutically acceptable salt thereof, to the compound of formula (5′),
or a stereoisomer or a pharmaceutically acceptable salt thereof.

24. The compound of claim 22, wherein the compound of formula (II) is of formula (50), 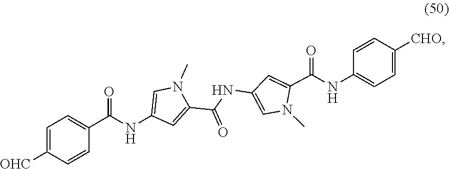 [SMILES: Cn1cc(NC(=O)c2ccc(C=O)cc2)cc1C(=O)Nc1cc(C(=O)Nc2ccc(C=O)cc2)n(C)c1]
or a stereoisomer or a pharmaceutically acceptable salt thereof, and the method further comprises:
(a) reacting a compound of formula (45), 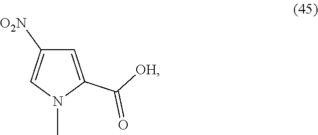
or a stereoisomer or a pharmaceutically acceptable salt thereof, with a compound of formula (41), 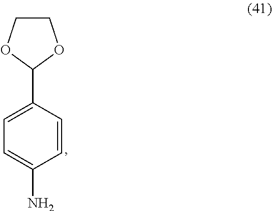 [SMILES: Nc1ccc(C2OCCO2)cc1]
or a stereoisomer or a pharmaceutically acceptable salt thereof;
(b) converting a compound formula (42) 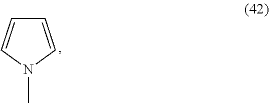
or a stereoisomer or a pharmaceutically acceptable salt thereof, to the compound of formula (45),
or a stereoisomer or a pharmaceutically acceptable salt thereof; and/or
(c) converting a compound of formula (6), 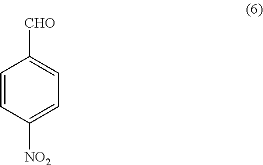 [SMILES: O=Cc1ccc([N+](=O)[O-])cc1]
or a stereoisomer or a pharmaceutically acceptable salt thereof, to the compound of formula (41),
or a stereoisomer or a pharmaceutically acceptable salt thereof.

25. The method of claim 22, wherein the compound of formula (II) is of formula (54), 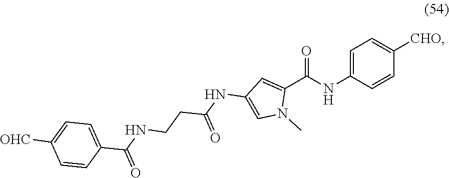 [SMILES: Cn1cc(NC(=O)CCNC(=O)c2ccc(C=O)cc2)cc1C(=O)Nc1ccc(C=O)cc1]
or a stereoisomer or a pharmaceutically acceptable salt thereof, and the method further comprises:
(a) converting a compound of formula (53), 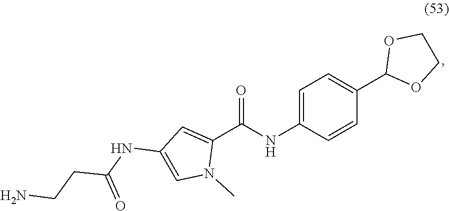 [SMILES: Cn1cc(NC(=O)CCN)cc1C(=O)Nc1ccc(C2OCCO2)cc1]
or a stereoisomer or a pharmaceutically acceptable salt thereof, to the compound of formula (54),
or a stereoisomer or a pharmaceutically acceptable salt thereof;
(b) converting a compound of formula (47), 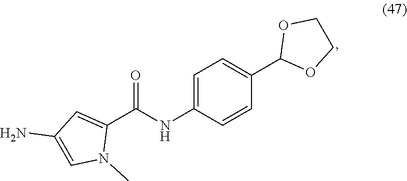 [SMILES: Cn1cc(N)cc1C(=O)Nc1ccc(C2OCCO2)cc1]
or a stereoisomer or a pharmaceutically acceptable salt thereof, to the compound of formula (53),
or a stereoisomer or a pharmaceutically acceptable salt thereof; and/or
(c) converting a compound of formula (45), 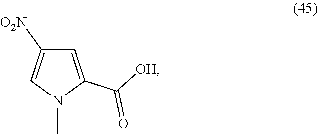
or a stereoisomer or a pharmaceutically acceptable salt thereof, to the compound of formula (47),
or a stereoisomer or a pharmaceutically acceptable salt thereof.

26. A pharmaceutical composition comprising the compound of claim 1, or a stereoisomer or a pharmaceutically acceptable salt thereof, and a pharmaceutically acceptable carrier or excipient.

27. (canceled)

28. A method of treating a PU.1-mediated disease in an individual in need thereof, comprising administering an effective amount of the compound of claim 1, or a stereoisomer or a pharmaceutically acceptable salt thereof, to the individual.

29. (canceled)

30. The method of claim 28, wherein the PU.1-mediated disease is leukemia or fibrosis.

31. (canceled)
